(19) United States
(12) Patent Application Publication
(10) Pub. No.: US 2007/0270436 A1
(43) Pub. Date: Nov. 22, 2007

(54) NOVEL AMINO- AND IMINO-ALKYLPIPERAZINES

(75) Inventors: Amedeo Leonardi, Milan (IT); Gianni Motta, Barlassina (IT); Carlo Riva, Varese (IT); Luciano Guarneri, Garbagnate Milanese (IT)

Correspondence Address: DARBY & DARBY P.C., P.O. BOX 770, Church Street Station, New York, NY, 10008-0770, US

(73) Assignee: Recordati Ireland Limited, Ringaskiddy (IE)

(21) Appl. No.: 11/751,322

(22) Filed: May 21, 2007

Related U.S. Application Data
(60) Provisional application No. 60/802,738, filed on May 22, 2006.

(51) Int. Cl. A61K 31/496; C07D 403/02; C07D 405/02

(52) U.S. Cl. 514/254.05; 514/254.09; 514/254.11; 544/364; 544/373

(57) ABSTRACT

Described are novel amino- and iminoalkyl piperazines having affinity for serotonergic receptors and pharmacological compositions thereof. These compounds and their isomers, including optical isomers, enantiomers, diastereomers, N-oxides, polymorphs, hydrates, solvates and pharmaceutically acceptable salts are useful in the treatment of patients with neuromuscular dysfunction of the lower urinary tract and CNS diseases and/or disorders associated with 5-HT_{1A} receptor dysfunction.

### CROSS REFERENCE TO PRIOR APPLICATION

This application claims priority to U.S. Provisional Application No. 60/802,738 filed on May 22, 2006, which is incorporated by reference herein in its entirety.

### FIELD OF THE INVENTION

This invention relates to novel 1,4-disubstituted piperazines having affinity for serotonergic receptors, to pharmaceutical compositions thereof and to uses for such compounds and compositions in the treatment of urinary tract disorders and CNS disorders associated with serotonergic dysfunction.

### BACKGROUND OF THE INVENTION

In mammals, micturition (urination) is a complex process that requires the integrated action of the bladder, its internal and external sphincters, the musculature of the pelvic floor and neurological control over these muscles at three levels 1) in the bladder wall or sphincter itself, 2) in the autonomic centres of the spinal cord, and 3) in the central nervous system at the level of the pontine micturition centre (PMC) in the brainstem (pons) under the control of the cerebral cortex (De Groat, Neurobiology of Incontinence, Ciba Foundation Symposium 151:27, 1990). Micturition results from contraction of the detrusor muscle, which consists of interlacing smooth-muscle fibres, under the control of the parasympathetic autonomic system originating from the sacral spinal cord. A simple voiding reflex is triggered by sensory nerves for pain, temperature and distension that run from the bladder to the sacral spinal cord. However, sensory tracts from the bladder reach the PMC too, generating nerve impulses that normally suppress the sacral spinal suppression of cortical inhibition of the reflex arc, and relaxing the muscles of the pelvic floor and external sphincter. Finally, the detrusor muscle contracts and voiding occurs. Abnormalities of lower-urinary tract function, e.g., dysuria, incontinence and enuresis, are common in the general population. Dysuria includes urinary frequency, nocturia and urgency, and may be caused by cystitis (including interstitial cystitis), prostatitis or benign prostatic hyperplasia (BPH) (which affects about 70% of elderly males), or by neurological disorders. Incontinence syndromes include stress incontinence, urgency incontinence, overflow incontinence and mixed incontinence. Enuresis refers to the involuntary passage of urine at night or during sleep.

Previously, treatment of neuromuscular dysfunction of the lower urinary tract, also defined as overactive bladder, involved administration of compounds that act directly on the bladder muscles, such as flavoxate, a spasmolytic drug (Ruffman, J. Int. Med. Res. 16:317, 1988) which is also active on the PMC (Guarneri et al., Drugs of Today, 30:91, 1994), or anticholinergic (antimuscarinic) compounds such as oxybutynin (Andersson, Drugs 36:477, 1988) and tolterodine (Nilvebrant, Life Sci. 68(22-23): 2549, 2001). The use of α1-adrenergic receptor antagonists for the treatment of BPH is common too, but is based on a different mechanism of action (Lepor, Urology, 42:483, 1993). However, treatments that involve direct inhibition of the pelvic musculature (including the detrusor muscle) may have unwanted side effects, such as incomplete voiding or accommodation paralysis, tachycardia and dry mouth (Andersson, Drugs 35:477, 1988). Thus, it would be preferable to utilize compounds that act via the central nervous system to, for example, affect the sacral spinal reflex and/or the PMC inhibition pathways in a manner that restores normal functioning of the micturition mechanism.

U.S. Pat. No. 5,346,896 discloses 5-HT_{1A} binding agents which may be used in the treatment of CNS disorders, such as, for example, anxiety.

U.S. Pat. Nos. 6,239,135 and 5,358,958 and U.S. Patent Application Publication No. 2001/0003749 A1 disclose aryl piperazine compounds that bind to 5-HT_{1A} receptors.

### SUMMARY OF THE INVENTION

The present invention provides compounds of formula I: 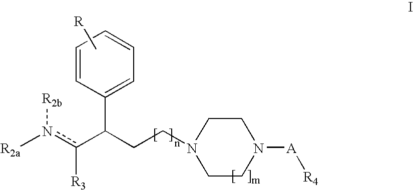
wherein

R is hydrogen or one or more substituents independently selected from the group consisting of alkyl, alkoxy, alkylthio, hydroxy, halo, polyhaloalkoxy, nitro, amino, cyano, aminocarbonyl, and alkylcarbonyl;

R₂ₐ is selected from the group consisting of hydrogen, alkyl, alkenyl, alkynyl, cycloalkyl, heteroarylalkyl, arylalkyl, hydroxy, alkoxy, alkanoyloxy, arylalkoxy, heteroarylalkoxy, alkoxycarbonyl, alkoxythiocarbonyl arylthiocarbonyl, heteroarylthiocarbonyl, alkanoyl, formyl, alkylthiocarbonyl, arylcarbonyl, alkoxyalkyl, aminocarbonyl, carbamoyl, aminothiocarbonyl, N-alkylaminocarbonyl, N-alkylaminothiocarbonyl, N,N-dialkylaminocarbonyl, N,N-dialkylaminothiocarbonyl, and heterocyclylcarbonyl or R₂ₐ is heteroarylalkyl or arylalkyl optionally substituted with one or more substituents R as given above;

R_{2b} is not present, or is selected from the group consisting of hydrogen, alkyl, alkenyl, alkynyl, cycloalkyl, heteroarylalkyl, arylalkyl, alkanoyl, formyl, alkylthiocarbonyl, arylcarbonyl, or R_{2b} is heteroarylalkyl or arylalkyl optionally substituted with one or more substituents R as given above;

R₃ is alkyl, alkenyl, alkynyl or cycloalkyl;

or

R₂ₐ and R₃ together with the nitrogen to which R₂ is attached and the carbon to which R₃ is attached, are linked to form a five membered saturated or unsaturated heterocyclic ring optionally substituted with one or more alkyl groups;

R₄ is aryl or heterocyclyl, each being optionally substituted with one or more substituent R as defined above;

A is a bond or (CH₂)ₙ;

m and n=1 or 2;

[symbol] is a single bond when R_{2b} is present or a double bond when R_{2b} is not present; or

an enantiomer, optical isomer, diastereomer, N-oxide (e.g., N-piperazine oxide), crystalline form, hydrate, solvate or pharmaceutically acceptable salt thereof.

The invention also includes metabolites of the foregoing compounds of formula I having the same type of activity, hereinafter referred to as active metabolites.

The present invention also contemplates prodrugs which are metabolized in the body to generate any of the foregoing compounds.

In another embodiment, the present invention provides pharmaceutical compositions comprising compounds of formula I, enantiomers, diastereomers, N-piperazine oxides, crystalline forms, hydrates, solvates or pharmaceutically acceptable salts of such compounds of formula I, in admixture with one or more pharmaceutically acceptable diluent or carrier.

Another embodiment of the invention is a method for reducing the frequency of bladder contractions due to bladder distension in a mammal (such as a human) in need thereof by administering an effective amount of at least one compound of the present invention.

Another embodiment of the invention is a method for increasing urinary bladder capacity in a mammal (such as a human) in need thereof by administering an effective amount of at least one compound of the present invention.

Another embodiment of the invention is a method for treating disorders or conditions of the urinary tract in a mammal (such as a human) in need thereof by administering an effective amount of at least one compound of the present invention to ameliorate at least one condition among urinary urgency, overactive bladder, increased urinary frequency, decreased urinary compliance (decreased bladder storage capacity), cystitis (including interstitial cystitis), incontinence, urine leakage, enuresis, dysuria, urinary hesitancy and difficulty in emptying the bladder.

When used in methods of treating the aforementioned disorders, compounds of the invention may be administered in combination with other drugs known to be useful in the treatment of urinary tract disorders or for the therapy of lower urinary tract symptoms, which may or may not be associated with BPH, such as, for example, antimuscarinics and α₁-adrenergic antagonists. Compounds of the invention may be administered, for example, with the antimuscarinic drugs oxybutynin, tolterodine, darifenacin, solifenacin, imidafenacin, fesoterodine, propiverine, trospium, zamifenacin, vamicamide and temiverine. Analogously, the compounds of the invention may be administered with α₁-adrenergic antagonists such as, for example, prazosin, doxazosin, terazosin, alfuzosin, tamsulosin and silodosin. Additional α₁ antagonists suitable for administration in combination with the compounds of the present invention are found, for example, in U.S. Pat. Nos. 5,798,362; 6,306,861; 6,365,591 and 6,403,594, which are incorporated by reference herein in their entirety.

In yet another embodiment, the present invention provides a method for treating a central nervous system (CNS) disorder due to serotonergic dysfunction in a mammal (such a human) in need thereof by administering an effective amount of at least one compound of the present invention. Such CNS disorders include, but are not limited to, anxiety, depression, hypertension, sleep/wake cycle disorders, feeding, behaviour, sexual dysfunction, and cognition disorders associated with or originating from stroke, injury, dementia, neurological development, Alzheimer disease (AD), attention-deficit hyperactivity disorders (ADHD), drug addiction, drug withdrawal, irritable-bowel syndrome, and symptoms caused by withdrawal or partial withdrawal from the use of nicotine or tobacco. Treatment may be effected by delivering a compound of the invention to the environment of a 5-HT_{1A} serotonergic receptor, for example, to the extracellular medium (or by systemically or locally administering to a mammal possessing such a 5-HT_{1A} receptor) an amount of a compound of the invention effective in the treatment of the aforementioned disorders.

In a preferred embodiment, the invention provides methods for treating a mammal (including a human) suffering from a urinary tract disorder by administering at least one compound of the invention to the environment of a 5-HT_{1A} receptor in an amount effective to increase the duration of bladder quiescence. More preferred are treatment methods wherein the increase in the duration of bladder quiescence is accomplished with little or no effect on micturition pressure (e.g., little or no decrease or increase in micturition pressure).

### BRIEF DESCRIPTION OF THE DRAWINGS

FIG. 1 shows the results for cystometry studies in conscious rats after administration of the compound of Example 16.

FIG. 2 shows the results for cystometry studies in conscious rats after administration of the compound of Example 24.

FIG. 3 shows the results for cystometry studies in conscious rats after administration of oxybutynin.

### DETAILED DESCRIPTION OF THE INVENTION

### Compounds of Formula I

The present invention is related to compounds of Formula I as disclosed above. Unless explicitly stated or apparent from context, the phrase “a compound(s) of Formula I” includes the enantiomers, diastereomers, N-oxides, crystalline forms, hydrates, solvates or pharmaceutically acceptable salts of Formula I, as well as active metabolites of these compounds having the same type of activity.

The term “alkyl” refers, either alone or within other terms such as “arylalkyl” to linear or branched radicals having from one to about twenty carbon atoms or, preferably, from one to about twelve carbon atoms. More preferred alkyl radicals are “lower alkyl” radicals having from one to about ten carbon atoms. Most preferred are lower alkyl radicals having from one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkenyl” refers to linear or branched radicals having at least one carbon-carbon double bond of from two to about twenty carbon atoms or, preferably, from two to about twelve carbon atoms. More preferred alkenyl radicals are “lower alkenyl” radicals having from two to about six carbon atoms. Examples of such radicals include ethenyl, propenyl, butenyl, and the like.

The term “alkynyl” refers to linear or branched radicals having at least one carbon-carbon triple bond of from two to about twenty carbon atoms or, preferably, from two to about twelve carbon atoms. More preferred alkynyl radicals are “lower alkynyl” radicals having from two to about six carbon atoms. Examples of such radicals include, ethynyl, propynyl, butynyl, and the like.

The term “halo” refers to halogens such as fluorine, chlorine, bromine or iodine atoms.

The term “alkoxy” refers to linear or branched oxy-containing radicals each having alkyl portions of from one to about ten carbon atoms, such as methoxy radical. Preferred alkoxy radicals are “lower alkoxy” radicals having from one to six carbon atoms. Examples of such radicals include methoxy, ethoxy, propoxy, butoxy and tert-butoxy.

The term “polyhaloalkoxy” refers to “alkoxy” radicals further substituted with more than one halo atoms, such as fluoro, chloro or bromo. Examples of such radicals include trifluoromethoxy, trifluoroethoxy.

The term “aryl,” alone or in combination, refers to a carbocyclic aromatic system containing one, two or three rings wherein such rings may be attached together in a pendant manner or may be fused. The term “aryl” includes aromatic radicals such as phenyl, naphthyl, tetrahydronaphthyl, indane and biphenyl.

The terms “heterocyclic” and “heterocyclo” refer to saturated, partially saturated and unsaturated heteroatom-containing ring-shaped radicals, where the heteroatoms may be selected from nitrogen, sulfur and oxygen. Examples of saturated heterocyclic radicals include saturated heteromonocylic groups containing 1 to 4 nitrogen atoms (e.g., pyrrolidinyl, imidazolidinyl, piperidinyl, piperazinyl); saturated heteromonocyclic groups containing 1 to 2 oxygen atoms and 1 to 3 nitrogen atoms (e.g., morpholinyl); saturated heteromonocyclic groups containing 1 to 2 sulfur atoms and 1 to 3 nitrogen atoms (e.g., thiazolidinyl). Examples of partially saturated heterocyclic radicals include dihydrothiophene, dihydropyran, dihydrofuran and dihydrothiazole. As used herein, the terms “heterocycle” and “heterocyclic” encompass the term “heteroaryl.”

“Heteroaryl” refers to unsaturated heterocyclic radicals. Examples of “heteroaryl” radicals include unsaturated 5 to 6 membered heteromonocyclic groups containing 1 to 4 nitrogen atoms, for example, pyrrolyl, pyrrolinyl, imidazolyl, pyrazolyl, 2-pyridyl, 3-pyridyl, 4-pyridyl, pyrimidyl, pyrazinyl, pyridazinyl, triazolyl (e.g., 4H-1,2,4-triazolyl, 1H-1,2,3-triazolyl, 2H-1,2,3-triazolyl)tetrazolyl (e.g., 1H-tetrazolyl, 2H-tetrazolyl); unsaturated condensed heterocyclic groups containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl); unsaturated 3 to 6-membered heteromonocyclic groups containing an oxygen atom, for example, pyranyl, 2-furyl, 3-furyl; unsaturated 5 to 6-membered heteromonocyclic groups containing a sulfur atom, for example, 2-thienyl, 3-thienyl; unsaturated 5- to 6-membered heteromonocyclic groups containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl); unsaturated condensed heterocyclic groups containing 1 to 2 oxygen atoms and 1 to 3 nitrogen atoms (e.g., benzoxazolyl, benzoxadiazolyl); unsaturated 5 to 6-membered heteromonocyclic groups containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl); unsaturated condensed heterocyclic groups containing 1 to 2 sulfur atoms and 1 to 3 nitrogen atoms (e.g., benzothiazolyl, benzothiadiazolyl) and the like. The term “heteroaryl” also refers to radicals where heterocyclic radicals are fused with aryl radicals. Examples of such fused bicyclic radicals include benzofuran, benzothiophene, and the like. Said “heterocyclic group” may have 1 to 3 substituents such as, for example and without limitation, lower alkyl, hydroxy, oxo, amino and lower alkylamino. Preferred heterocyclic radicals include five to ten membered fused or unfused radicals. Examples of heteroaryl radicals include benzofuryl, 2,3-dihydrobenzofuryl, benzothienyl, indolyl, dihydroindolyl, benzimidazolyl chromanyl, benzopyran, thiochromanyl, benzothiopyran, benzodioxolyl, benzodioxanyl, 2,3-dihydrobenzodioxanyl, pyridyl, thienyl, thiazolyl, oxazolyl, furyl, and pyrazinyl.

The terms “alkanoyl” and “alkylcarbonyl” are synonymous and refer to radicals having a carbonyl radical as defined below, attached to an alkyl radical. Preferred alkanoyl radicals are “lower alkanoyl” radicals having 1-6 carbon atoms. The alkanoyl radicals may be substituted or unsubstituted, such as formyl, acetyl, propionyl(propanoyl), butanoyl(butyryl), isobutanoyl(isobutyryl), valeryl(pentanoyl), isovaleryl, pivaloyl, hexanoyl or the like.

The term “carbonyl”, whether used alone or with other terms, such as “alkylcarbonyl”, denotes —(C═O)—.

The term “alkylcarbonylalkyl”, denotes an alkyl radical substituted with an “alkylcarbonyl” radical.

The term “alkoxycarbonyl” refers to a radical containing an alkoxy radical, as defined above, attached via an oxygen atom to a carbonyl radical, i.e., an ester radical, —C(O)O-Alk. Preferred alkoxycarbonyl radicals are “lower alkoxycarbonyl” radicals having alkoxy radicals of one to six carbon atoms. Examples of such “lower alkoxycarbonyl” ester radicals include substituted or unsubstituted methoxycarbonyl, ethoxycarbonyl, propoxycarbonyl, butoxycarbonyl and hexyloxycarbonyl.

The term “alkanoyloxy” refers to an “alkanoyl” radical as defined above linked to an oxygen radical, to generate an ester group, i.e., —O—C(O)-Alkyl.

The term “aminocarbonyl” when used by itself or with other terms such as “aminocarbonylalkyl,” “N-alkylaminocarbonyl,” “N,N-dialkylaminocarbonyl,” “N-alkyl-N-arylaminocarbonyl,” “N-alkyl-N-hydroxyaminocarbonyl” and “N-alkyl-N-hydroxyaminocarbonylalkyl,” denotes an amide group of the formula —C(═O)N—. The terms “N-alkylaminocarbonyl” and “N,N-dialkylaminocarbonyl” denote aminocarbonyl radicals in which an primary amino group has been substituted with one alkyl radical and two alkyl radicals, respectively. Preferred are “lower alkylaminocarbonyl” having lower alkyl radicals as described above attached to an aminocarbonyl radical.

The term “cycloalkyl” refers to carbocyclic radicals having three to ten carbon atoms. Preferred cycloalkyl radicals are “lower cycloalkyl” radicals having three to seven carbon atoms. Examples include radicals such as cyclopropyl, cyclobutyl, cyclopentyl, cyclohexyl and cycloheptyl. A most preferred cycloalkyl group is cyclohexyl.

The term “alkylthio” refers to radicals containing a linear or branched alkyl radical, of one to ten carbon atoms, attached to a divalent sulfur atom. An example of “alkylthio” is methylthio, (CH₃—S—).

The term “amino” refers to the radical —NH₂.

The terms “N-alkylamino” and “N,N-dialkylamino” denote amino groups which have been substituted with one alkyl radical and with two alkyl radicals, respectively. More preferred alkylamino radicals are “lower alkylamino” radicals having one or two alkyl radicals of one to six carbon atoms, attached to the nitrogen atom. Examples of “alkylamino” include N-methylamino, N-ethylamino, N,N-dimethylamino, N,N-diethylamino or the like.

The term “aralkyl” refers to aryl-substituted alkyl radicals. Preferable aralkyl radicals are “lower aralkyl” radicals having aryl radicals attached to alkyl radicals having one to six carbon atoms. Examples of such radicals include benzyl, diphenylmethyl, triphenylmethyl, phenylethyl and diphenylethyl. The aryl in said aralkyl may be additionally substituted with halo, alkyl, alkoxy, halkoalkyl and haloalkoxy. The terms benzyl and phenylmethyl are interchangeable.

The term “aryloxy” refers to the radical —O-aryl. Examples of such radicals include phenoxy.

The term “aralkoxy” refers to the radical -alkoxy-aryl (i.e., —O-alkyl-aryl). Preferred aralkoxy radicals are “lower aralkoxy” radicals having phenyl radicals attached to a lower alkoxy radical as described above. An example of such radical includes benzyloxy.

The term “cyano” refers to the radical —C≡N.

The term “nitro” refers to the radical —NO₂.

The term “heterocycloalkyl” refers to the radical -Alkyl-Heterocycle.

The term “hydroxy” refers to the radical —OH.

In a preferred embodiment of the invention, variable R of Formula I is a halogen atom. Further preferred is where R is a fluorine atom. Still further preferred is where R is a fluorine atom at the 2-position of the phenyl ring.

Preferably, R₂ₐ is selected from hydrogen, alkyl, aralkyl, hydroxy, alkoxy, alkanoyl, aminocarbonyl, aminothiocarbonyl, N-alkylaminocarbonyl, N-alkylaminothiocarbonyl, N,N-dialkylaminocarbonyl, and N,N-dialkylaminothiocarbonyl. More preferred is where R₂ₐ is selected from hydrogen, alkyl, and alkanoyl. Most preferred is where [symbol] is a single bond and N(R₂ₐ)(R_{2b}) is an alkanoylamino or a N-alkyl-N-alkanoylamino.

Preferred groups for R₃ are substituted or unsubstituted alkyl, alkenyl, alkynyl, or cycloalkyl groups. Further preferred is where R₃ represents a substituted or unsubstituted cyclohexyl group or linear alkyl group. Most preferred is where R₃ represents an unsubstituted cyclohexyl group.

Preferred groups for R₄ are substituted or unsubstituted monocyclic aryl or bicyclic heterocyclic groups.

Also preferred are compounds of Formula I wherein the groups R₁, R₂ₐ, R₃, R₄ and A are simultaneously or independently selected from the following:

R is polyhaloalkoxy, cyano, alkylcarbonyl, or N,N-dialkylaminocarbonyl;

R₂ₐ is alkyl, alkanoyl or alkylthiocarbonyl

R₃ is unsubstituted cycloalkyl, alkyl, or alkenyl; and

R₄ is phenyl substituted with polyhaloalkoxy, alkoxy or halogen, or is benzodioxanyl; and

A is a bond or methylene unit.

Also preferred are the following Formula I compounds:
1-Cyclohexyl-2-(2-fluorophenyl)-4-[4-(2-methoxyphenyl)piperazin-1-yl]butan-1-one oxime;
1-Cyclohexyl-2-(2-fluorophenyl)-4-[4-(2-methoxyphenyl)piperazin-1-yl]butan-1-one;
1-Cyclohexyl-2-(2-fluorophenyl)-4-[4-(2-methoxyphenyl)piperazin-1-yl]butan-1-one;
N-{1-Cyclohexyl-2-(2-fluorophenyl)-4-[4-(2-methoxyphenyl)piperazin-1-yl]butyl}-O-methylhydroxylamine;
(RS,SR)-N-{1-Cyclohexyl-2-(2-fluorophenyl)-4-[4-(2-methoxyphenyl)piperazin-1-yl]butyl}-O-methylhydroxylamine;
(RS,RS)-N-{1-Cyclohexyl-2-(2-fluorophenyl)-4-[4-(2-methoxyphenyl)piperazin-1-yl]butyl}-O-methylhydroxylamine;
(RS,SR)-1-Cyclohexyl-2-(2-fluorophenyl)-4-[4-(2-methoxyphenyl)piperazin-1-yl]butylamine;
(RS,RS)-1-Cyclohexyl-2-(2-fluorophenyl)-4-[4-(2-methoxyphenyl)piperazin-1-yl]butylamine;
(1R,2S) 1-Cyclohexyl-2-(2-fluorophenyl)-4-[4-(2-methoxyphenyl)piperazin-1-yl]butylamine;
(1S,2R)-1-Cyclohexyl-2-(2-fluorophenyl)-4-[4-(2-methoxyphenyl)piperazin-1-yl]butylamine;
N-{1-Cyclohexyl-2-(2-fluorophenyl)-4-[4-(2-methoxyphenyl)piperazin-1-yl]butyl}formamide;
N-{(RS,SR)-1-Cyclohexyl-2-(2-fluorophenyl)-4-[4-(2-methoxyphenyl)piperazin-1-yl]butyl}-acetamide;
N-Acetyl-N-{(RS,SR)-1-cyclohexyl-2-(2-fluorophenyl)-4-[4-(2-methoxyphenyl)piperazin-1-yl]butyl}acetamide;
N-{(RS,RS)-1-Cyclohexyl-2-(2-fluorophenyl)-4-[4-(2-methoxyphenyl)piperazin-1-yl]butyl}-acetamide;
(RS,SR)-{1-Cyclohexyl-2-(2-fluorophenyl)-4-[4-(2-methoxyphenyl)piperazin-1-yl]butyl}methylamine;
(RS,RS)-{1-Cyclohexyl-2-(2-fluoro-phenyl)-4-[4-(2-methoxyphenyl)piperazin-1-yl]butyl}methylamine;
{(RS,SR)-1-Cyclohexyl-2-(2-fluorophenyl)-4-[4-(2-methoxyphenyl)piperazin-1-yl]butyl}dimethylamine;
(RS,SR)-{1-Cyclohexyl-2-(2-fluorophenyl)-4-[4-(2-methoxyphenyl)piperazin-1-yl]butyl}iso-propylamine;
(RS,SR)-N-Benzyl-{1-cyclohexyl-2-(2-fluorophenyl)-4-[4-(2-methoxyphenyl)piperazin-1-yl]butyl}amine;
(RS,RS)-N-Benzyl-{1-cyclohexyl-2-(2-fluorophenyl)-4-[4-(2-methoxyphenyl)piperazin-1-yl]butyl}amine;
{(RS,SR)-1-Cyclohexyl-2-(2-fluorophenyl)-4-[4-(2-methoxyphenyl)piperazin-1-yl]butyl}furan-2-ylmethylamine;
(RS,SR)-1-Cyclohexyl-2-(2-fluorophenyl)-4-[4-(2-methoxyphenyl)piperazin-1-yl]butyl}-bis-furan-2-ylmethylamine;
(RS,SR)-N-{-1-Cyclohexyl-2-(2-fluorophenyl)-4-[4-(2-methoxyphenyl)piperazin-1-yl]butyl}-N-methylacetamide;
(RS,RS)-N-{-1-Cyclohexyl-2-(2-fluorophenyl)-4-[4-(2-methoxyphenyl)piperazin-1-yl]butyl}-N-methylacetamide;
(RS,SR)-{1-Cyclohexyl-2-(2-fluorophenyl)-4-[4-(2-methoxyphenyl)piperazin-1-yl]butyl}carbamic acid methyl ester;
{(RS,SR)-1-Cyclohexyl-2-(2-fluorophenyl)-4-[4-(2-methoxyphenyl)piperazin-1-yl]butyl}urea;
1-{(RS,SR)-1-Cyclohexyl-2-(2-fluorophenyl)-4-[4-(2-methoxyphenyl)piperazin-1-yl]butyl}-3-methylurea;
{(RS,SR)-N-1-cyclohexyl-2-(2-fluorophenyl)-4-[4-(2-methoxyphenyl)piperazin-1-yl]butyl}morpholine-4-carboxamide;
3-{(RS,SR)-1-Cyclohexyl-2-(2-fluorophenyl)-4-[4-(2-methoxy-phenyl)piperazin-1-yl]butyl}-1,1-dimethylurea;
1-{(RS,SR)-1-Cyclohexyl-2-(2-fluorophenyl)-4-[4-(2-methoxyphenyl)piperazin-1-yl]butyl}-1,3,3-trimethylurea;
1-{(RS,SR)-1-Cyclohexyl-2-(2-fluorophenyl)-4-[4-(2-methoxyphenyl)piperazin-1-yl]butyl}-3-methylthiourea;
3-{(RS,SR)-1-Cyclohexyl-2-(2-fluorophenyl)-4-[4-(2-methoxyphenyl)piperazin-1-yl]butyl}-1,1-dimethylthiourea;
3-{(1R,2S) or (1S,2R)-1-Cyclohexyl-2-(2-fluorophenyl)-4-[4-(2-methoxyphenyl)piperazin-1-yl]butyl}-1,1-dimethylthiourea;
3-{(1S,2R) or (1R,2S)-1-Cyclohexyl-2-(2-fluorophenyl)-4-[4-(2-methoxyphenyl)piperazin-1-yl]butyl}-1,1-dimethylthiourea;
2-{1-Cyclohexyl-[(E)-hydroxyiminomethyl]-3-[4-(2,3-dihydrobenzo[1,4]dioxin-5-yl)piperazin-1-yl]propyl}benzonitrile;
(R,S)-2-{1-{Cyclohexyl-[(E)-methoxyimino]methyl}-3-[4-(2,3-dihydrobenzo[1,4]dioxin-5-yl)-piperazin-1-yl]propyl}benzonitrile;
E(Z)-5-[4-(2,3-Dihydrobenzo[1,4]dioxin-5-yl)piperazin-1-yl]-3-(2-fluorophenyl)pentan-2-one oxime;
5-[4-(2,3-Dihydrobenzo[1,4]dioxin-5-yl)piperazin-1-yl]-3-(2-fluorophenyl)pentan-2-one O-methyloxime;
(RS,SR)-[4-(2,3-Dihydrobenzo[1,4]dioxin-5-yl)piperazin-1-yl]-2-(2-fluorophenyl)-1-methylbutylamine;
(RS,RS)-[4-(2,3-Dihydrobenzo[1,4]dioxin-5-yl)piperazin-1-yl]-2-(2-fluorophenyl)-1-methylbutylamine;
(RS,SR)-[4-[4-(2,3-Dihydrobenzo[1,4]dioxin-5-yl)piperazin-1-yl]-2-(2-fluorophenyl)-1-methylbutyl]methylamine;
(RS,RS)-[4-[4-(2,3-Dihydrobenzo[1,4]dioxin-5-yl)piperazin-1-yl]-2-(2-fluorophenyl)-1-methylbutyl]methylamine;
(E)-3-(2-Fluorophenyl)-5-{4-[2-(2,2,2-trifluoroethoxy)phenyl]piperazin-1-yl}pentan-2-one oxime;
(Z)-3-(2-Fluorophenyl)-5-{4-[2-(2,2,2-trifluoroethoxy)phenyl]piperazin-1-yl}pentan-2-one oxime;
(E)-3-(2-Fluorophenyl)-5-{4-[2-(2,2,2-trifluoroethoxy)phenyl]piperazin-1-yl}pentan-2-one O-methyloxime;
(Z)-3-(2-Fluorophenyl)-5-{4-[2-(2,2,2-trifluoroethoxy)phenyl]piperazin-1-yl}pentan-2-one O-methyloxime;
(RS,SR)-2-(2-Fluorophenyl)-1-methyl-4-{4-[2-(2,2,2-trifluoroethoxy)phenyl]piperazin-1-yl}butylamine;
(RS,RS)-2-(2-Fluorophenyl)-1-methyl-4-{4-[2-(2,2,2-trifluoroethoxy)phenyl]piperazin-1-yl}butylamine;
5-{3-[4-(2,3-Dihydrobenzo[1,4]dioxin-5-yl)piperazin-1-yl]-1-phenylpropyl}pyrrolidin-2-one;
(RS,SR)-5-{3-[4-(2,3-Dihydrobenzo[1,4]dioxin-5-yl)piperazin-1-yl]-1-phenylpropyl}-1-ethylpyrrolidin-2-one;
(RS,RS)-5-{3-[4-(2,3-Dihydrobenzo[1,4]dioxin-5-yl)piperazin-1-yl]-1-phenylpropyl}-1-ethylpyrrolidin-2-one;
5-{3-[4-(2,3-Dihydrobenzo[1,4]dioxin-5-yl)piperazin-1-yl]-1-phenylpropyl}-1-ethylpyrrolidin-2-one;
1-(2,3-Dihydrobenzo[1,4]dioxin-5-yl)-4-[3-(5-methylisoxazol-3-yl)-3-phenylpropyl]piperazine;
N-[1-Cyclohexyl-4-[4-(2,3-dihydrobenzo[1,4]dioxin-5-yl)piperazin-1-yl]-2-(2-fluorophenyl)butyl]formamide;
(RS,SR)-1-Cyclohexyl-4-[4-(2,3-dihydrobenzo[1,4]dioxin-5-yl)piperazin-1-yl]-2-(2-fluorophenyl)butylamine;
1-Cyclohexyl-4-[4-(2,3-dihydrobenzo[1,4]dioxin-5-yl)piperazin-1-yl]-2-(2-fluoro-phenyl)butylmethylamine;
N-[1-Cyclohexyl-4-[4-(2,3-dihydrobenzo[1,4]dioxin-5-yl)piperazin-1-yl]-2-(2-fluoro-phenyl)butyl]acetamide;
N-[1-Cyclohexyl-4-[4-(2,3-dihydrobenzo[1,4]dioxin-5-yl)piperazin-1-yl]-2-(2-fluoro-phenyl)butyl]-N-methylacetamide;
N-[4-[4-(2-Bromo-5-methoxybenzyl)piperazin-1-yl]-1-cyclohexyl-2-(2-fluorophenyl)butyl]formamide;
4-[4-(2-Bromo-5-methoxybenzyl)piperazin-1-yl]-1-cyclohexyl-2-(2-fluorophenyl)butylamine;
4-[4-(2-Bromo-5-methoxybenzyl)piperazin-1-yl]-1-cyclohexyl-2-(2-fluorophenyl)butylmethylamine;
N-[4-[4-(2-Bromo-5-methoxybenzyl)piperazin-1-yl]-1-cyclohexyl-2-(2-fluoro-phenyl)butyl]acetamide;
N-[4-[4-(2-Bromo-5-methoxybenzyl)piperazin-1-yl]-1-cyclohexyl-2-(2-fluorophenyl)butyl]-N-methylacetamide;
N-[4-[4-(1H-Benzimidazol-4-yl)piperazin-1-yl]-1-cyclohexyl-2-(2-fluoro-phenyl)butyl]formamide;
4-[4-(1H-Benzimidazol-4-yl)piperazin-1-yl]-1-cyclohexyl-2-(2-fluorophenyl)butylamine;
4-[4-(1H-Benzimidazol-4-yl)piperazin-1-yl]-1-cyclohexyl-2-(2-fluorophenyl)butylmethylamine;
N-[4-[4-(1H-Benzimidazol-4-yl)piperazin-1-yl]-1-cyclohexyl-2-(2-fluorophenyl)butyl]acetamide;
N-[4-[4-(1H-Benzimidazol-4-yl)piperazin-1-yl]-1-cyclohexyl-2-(2-fluorophenyl)butyl]-N-methylacetamide;
N-{1-Cyclohexyl-2-(2-fluorophenyl)-4-[4-(1H-indol-4-yl)piperazin-1-yl]butyl}formamide;
1-{(RS,SR)-1-Cyclohexyl-2-(2-fluorophenyl)-4-[4-(2-methoxyphenyl)piperazin-1-yl]butyl}-1,3,3-trimethylthiourea, or
   enantiomer, optical isomer, diastereomer, N-oxide, crystalline form, hydrate, solvate or pharmaceutically acceptable salt thereof.

### Salts, Solvates, Stereoisomers, Derivatives, Prodrugs and Active Metabolites of the Novel Compounds of the Invention

The present invention encompasses salts, solvates, stereoisomers, prodrugs and active metabolites of the compounds of formula I.

The term “salts” can include acid addition salts or addition salts of free bases. Preferably, the salts are pharmaceutically acceptable. Examples of acids which may be employed to form pharmaceutically acceptable acid addition salts include, but are not limited to, salts derived from inorganic acids such as nitric, phosphoric, sulfuric, or hydrobromic, hydroiodic, hydrofluoric, phosphorous, as well as salts derived from organic acids such as aliphatic mono- and dicarboxylic acids, phenyl-substituted alkanoic acids, hydroxyl alkanoic acids, alkanedioic acids, aromatic acids, aliphatic and aromatic sulfonic acids, and acetic, maleic, succinic, or citric acids. Non-limiting examples of such salts include napadisylate, besylate, sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge, et al. “Pharmaceutical Salts,” J. Pharma. Sci. 1977; 66:1).

The phrase “pharmaceutically acceptable,” as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the federal or a state government or listed in the U.S. Pharmacopoeia or other generally recognized pharmacopeias for use in mammals, and more particularly in humans.

Pharmaceutically acceptable salts of a compound of formula I may be readily prepared by using a desired acid or base, as appropriate. The salt may precipitate from solution and be collected by filtration or may be recovered by evaporation of the solvent. For example, an aqueous solution of an acid such as hydrochloric acid may be added to an aqueous suspension of a compound of formula I and the resulting mixture evaporated to dryness (lyophilized) to obtain the acid addition salt as a solid. Alternatively, a compound of formula I may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Acid addition salts of the compounds of formula I may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid.

Compounds of the invention may have both a basic and an acidic center and therefore be in the form of zwitterions.

Those skilled in the art of organic chemistry will appreciate that many organic compounds can form complexes with solvents in which they are reacted or from which they are precipitated or crystallized. These complexes are known as “solvates.” For example, a complex with water is known as a “hydrate.” Solvates of the compound of the invention are within the scope of the invention. The salts of the compound of formula I may form solvates (e.g., hydrates) and the invention also includes all such solvates. The meaning of the word “solvates” is well known to those skilled in the art as a compound formed by interaction of a solvent and a solute (i.e., salvation). Techniques for the preparation of solvates are well established in the art (see, for example, Brittain. Polymorphism in Pharmaceutical solids. Marcel Decker, New York, 1999).

Certain compounds of formula I can exist in as many as four stereoisomers, which may be present in racemic mixtures or in any other combination. Racemic mixtures can be subjected to methods for enantiomeric enrichment, to yield compositions enriched in a particular enantiomer, or resolved to a composition comprising a single enantiomer. Enantiomeric enrichment can be expressed as percent ee (enantiomeric excess) as defined below.

As used herein, the term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. As used herein, the term “optical isomer” is equivalent to the term “enantiomer.” Compounds that are stereoisomers of one another, but are not enantiomers of one another, are called diastereomers. The terms “racemate” or “racemic mixture” refer to a mixture of equal parts of enantiomers. The term “chiral center” refers to a carbon atom to which four different groups are attached. The term “enantiomeric enrichment” as used herein refers to the increase in the amount of one enantiomer as compared to the other. A convenient method of expressing the enantiomeric enrichment achieved is the concept of enantiomeric excess, or “ee,” which is found using the following equation:

$$ee = \frac{E 1 - E 2}{E 1 + E 2} * 100$$

wherein E1 is the amount of the first enantiomer and E2 is the amount of the second enantiomer. Thus, if the initial ratio of the two enantiomers is 50:50, such as is present in a racemic mixture, and an enantiomeric enrichment sufficient to produce a final ratio of 50:30 is achieved, the ee with respect to the first enantiomer is 25%. However, if the final ratio is 90:10, the ee with respect to the first enantiomer is 80%. According to one embodiment of the invention, an ee of greater than 90% is preferred, an ee of greater than 95% is most preferred and an ee of greater than 99% is most especially preferred. Enantiomeric enrichment is readily determined by one of ordinary skill in the art using standard techniques and procedures, such as high performance liquid chromatography with a chiral column. Choice of the appropriate chiral column, eluent and conditions necessary to effect separation of the enantiomeric pair is within the knowledge of one of ordinary skill in the art. In addition, the enantiomers of compounds of formula I can be resolved by one of ordinary skill in the art using standard techniques well known in the art, such as those described by J. Jacques, et al., “Enantiomers, Racemates, and Resolutions,” John Wiley and Sons, Inc., 1981. Examples of resolutions include recrystallization techniques or chiral chromatography.

Diastereisomers differ in both physical properties and chemical reactivity. A mixture of diastereomers can be separated into enantiomeric pairs based on solubility, fractional crystallization or chromatographic properties, e.g., thin layer chromatography, column chromatography or HPLC.

Purification of complex mixtures of diastereomers into enantiomers typically requires two steps. In a first step, the mixture of diastereomers is resolved into enantiomeric pairs, as described above. In a second step, enantiomeric pairs are further purified into compositions enriched for one or the other enantiomer or, more preferably resolved into compositions comprising pure enantiomers. Resolution of enantiomers typically requires reaction or molecular interaction with a chiral agent, e.g., solvent or column matrix. Resolution may be achieved, for example, by converting the mixture of enantiomers, e.g., a racemic mixture, into a mixture of diastereomers by reaction with a pure enantiomer of a second agent, i.e., a resolving agent. The two resulting diasteromeric products can then be separated. The separated diastereomers are then reconverted to the pure enantiomers by reversing the initial chemical transformation.

Resolution of enantiomers can also be accomplished by differences in their non-covalent binding to a chiral substance, e.g., by chromatography on homochiral adsorbants. The noncovalent binding between enantiomers and the chromatographic adsorbant establishes diastereomeric complexes, leading to differential partitioning in the mobile and bound states in the chromatographic system. The two enantiomers therefore move through the chromatographic system, e.g, column, at different rates, allowing for their separation.

Chiral resolving columns are well known in the art and are commercially available (e.g., from MetaChem Technologies Inc., a division of ANSYS Technologies, Inc., Lake Forest, Calif.). Enantiomers can be analyzed and purified using, for example, chiral stationary phases (CSPs) for HPLC. Chiral HPLC columns typically contain one form of an enantiomeric compound immobilized to the surface of a silica packing material.

D-phenylglycine and L-leucine are examples of Type I CSPs and use combinations of pi-pi interactions, hydrogen bonds, dipole-dipole interactions, and steric interactions to achieve chiral recognition. To be resolved on a Type I column, analyte enantiomers must contain functionality complementary to that of the CSP so that the analyte undergoes essential interactions with the CSP. The sample should preferably contain one of the following functional groups: π-acid or π-base, hydrogen bond donor and/or acceptor, or an amide dipole. Derivatization is sometimes used to add the interactive sites to those compounds lacking them. The most common derivatives involve the formation of amides from amines and carboxylic acids.

The MetaChiral ODM™ is an example of a type II CSP. The primary mechanisms for the formation of solute-CSP complexes is through attractive interactions, but inclusion complexes also play an important role. Hydrogen bonding, pi-pi interactions, and dipole stacking are important for chiral resolution on the MetaChiral™ ODM. Derivatization maybe necessary when the solute molecule does not contain the groups required for solute-column interactions. Derivatization, usually to benzylamides, may be required for some strongly polar molecules like amines and carboxylic acids, which would otherwise interact strongly with the stationary phase through non-specific-stereo interactions.

Compounds of formula I can be separated into diastereomeric pairs by, for example, separation by column chromatography or TLC on silica gel. These diastereomeric pairs are referred to herein as diastereomer with upper TLC Rf; and diastereomer with lower TLC Rf. The diastereomers can further be enriched for a particular enantiomer or resolved into a single enantiomer using methods well known in the art, such as those described herein.

The relative configuration of the diastereomeric pairs can be deduced by the application of theoretical models or rules (e.g., Cram's rule, the Felkin-Ahn model) or using more reliable three-dimensional models generated by computational chemistry programs. In many instances, these methods are able to predict which diasteromer is the energetically favored product of a chemical transformation. As an alternative, the relative configuration of the diastereomeric pairs can be indirectly determined by discovering the absolute configurations of a single enantiomer in one (or both) of the diastereomeric pair(s).

The absolute configuration of the stereocenters can be determined by very well known method to those skilled in the art (e.g., X-Ray diffraction, circular dichroism). Determination of the absolute configuration can be useful also to confirm the predictability of theoretical models and can be helpful to extend the use of these models to similar molecules prepared by reactions with analogous mechanisms (e.g., ketone reductions and reductive amination of ketones by hydrides).

The present invention also encompasses stereoisomers of the syn-anti type, and mixtures thereof encountered when an oxime or similar group is present. The group of highest Cahn-Ingold-Prelog priority attached to one of the terminal doubly bonded atoms of the oxime, is compared with hydroxyl group of the oxime. The stereoisomer is designated as Z (zusammen=together) or Syn if the oxime hydroxyl lies on the same side of a reference plane passing through the C═N double bond as the group of highest priority; the other stereoisomer is designated as E (entgegen=opposite) or Anti.

It will be appreciated by those skilled in the art that it may be desirable to use protected derivatives of intermediates used in the preparation of the compounds of formula I. Protection and deprotection of functional groups may be performed by methods known in the art (see, for example, Green and Wuts Protective Groups in Organic Synthesis. John Wiley and Sons, New York, 1999). Hydroxyl or amino groups may be protected with any hydroxyl or amino protecting group. The amino protecting groups may be removed by conventional techniques. For example, acyl groups, such as alkanoyl, alkoxycarbonyl and aroyl groups, may be removed by solvolysis, e.g., by hydrolysis under acidic or basic conditions. Arylmethoxycarbonyl groups (e.g., benzyloxycarbonyl) may be cleaved by hydrogenolysis in the presence of a catalyst such as palladium-on-charcoal.

The present invention also encompasses prodrugs of the compounds of formula I, i.e., compounds which release an active parent drug according to formula I in vivo when administered to a mammalian subject. A prodrug is a pharmacologically active or more typically an inactive compound that is converted into a pharmacologically active agent by a metabolic transformation. Prodrugs of a compound of formula I are prepared by modifying functional groups present in the compound of formula I in such a way that the modifications may be cleaved in vivo to release the parent compound. In vivo, a prodrug readily undergoes chemical changes under physiological conditions (e.g., are acted on by naturally occurring enzyme(s)) resulting in liberation of the pharmacologically active agent. Prodrugs include compounds of formula I wherein a hydroxy, amino, or carboxy group of a formula I compound is bonded to any group that may be cleaved in vivo to regenerate the free hydroxyl, amino or carboxy group, respectively. Examples of prodrugs include, but are not limited to esters (e.g., acetate, formate, and benzoate derivatives) of compounds of formula I or any other derivative which upon being brought to the physiological pH or through enzyme action is converted to the active parent drug. Conventional procedures for the selection and preparation of suitable prodrug derivatives are described in the art (see, for example, Bundgaard. Design of Prodrugs. Elsevier, 1985).

Prodrugs may be administered in the same manner as the active ingredient to which they convert or they may be delivered in a reservoir form, e.g., a transdermal patch or other reservoir which is adapted to permit (by provision of an enzyme or other appropriate reagent) conversion of a prodrug to the active ingredient slowly over time, and delivery of the active ingredient to the patient.

Unless specifically indicated, the term “active ingredient” is to be understood as referring to a compound of formula I as defined herein.

The present invention also encompasses metabolites. “Metabolite” of a compound disclosed herein is a derivative of a compound which is formed when the compound is metabolised. The term “active metabolite” refers to a biologically active derivative of a compound which is formed when the compound is metabolised. The term “metabolised” refers to the sum of the processes by which a particular substance is changed in the living body. In brief, all compounds present in the body are manipulated by enzymes within the body in order to derive energy and/or to remove them from the body. Specific enzymes produce specific structural alterations to the compound. For example, cytochrome P450 catalyses a variety of oxidative and reductive reactions while uridine diphosphate glucuronyltransferases catalyse the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996), pages 11-17.

Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

### Pharmaceutical Compositions Comprising a Compound of Formula I

For use in the methods of the invention, a compound of formula I may be administered as the bulk substance, e.g., a pure compound. More preferably, a compound of Formula I is present in a pharmaceutical formulation, e.g., wherein the active agent is in admixture with a pharmaceutically acceptable carrier, selected with regard to the intended route of administration and standard pharmaceutical practice.

Accordingly, in one aspect, the present invention provides a pharmaceutical composition comprising at least one compound of formula I, or a pharmaceutically acceptable derivative (e.g., a salt or solvate) thereof, and, optionally, a pharmaceutically acceptable carrier. In particular, the invention provides a pharmaceutical composition comprising a therapeutically effective amount of at least one compound of formula I, or a pharmaceutically acceptable derivative thereof, and, optionally, a pharmaceutically acceptable carrier.

For the methods of the invention, a compound of formula I may be used in combination with other therapies and/or active agents. Accordingly, the present invention provides, in a further aspect, a pharmaceutical composition comprising at least one compound of formula I, or a pharmaceutically acceptable derivative thereof, a second active agent, and, optionally a pharmaceutically acceptable carrier.

When combined in a single formulation it will be appreciated that the two compounds must be stable and compatible with each other and the other components of the formulation.

The phrase “pharmaceutically acceptable” refers to molecular entities and compositions that are generally regarded as safe for administration to a mammal, e.g., a human being. In particular, pharmaceutically acceptable carriers used in the pharmaceutical compositions of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The compounds of the invention may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example see International Patent Application No. WO 02/00196 (SmithKline Beecham).

### Routes of Administration and Unit Dosage Forms

The routes for administration (delivery) include, but are not limited to, one or more of oral (e.g., as a tablet, capsule, or as an ingestible solution), topical, mucosal (e.g., as a nasal spray or aerosol for inhalation), nasal, parenteral (e.g., by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual routes of administration.

Accordingly, compositions of the invention include those in a form especially formulated for, e.g., parenteral, oral, buccal, rectal, topical, implant, ophthalmic, nasal or genito-urinary use. In preferred embodiments, the pharmaceutical compositions of the invention are formulated to be suitable for oral delivery.

There may be different composition/formulation requirements depending on the different delivery systems. It is to be understood that not all of the compounds need to be administered by the same route. Likewise, if a composition comprises more than one active component, each component may be administered by a different route. By way of example, the pharmaceutical composition of the present invention may be formulated to be delivered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the formulation may be designed to be delivered by multiple routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile. In certain embodiments, a compound of formula I, or a composition thereof, may be coated with an enteric coating layer. The enteric coating layer material may be dispersed or dissolved in either water or in a suitable organic solvent. As enteric coating layer polymers, one or more, separately or in combination, of the following can be used; e.g., solutions or dispersions of methacrylic acid copolymers, cellulose acetate phthalate, cellulose acetate butyrate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating layer polymer(s). For environmental reasons, an aqueous coating process may be preferred. In such aqueous processes methacrylic acid copolymers are most preferred.

Pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intravenously, intramuscularly or subcutaneously. For buccal or sublingual administration the compositions may be administered in the form of tablets or lozenges, which can be formulated in a conventional manner.

Where the composition of the invention is to be administered parenteral, such administration includes one or more intravenous, intraarterial, intraperitoneal, intrathecal, intraventricular, intraurethral, intrasternal, intracranial, intramuscular or subcutaneous administration and/or infusion techniques.

Pharmaceutical compositions of the present invention can be administered parenterally, e.g., by infusion or injection. Pharmaceutical compositions suitable for injection or infusion may be in the form of a sterile aqueous solution, a dispersion or a sterile powder that contains the active ingredient, adjusted, if necessary, for preparation of such a sterile solution or dispersion suitable for infusion or injection. This preparation may optionally be encapsulated into liposomes. The final preparation should be sterile, liquid, and stable under production and storage conditions. To improve storage stability, such preparations may also contain a preservative to prevent the growth of microorganisms. Anti-microbial action of micro-organisms can be provided by the addition of various antibacterial and antifungal agents, e.g., paraben, chlorobutanol, or acsorbic acid. In many cases isotonic substances are recommended, e.g., sugars, buffers and sodium chloride to assure osmotic pressure similar to those of body fluids, particularly blood. Prolonged absorption of such injectable mixtures can be achieved by introduction of absorption-delaying agents, such as aluminium monostearate or gelatin.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

For parenteral administration, the compound is preferably in the form of a sterile aqueous solution which may contain other substances, such as, for example, sufficient salts or glucose to make the solution isotonic with blood. Aqueous solutions are preferably buffered (preferably to a pH of from 3 to 9), if necessary. The preparation of suitable parenteral formulations under sterile conditions is readily accomplished by standard pharmaceutical techniques well-known to those skilled in the art.

Sterile injectable solutions can be prepared by mixing a compound of formula I with an appropriate solvent and one or more of the aforementioned carriers, followed by sterile filtering. In the case of sterile powders suitable for use in the preparation of sterile injectable solutions, preferable preparation methods include drying in vacuum and lyophilization, which provide powdery mixtures of the aldosterone receptor antagonists and desired excipients for subsequent preparation of sterile solutions.

The compounds according to the invention may be formulated for use in human or veterinary medicine by injection (e.g., by intravenous bolus injection or infusion or via intramuscular, subcutaneous or intrathecal routes) and may be presented in unit dose form, in ampules, or other unit-dose containers, or in multi-dose containers, if necessary with an added preservative. Compositions for injection may be in the form of suspensions, solutions, or emulsions, in oily or aqueous vehicles, and may contain formulatory agents such as suspending, stabilizing, solubilizing and/or dispersing agents. Alternatively the active ingredient may be in sterile powder form for reconstitution with a suitable vehicle, e.g., sterile, pyrogen-free water, before use.

The compounds of the invention can be administered (e.g., orally or topically) in the form of tablets, capsules, ovules, elixirs, solutions or suspensions, which may contain flavoring or coloring agents, for immediate-, delayed-, modified-, sustained-, pulsed- or controlled-release applications.

The compounds of the invention may also be presented for human or veterinary use in a form suitable for oral or buccal administration, for example in the form of solutions, gels, syrups, mouth washes or suspensions, or a dry powder for constitution with water or other suitable vehicle before use, optionally with flavoring and coloring agents. Solid compositions such as tablets, capsules, lozenges, pastilles, pills, boluses, powder, pastes, granules, bullets or premix preparations may also be used. Solid and liquid compositions for oral use may be prepared according to methods well-known in the art. Such compositions may also contain one or more pharmaceutically acceptable carriers and excipients which may be in solid or liquid form.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

The compositions may be administered orally, in the form of rapid or controlled release tablets, microparticles, mini tablets, capsules, sachets, and oral solutions or suspensions, or powders for the preparation thereof. In addition to the new solid-state forms of pantoprazole of the present invention as the active substance, oral preparations may optionally include various standard pharmaceutical carriers and excipients, such as binders, fillers, buffers, lubricants, glidants, dyes, disintegrants, odorants, sweeteners, surfactants, mold release agents, antiadhesive agents and coatings. Some excipients may have multiple roles in the compositions, e.g., act as both binders and disintegrants.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

The compounds of the invention may also, for example, be formulated as suppositories e.g., containing conventional suppository bases for use in human or veterinary medicine or as pessaries e.g., containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g., eye, ear or nose drops) or pour-ons.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax, sorbitan monostearate, a polyethylene glycol, liquid paraffin, polysorbate 60, cetyl esters wax, cetearyl alcohol, 2-octyldodecanol, benzyl alcohol, and water. Such compositions may also contain other pharmaceutically acceptable excipients, such as polymers, oils, liquid carriers, surfactants, buffers, preservatives, stabilizers, antioxidants, moisturizers, emollients, colorants, and odorants.

Examples of pharmaceutically acceptable polymers suitable for such topical compositions include, but are not limited to, acrylic polymers; cellulose derivatives, such as carboxymethylcellulose sodium, methylcellulose or hydroxypropylcellulose; natural polymers, such as alginates, tragacanth, pectin, xanthan and cytosan.

Examples of suitable pharmaceutically acceptable oils which are so useful include but are not limited to, mineral oils, silicone oils, fatty acids, alcohols, and glycols.

Examples of suitable pharmaceutically acceptable liquid carriers include, but are not limited to, water, alcohols or glycols such as ethanol, isopropanol, propylene glycol, hexylene glycol, glycerol and polyethylene glycol, or mixtures thereof in which the pseudopolymorph is dissolved or dispersed, optionally with the addition of non-toxic anionic, cationic or non-ionic surfactants, and inorganic or organic buffers.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyanisole.

Suitable examples of pharmaceutically acceptable moisturizers include, but are not limited to, glycerine, sorbitol, urea and polyethylene glycol.

Suitable examples of pharmaceutically acceptable emollients include, but are not limited to, mineral oils, isopropyl myristate, and isopropyl palmitate.

The compounds may also be dermally or transdermally administered, for example, by use of a skin patch.

For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride.

As indicated, the compound of the present invention can be administered intranasally or by inhalation and is conveniently delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant, e.g., sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebulizer.

The pharmaceutical compositions of the invention may contain from 0.01 to 99% weight per volume of the active material. For topical administration, for example, the composition will generally contain from 0.01-10%, more preferably 0.01-1% of the active material.

The active agents can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The pharmaceutical composition or unit dosage forms comprising an effective amount of the present invention may be administered to an animal, preferably a human, in need of treatment of neuromuscular dysfunction of the lower urinary tract described by E. J. McGuire in “Campbell's UROLOGY”, 5^{th} Ed., 616-638, 1986, W.B. Saunders Company, and patients affected by any physiological dysfunction related to impairment of 5-HT_{1A} receptor function. Such dysfunctions include, without limitation, central-nervous-system disorders such as depression, anxiety, eating disorders, sexual dysfunction, addiction and related problems.

As used herein, the term “effective amount” refers to an amount that results in measurable amelioration of at least one symptom or parameter of a specific disorder. In a preferred embodiment, the compound treats disorders of the urinary tract, such as urinary urgency, overactive bladder, increased urinary frequency, reduced urinary compliance (reduced bladder storage capacity), cystitis (including interstitial cystitis), incontinence, urine leakage, enuresis, dysuria, urinary hesitancy and difficulty in emptying the bladder, or central nervous system disorders due to serotonergic dysfunction (such as anxiety, depression, hypertension, sleep/wake cycle disorders, feeding behaviour, sexual function and cognition disorders in mammals (particularly a human) associated to stroke, injury, dementia and due to neurological development, disorders from hyperactivity related to an attention deficit (ADHD), drug addiction, drug withdrawal, irritable bowel syndrome.

The pharmaceutical composition or unit dosage form of the present invention may be administered according to a dosage and administration regimen defined by routine testing in the light of the guidelines given above in order to obtain optimal activity while minimizing toxicity or side effects for a particular patient. However, such fine tuning of the therapeutic regimen is routine in the light of the guidelines given herein.

The dosage of the active agents of the present invention may vary according to a variety of factors such as underlying disease conditions, the individual's condition, weight, sex and age, and the mode of administration. An effective amount for treating a disorder can easily be determined by empirical methods known to those of ordinary skill in the art, for example by establishing a matrix of dosages and frequencies of administration and comparing a group of experimental units or subjects at each point in the matrix. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter of urinary tract disorders is within the scope of the invention. Clinically significant attenuation or amelioration means perceptible to the patient and/or to the physician.

For example, a single patient may suffer from several symptoms of dysuria simultaneously, such as, for example, urgency and excessive frequency of urination or both, and these may be reduced using the methods of the present invention. In the case of incontinence, any reduction in the frequency or volume of unwanted passage of urine is considered a beneficial effect of the present method of treatment.

The amount of the agent to be administered can range between about 0.01 and about 25 mg/kg/day, preferably between about 0.1 and about 10 mg/kg/day and most preferably between 0.2 and about 5 mg/kg/day. It will be understood that the pharmaceutical formulations of the present invention need not necessarily contain the entire amount of the agent that is effective in treating the disorder, as such effective amounts can be reached by administration of a plurality of doses of such pharmaceutical formulations.

In a preferred embodiment of the present invention, the compounds of Formula I are formulated in capsules or tablets, preferably containing 10 to 200 mg of the compounds of the invention, and are preferably administered to a patient at a total daily dose of 10 to 300 mg, preferably 20 to 150 mg and most preferably about 50 mg, for relief of urinary incontinence and other dysfunctions, wherein the compounds of Formula I are 5-HT_{1A} receptor ligands. Most preferably, the compounds of Formula I are 5-HT_{1A} receptor antagonists or inverse agonists.

A pharmaceutical composition for parenteral administration contains from about 0.01% to about 100% by weight of the active agents of the present invention, based upon 100% weight of total pharmaceutical composition.

Generally, transdermal dosage forms contain from about 0.01% to about 100% by weight of the active agents versus 100% total weight of the dosage form.

The pharmaceutical composition or unit dosage form may be administered in a single daily dose, or the total daily dosage may be administered in divided doses. In addition, co-administration or sequential administration of another compound for the treatment of the disorder may be desirable. For example, the compounds of the invention may be administered in combination with known antimuscarinic drugs such as, for example, oxybutynin, tolterodine, darifenacin and temiverine. Analogously, the compounds of the invention may be administered with α₁-adrenergic antagonists such as, for example, prazosin, doxazosin, terazosin, alfuzosin and tamsulosin for the therapy of the lower urinary tract symptoms.

For combination treatment where the compounds are in separate dosage formulations, the compounds can be administered concurrently, or each can be administered at staggered intervals. For example, the compound of the invention may be administered in the morning and the antimuscarinic compound may be administered in the evening, or vice versa. Additional compounds may be administered at specific intervals too. The order of administration will depend upon a variety of factors including age, weight, sex and medical condition of the patient; the severity and etiology of the disorders to be treated, the route of administration, the renal and hepatic function of the patient, the treatment history of the patient, and the responsiveness of the patient. Determination of the order of administration may be fine-tuned and such fine-tuning is routine in the light of the guidelines given herein.

### Methods of Treatment

Without wishing to be bound by theory, it is believed that administration of 5-HT_{1A} receptor antagonists and/or inverse agonists prevents unwanted activity of the sacral reflex and/or cortical mechanisms that control micturition. Thus, it is contemplated that a wide range of neuromuscular dysfunctions of the lower urinary tract can be treated using the compounds of the present invention, including without limitation dysuria, incontinence and enuresis (overactive bladder). Dysuria includes urinary frequency, nocturia, urgency, reduced urinary compliance (reduced bladder storage capacity), difficulty in emptying the bladder, i.e., a suboptimal volume of urine is expelled during micturition. Incontinence syndromes include stress incontinence, urgency incontinence and enuresis incontinence, as well as mixed forms of incontinence. Enuresis refers to the involuntary passage of urine at night or during sleep.

The present invention also contemplates the administration of the compounds of Formula I in combination other classes of agents known to be useful for treatment of lower urinary tract disorders. In a preferred embodiment, compounds of the invention are administered with antimuscarinics. Antimuscarinics are known to be useful in the treatment of, for example, dysfunction of the lower urinary tract, including, urinary bladder dysfunction. Antimuscarinics have been used to reduce unstable detrusor contractions and increase functional bladder capacity and are widely used in the treatment of urge, sensory, and motor urge incontinence (See Ferguson et al., Urinary Bladder Function And Drug Development TIPS 17:161-165, 1996; Andersson, Advances In The Pharmacological Control Of The Bladder Experimental Physiology 84:195-213, 1999; and Andersson, The Overactive Bladder: Pharmacological Basis Of Drug Treatment Urology 50:74-89, 1997). Antimuscarinics are also useful for treatment of overactive bladder, see K E Andersson, Lancet Neurology, 3(1) 46-53, 2004; and Garely et al, Expert. Opin. Pharmacother 3:827-833, 2002. Without wishing to be bound by theory, compounds of Formula I and antimuscarinics contribute to treatment of lower urinary tract disorder through different mechanisms. Thus, compounds of Formula I are believed to achieve their action by binding to 5HT_{1A} receptors in the CNS; antimuscarinics are believed to achieve their effect by binding to receptors in the bladder. Thus, co-administration of a compound of Formula I and an antimuscarinic offers benefits to administering each alone, e.g., the opportunity to lower the administered amount of each single drug, thus lowering the risks of side effects associated with administration of the compounds.

The compounds of the present invention may also be useful for the treatment of central nervous system disorders due to serotonergic dysfunction. For example, the compounds of the present invention may be used in the treatment of prophylaxis of Alzheimer's disease (AD). AD is a neurodegenerative disorder characterized by multiple deficits in neurotransmitter function. Although many studies have focused upon the cholinergic hypothesis, it is now apparent that not all patients can be characterized by deficits in this system alone, as shown by the moderate efficacy produced by acetylcholinesterase inhibitors (Bliss et al, Nature (Lond) 361:31-39, 1993). Recent research suggests that glutamatergic deficits may occur before those observed in the cholinergic system (Id.). Furthermore, the serotonergic system may be hyperactive in AD as a result of the enhanced turnover of serotonin (Kowall et al, Ann Neurol 29:162-167, 1991), which ultimately would reduce the firing of the cortical pathways through stimulation of 5-HT_{1A} somato-dendritic receptors. The enhanced serotonergic turnover observed in AD has suggested that 5-HT_{1A} receptor antagonists may be effective in treating the cognitive loss associated with AD. Bowen et al., (Trends Neurosci 17:149-150, 1994).

A role for 5HT_{1A} in AD etiology is also suggested by the observations that N-Methyl-D-aspartate (NMDA)-induced glutamate release from pyramidal neurons is potentiated by 5-HT_{1A} receptor antagonists (Dijk et al., Br J Pharmacol 115:1169-1174, 1995) and that the 5-HT_{1A} antagonist WAY-100635 can alleviate cognitive deficits induced by both glutamatergic and cholinergic dysfunction in primates (Harder et al., Psychopharmacology 127:245-254, 1996; Harder and Ridley, Neuropharmacology 39:547-552, 2000). The blockade of 5-HT_{1A} receptors also enhances glutamate release from rat hippocampal slices (Van den Hooff et al, Eur J Pharmacol 196:291-298, 1991). Other data suggest that 5-HT_{1A} receptor antagonists can inhibit the tonic hyperpolarizing action of serotonin on pyramidal neurons, in both the cortex and hippocampus (Araneda et al., Neuroscience 40:399-412, 1991; Van den Hooff et al., Br J Pharmacol 106:893-899, 1992). In this way, 5HT_{1A} receptor antagonists enhanced glutamatergic neurotransmission and signalling.

Recently, a novel selective 5-HT_{1A} antagonist, lecozotan, has been reported to be active in a variety of models related to cognition enhancements (Schechter et al., J. Pharmacol. Exp. Therap. 314:1274-1289, 2005). Lecozotan significantly potentiated the potassium chloride-stimulated release of glutamate and acetylcholine in the dentate gyrus of the hippocampus. In drug discrimination studies, lecozotan (0.01-1 mg/kg i.m.) did not substitute for 8-OH-DPAT and produced a dose-related blockade of the 5-HT_{1A} agonist discriminative stimulus cue. In aged rhesus monkeys, lecozotan produced a significant improvement in task performance efficiency at the dose of 1 mg/kg p.o. Learning deficits induced by the glutamatergic antagonist MK-801 and by specific cholinergic lesions of the hippocampus (assessed by visual spatial discrimination) were reversed by lecozotan (2 mg/kg i.m.) in marmosets. These observations further support a role for 5HT_{1A} antagonists in treatment of AD.

These results confirm the potential for 5-HT_{1A} antagonists in the care of the biochemical pathologies underlying cognitive loss in AD. Thus, without wishing to be bound by any particular theory, the compounds of Formula I may improve cognition by removing the inhibitory effects of endogenous serotonin on pyramidal neurons and thus enhance glutamatergic activation and ensuing signal transduction.

### Synthesis of the Compounds of the Invention

It will be appreciated by those skilled in the art that it may be desirable to use protected derivatives of intermediates used in the preparation of the compounds of formula I. Protection and deprotection of functional groups may be performed by methods known in the art. Hydroxyl or amino groups may be protected with any hydroxyl or amino protecting group (for example, as described in Green and Wuts, Protective Groups in Organic Synthesis. John Wiley and Sons, New York, 1999). The protecting groups may be removed by conventional techniques. For example, acyl groups (such as alkanoyl, alkoxycarbonyl and aryloyl groups) may be removed by solvolysis (e.g., by hydrolysis under acidic or basic conditions). Arylmethoxycarbonyl groups (e.g., benzyloxycarbonyl) may be cleaved by hydrogenolysis in the presence of a catalyst such as palladium-on-carbon.

The synthesis of a target compound is completed by removing any protecting groups, which are present in the penultimate intermediate, using standard techniques, which are well-known to those skilled in the art. The deprotected final product is then purified, as necessary, using standard techniques, such as silica gel chromatography, HPLC on silica gel and the like, or by recrystallization.

The compounds of the invention are generally prepared according to the following schemes:

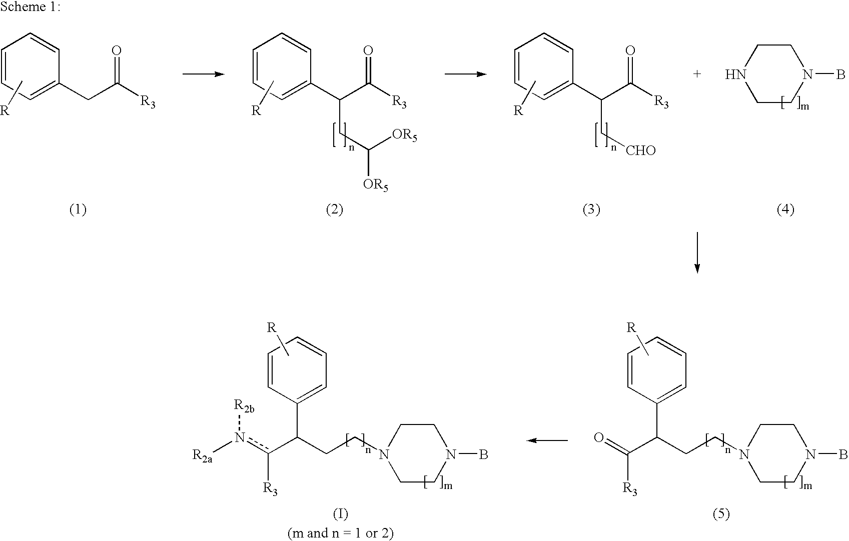

Groups B are the same as groups A-R₄, and R, R₂ₐ, R_{2b}, and R₃ are as given in the general Formula I. R₅ is a lower alkyl group, or the R₅ substituents may be joined to form a cyclic acetal.

In Scheme 1, starting material (1) is treated with a base, preferably potassium tert-butoxide, followed by alkylation of the resultant enolate with 2-bromoacetaldehyde dialkyl acetal or other carbonyl protected 2-haloacetaldehyde (e.g., the R₅ alkyl groups can also be joined in a cycle to give a dioxolane or dioxane ring). These reactions will give the compound (2), wherein n=1. Other alternative and appropriate bases to carry out the condensation include lithium amides, sodium hydride, sodium hydroxide, potassium hydroxide, potassium carbonate, cesium carbonate and the like. The reaction may also be carried out in the presence of phase transfer catalysts.

The reaction is preferably carried out in a solvent such as dimethyl sulfoxide or toluene or tetrahydrofuran at a temperature of from about 0° C. to about the boiling point of the reaction solvent.

The use of 3-bromopropionaldehyde dialkyl acetal or other carbonyl protected 3-halopropionaldehydes provides for a compound for Formula (2) wherein n=2, by following the same reaction conditions described above in Scheme 1, wherein n=1.

Treatment of the acetal (2) with an acid, such as hydrochloric acid, p-toluenesulfonic acid or trifluoroacetic acid in a suitable organic solvent, provides aldehyde (3) upon removal of the acetal moiety. Generally, the reaction is conducted in a polar solvent, such a mixture of aqueous acid and acetone or tetrahydrofuran, at temperatures of from about 5° to about 75° C., preferably at ambient temperature. In one embodiment, a preferred method consists of carrying out the reaction in a mixture of aqueous trifluoroacetic acid in a chlorinated solvent (e.g. 1,2-dichloroethane) at ambient temperature.

Aldehyde (3) is coupled with the desired substituted piperazine or homopiperazine (4) by, for example, a reductive amination procedure to prepare coupling product (5). The reaction is preferably carried out at ambient temperature in a non-reactive solvent such as 1,2-dichloroethane, methylene chloride or chloroform in the presence of, for example, sodium triacetoxyborohydride and, if needed, a protic acid (preferably acetic acid) (see e.g., A. F. Abdel-Magid, et al., J. Org. Chem., 61, 3849 (1996)). Alternatively the reaction can be performed in a protic solvent (e.g., methanol) with the aid of sodium cyanoborohydride optionally in the presence of molecular sieves and in the presence of the proper amount of an acid (e.g. ethanolic or methanolic hydrochloric acid).

For the compounds of Formula I, wherein [symbol] is a double bond, compound 5 is reacted with a primary amine in the presence of a dehydrating agent (e.g., MgSO4 or molecular sieves) or under Dean-Stark conditions to afford an imine upon removal of water.

For the compounds of Formula I, wherein [symbol] is a single bond, a subsequent reductive amination procedure of (5) to an amino derivative of (I) is readily accomplished as described above or, alternatively, e.g. in the case of N—O derivatives, the preliminary formation of the imino (C═N) derivative (e.g. oxime), which are also compounds of Formula I, followed by the proper reduction of the double bond using a reducing agent chosen from an amino-boron complex (pyridine-borane, triethylamine-borane or the like), sodium cyanoborohydride or, diisobutylaluminum hydride or other aluminum or boron hydride, or other appropriate reagents for imine reduction. All these reagents are well known to those skilled in the art and often used to prepare the nitrogen or N—O compounds of Formula (I). The reduction of the C═N double bond is preferably conducted in an organic solvent such as methanol, methylene chloride or tetrahydrofuran, or without any solvent at from about −20° C. to about ambient temperature.

Compounds (I) can also be conveniently prepared by carrying out a modified version of the Leuckart reaction conducted under focused microwave irradiation (Loupy, A. et al. Tetrahedron Letters 1996, 37, 8177). The reaction is performed in a microwave oven (CEM or Personal Chemistry) in formic acid-formamide. Using the Leuckart reaction, the compounds of Formula (I) obtained are those with —N(R₂ₐ)(R_{2b}) equal to NHCHO. These formamides are suitable to be hydrolized to the corresponding NH₂ derivatives upon treatment with acid or base. Alternatively, the formamides are reduced with, e.g. a borane complex (borane-dimethyl sulfide, aminoboranes and others) to the corresponding NHCH₃ derivatives.

All the procedures related to this Leuckart modified method are described in the foregoing exemplary methods.

The compounds of Formula (I) can be further derivatized to afford the other Formula I compounds of the invention. The derivatives can be easily synthesized by procedures well known to those skilled in the art, including acylation reactions, for example, direct acylation using an aroyl, heteroaroyl, or alkanoyl halogenide, or a haloformamide or halothioformamide, or an alkyl haloformate, or halothioformate or aryl(alkyl)iso(thio)cyanate, in the presence of a proper base (e.g., triethylamine or the like); or by an indirect procedure which consists of reacting the compounds of Formula (I) bearing —NHR₂ₐ groups with phosgene, thiophosgene, or a less toxic substitute e.g., triphosgene, carbonyldiimidazole, thiocarbonyldiimidazole or the like, to form the intermediate haloformamides (thioformamides), imidazolides (thioimidazolides), isocyanates, or isothiocyanates of Formula (I) and further reacting these species with alcohols, alkoxides or amines.

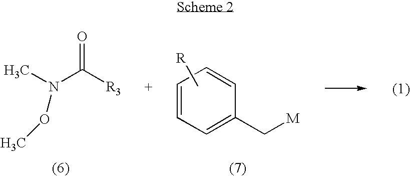

As shown in Scheme 2, starting material (1), if not commercially commercially available, can be prepared by coupling the appropriate Weinreb amide (6) (See, Nahm and Weinreb, Tetrahedron Lett., 22, 3815, (1981)) with benzyl derivative (7), where M is a metallic salt, such as lithium or magnesium halide. The reaction is preferably conducted under an inert atmosphere (e.g., under nitrogen) in an aprotic solvent, such as tetrahydrofuran, at ambient or lower temperatures (e.g., from 0° C. to −78° C.).

Alternatively, an ester of structure R₃C(O)O-alkyl can be treated with a substituted benzylmagnesium chloride or benzylmagnesium bromide to provide the ketone of structure (1).

A preferred way for the synthesis of (1) is the palladium catalyzed coupling of an acyl halide with a compound (7), wherein M is a Zn halide.

More specifically, the compounds of formula (5) can be prepared following the procedure described in Scheme 3. All substituents, unless otherwise indicated, are as defined previously. The reagents and starting materials are readily available to one of ordinary skill in the art.

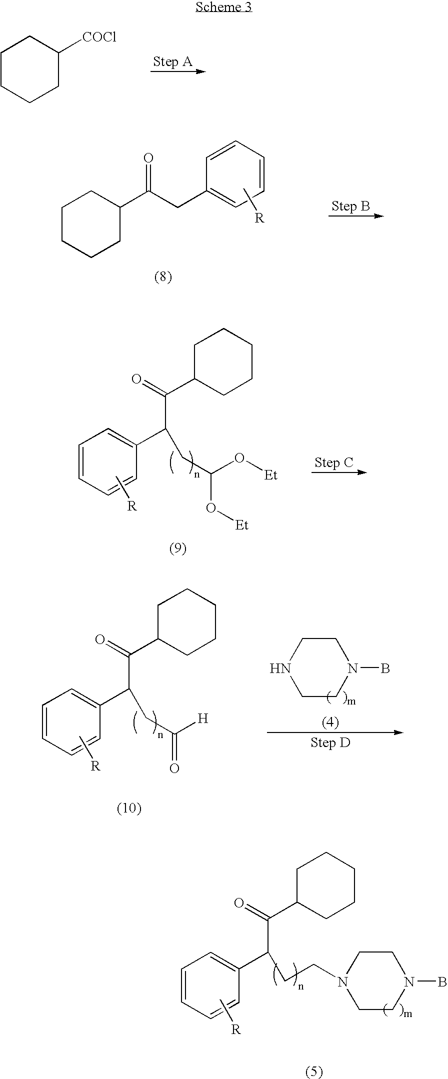

In Scheme 3, step A, for example, cyclohexanecarbonyl chloride is added to a mixture of the appropriate benzylzinc chloride or bromide and a suitable palladium catalyst (e.g., (triphenylphosphine)palladium(II) dichloride) at about 0° C. in a solvent such as tetrahydrofuran. Afterwards, stirring is continued at ambient temperature for 4-24 h. Then, the reaction is quenched with, for example, an aqueous saturated solution of ammonium chloride. Ketone (8) can be purified by techniques well known in the art, such as flash chromatography on silica gel with a suitable eluent, such as ethyl acetate/hexane. Alternatively, the crude ketone (8) can be carried on to step B.

In Scheme 3, step B, ketone (8) is alkylated with, for example, bromoacetaldehyde diethyl acetal under conditions well known in the art to provide alkylated compound (9). For example, ketone (8) is dissolved in a suitable organic solvent, such as dimethyl sulfoxide or toluene and treated with a slight excess of a suitable base, such as potassium tert-butoxide (see also, Scheme 1). The reaction is stirred for about 15 to 30 minutes at a temperature of from about 0° C. to the reflux temperature of the solvent and bromoacetaldehyde diethyl acetal is added dropwise to the reaction. One of ordinary skill in the art would readily appreciate that bromoacetaldehyde dimethyl acetal, bromoacetaldehyde ethylene acetal (i.e., a dioxolane) and the like may be used in place of the corresponding diethyl acetal.

In Scheme 3, step C, compound (9) is hydrolyzed under acidic conditions to provide aldehyde (10) in a manner analogous to the procedure described for Scheme 1. Specifically, for instance, compound (9) is dissolved in a suitable organic solvent, such as dichloromethane and treated with a suitable acid, such as aqueous trifluoroacetic acid. The reaction mixture is stirred for about 1 to 6 hours at about room temperature. The reaction mixture is then diluted with the same solvent, washed with brine, the organic layer is separated, dried over anhydrous sodium sulfate, filtered and concentrated under vacuum to provide aldehyde (10). Aldehyde (10) can be purified by techniques well known in the art, such as flash chromatography on silica gel with a suitable eluent, such as ethyl acetate/hexane. Alternatively, crude aldehyde (10) can be used directly in step D.

In Scheme 3, step D, aldehyde (10) is reductively aminated, under conditions well known in the art, with piperazine (4) to provide the ketone (5) in a manner analogous to the procedure described in Scheme I. Specifically, for example, aldehyde (10) is dissolved in a suitable organic solvent, such as methylene chloride, and to this solution is added about 1.05 or more equivalents of piperazine (4). Acetic acid may optionally be added to aid in dissolution of the piperazine. Then about 1.4 to 1.5 equivalents of sodium triacetoxyborohydride is added and the reaction is stirred at room temperature for about 3 to 5 hours. The reaction is then quenched by addition of a suitable base, such as aqueous sodium carbonate or hydroxide to provide a pH of about 8 to about 12. The quenched reaction is then extracted with a suitable organic solvent, such as methylene chloride. The organic extracts are combined, washed with brine, dried, filtered and concentrated under vacuum to provide the compound of formula (5). This material can then be purified by techniques well known in the art, such as flash chromatography on silica gel with a suitable eluent, such as ethyl acetate/petroleum ether or hexane.

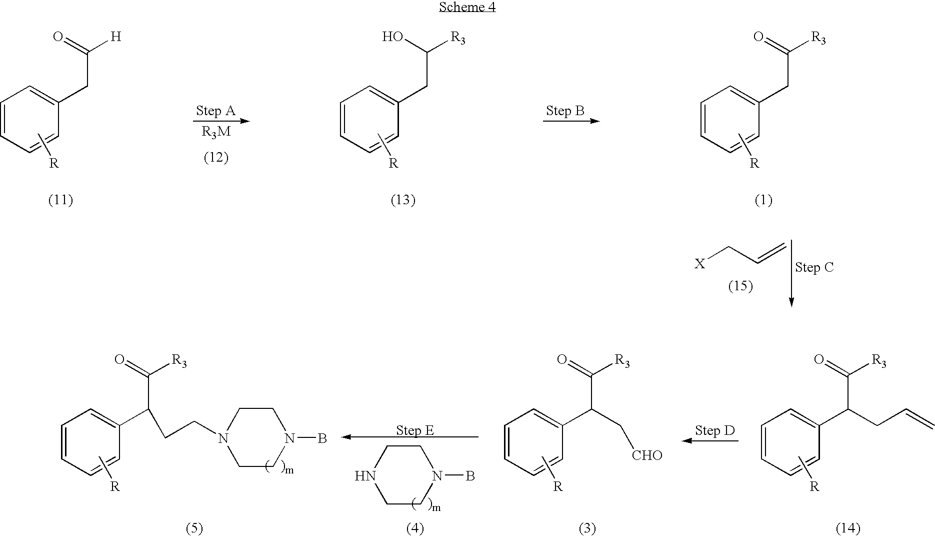

Alternatively, compounds of structure (5) can be prepared following the procedure shown in Scheme 4. All substituents, unless otherwise indicated, are defined previously. The reagents and starting materials are readily available to one of ordinary skill in the art.

In Scheme 4, step A, aldehyde (11) is combined with a suitable organometallic reagent (12) under conditions well known in the art to provide alcohol (13). Examples of suitable organometallic reagents include Grignard Reagents, alkyl lithium reagents, alkyl zinc reagents, and the like. Grignard Reagents are preferred. For examples of typical Grignard Reagents and reaction conditions, see J. March, “Advanced Organic Chemistry Reactions, Mechanisms, and Structure”, 2nd Edition, McGraw-Hill, pages 836-841 (1977). Specifically, aldehyde (11) is dissolved in a suitable organic solvent, such as tetrahydrofuran or toluene, cooled to about −5° C. and treated with about 1.1 to 1.2 equivalents of a Grignard reagent of formula (12) wherein M is MgCl or MgBr. The reaction is stirred for about 0.5 to 6 hours, then quenched, and alcohol (13) is isolated.

In Scheme 4, step B, alcohol (13) is oxidized under standard conditions well know in the art, such as those described by J. March, “Advanced Organic Chemistry: Reactions, Mechanisms, and Structure”, 2nd Edition, McGraw-Hill, pages 1082-1084 (1977), to provide ketone (1). (Ketone (1) is the starting material used in Scheme 1 above.)

The oxidation can also be performed using standard Swern Oxidation conditions which are well known to one of ordinary skill in the art (Marx, Tidwell J. Org. Chem. 49, 788 (1984)) wherein the alcohol (13) is dissolved in a suitable organic solvent, such as methylene chloride, the solution cooled with a dry ice-acetone bath, and the alcohol treated with 2.5 to 3.0 equivalents of dimethyl sulfoxide. After stirring for about 30 minutes, the reaction is then treated with about 1.8 equivalents of P₂O₅. The reaction is stirred for about 3 hours and then, preferably, treated over about 30 minutes with about 3.5 equivalents of a suitable amine, such as triethylamine. The cooling bath is then removed and the reaction is stirred for about 8 to 16 hours. The ketone (1) is then isolated by standard extraction techniques well known in the art (e.g., flash chromatography on silica gel).

In Scheme 4, step C, ketone (1) is treated with a suitable base followed by addition of on allyl group (15), wherein X is a suitable leaving group, to provide allylated compound (14). For example, ketone (1) is combined with an excess of allylhalide (15) in a suitable organic solvent, such as tetrahydrofuran, and cooled with a dry ice acetone bath. Examples of suitable leaving groups are Cl, Br, I, tosylate, mesylate, and the like.

Preferred leaving groups are Cl and Br. About 1.1 equivalents of a suitable base is added and the reaction is allowed to stir for about 2 hours at room temperature. Examples of suitable bases are potassium tert-butoxide, sodium hydride, NaN(Si(CH₃)₃)₂, LDA, KN(Si(CH₃)₃)₂, NaNH₂, sodium ethoxide, sodium methoxide and the like. Potassium tert-butoxide is the preferred base. The reaction is then quenched with aqueous acid and the compound (14) is isolated by using well known procedures described herein (e.g., flash chromatography on silica gel).

In Scheme 4, step D, compound (14) is treated with a suitable oxidizing agent to provide aldehyde (3) (Aldehyde (3) is also prepared in Scheme 1). Examples of suitable oxidizing agents are ozone, NaIO₄/osmium catalyst, and the like. Ozone is the preferred oxidizing agent. Examples of suitable oxidizing reagents and conditions are described by J. March, “Advanced Organic Chemistry: Reactions, Mechanisms, and Structure”, 2nd Edition, McGraw-Hill, pages 1090-1096 (1977). For example, compound (14) is dissolved in a suitable organic solvent, such as methanol, a small amount of Sudan III is added, and the solution is cooled to about −20° C. Ozone is bubbled into the solution for about 4 hours until the pink color turns to a pale yellow color. Then, a reducing agent, such as Me₂S or tributylphosphine, is added. Solvent concentration provides the intermediate dimethyl acetal of aldehyde (3). This dimethyl acetal is readily hydrolyzed under standard acidic conditions to provide aldehyde (3). Alternatively, direct acidic work-up of the crude reaction mixture provides aldehyde (3). Alternatively, aldehyde (3) can be obtained directly by ozonolysis of (14) in a non-acetal forming solvent, such as methylene chloride.

In Scheme 4, step E, aldehyde (3) is reductively aminated under conditions analogous to those described above in Scheme 3, step D, to provide compound (5). (Compound 5 is also prepared in Scheme 1).

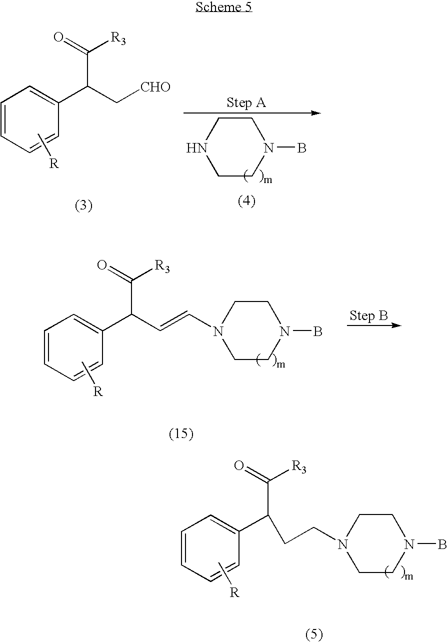

Scheme 5 provides an alternative synthesis for the preparation of ketone (5). All substituents, unless otherwise indicated, are as defined previously. The reagents and starting materials are readily available to one of ordinary skill in the art.

In Scheme 5, step A, aldehyde (3) is condensed with piperazine (4) under standard conditions to provide the enamine (15). For example, about 1.05 equivalents of aldehyde (3) dissolved in a suitable organic solvent, such as isopropyl acetate or isopropanol, are added to neat piperazine (4) free base. Additional organic solvent is added to produce a slurry and the reaction is stirred for about 1 to 2 hours. The enamine (15) is then isolated by standard techniques, such as, collection by filtration.

In Scheme 5, step B, the enamine (15) is hydrogenated under conditions well known to one of ordinary skill in the art to provide compound (5). For example, enamine (15) is combined with a suitable organic solvent, such as isopropyl alcohol and a catalytic amount of 5% palladium on carbon in a Parr bottle. The mixture is placed under 50 psi of hydrogen and shaken for about 2 days at room temperature. The slurry is then filtered to remove catalyst and the filtrate is concentrated to provide compound (5).

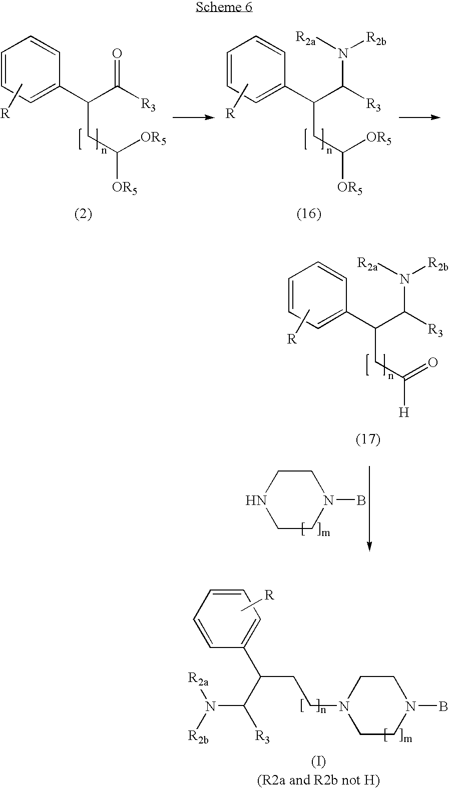

For the synthesis of compounds of Formula I where R₂ₐ and R_{2b} are not hydrogen, the alternative method given in Scheme 6 can be used. Intermediate ketone (2) is reacted with the same reductive amination methods used above in Scheme 1 for compound (5) affording intermediate (16), which is properly deprotected by a method chosen among those described above to afford aldehydes (17), which on turn can be reductively aminated to give Compound (I).

Alternatively, compounds of Formula I where R₂ₐ and R_{2b} are not a hydrogen atom, can be obtained by alkylating or acylating compounds of Formula I where R_{2b}=H with the same methods described above for alkylating compound (16), limiting this procedure to the alkylation with very reactive halogenides or mesylates/tosylates (e.g. benzyl bromides), preferably at room temperature.

The syntheses of the other compounds of Formula I which are not currently described in the general description above, are described in the foregoing exemplary methods.

In Schemes 1 and 6, the compounds of Formula I are obtained in syn/anti mixture of diastereomers with ratio depending on the structure of the reactants and the reaction condition used. The diastereomers can be separated by usual techniques known to those skilled in the art including fractional crystallization of the free bases or their salts or chromatographic techniques such as LC or flash chromatography. For both of the diastereomers, the (+) enantiomer of Formula I can be separated from the (−) enantiomer using techniques and procedures well known in the art, such as that described by J. Jacques, et al., “Enantiomers, Racemates, and Resolutions”, John Wiley and Sons, Inc., 1981. For example, chiral liquid chromatography with a suitable organic solvent, such as ethanol/acetonitrile and Chiralpak AD packing, 20 micron can also be utilized to effect separation of the enantiomers.

The free bases of formula I, their diastereomers or enantiomers can be converted to the corresponding pharmaceutically acceptable salts under standard conditions well known in the art. For example, the free base of formula I is dissolved in a suitable organic solvent, such as methanol, treated with, for example one equivalent of maleic or oxalic acid, one or two equivalents of hydrochloric acid or methanesulphonic acid, and then concentrated under vacuum to provide the corresponding pharmaceutically acceptable salt. The residue can then be purified by recrystallization from a suitable organic solvent or organic solvent mixture, such as methanol/diethyl ether.

The N-oxides of compounds of formula I can be synthesized by simple oxidation procedures well known to those skilled in the art. The oxidation procedure described by P. Brougham et al. (Synthesis, 1015-1017, 1987), allows the two nitrogens of the piperazine ring to be differentiated, enabling both the N-oxides and N,N′-dioxides to be obtained.

The following examples represent syntheses of the compounds of Formula I as described generally above. These examples are illustrative only and are not intended to limit the scope of the invention. The reagents and starting materials are readily available to one of ordinary skill in the art.

### EXAMPLE 1

### 1-Cyclohexyl-2-(2-fluorophenyl)-4-[4-(2-methoxyphenyl)-piperazin-1-yl]butan-1-one oxime

### 1-[4-Cyclohexyl-3-(2-fluorophenyl)-4-oxobutyl]-4-(2-methoxyphenyl)piperazine (Compound 1a)

This compound was prepared according to the procedure described in WO 03/106443 A1, for Compound 45d.

### 1-Cyclohexyl-2-(2-fluorophenyl)-4-[4-(2-methoxyphenyl)piperazin-1-yl]butan-1-one oxime

To a solution of 0.30 g of Compound 1a in 50 mL of EtOH—H₂O (4:1) was added 0.475 g of hydroxylamine hydrochloride and the solution was heated at reflux for 6 h. After that, additional 0.20 g of hydroxylamine hydrochloride was added and stirring at reflux was continued for 3 h. The solvent was evaporated in vacuo and the crude reaction mixture was taken up with H₂O (10 mL), basified with 1N NaOH and extracted with CH₂Cl₂ (2×10 mL). The organic layer was washed with brine, dried (Na₂SO₄) and evaporated to dryness to give a crude product, which was purified by flash chromatography (CH₂Cl₂-MeOH=95:5) affording the title compound (white solid, 0.30 g, 96.7%) as a (Z)/(E) or (E)/(Z)=8:2 mixture of diastereomers (LC-MS determination).

¹H-NMR (CDCl₃, δ): 0.80-1.80 (m, 10H), 1.80-2.10 (m, 1H), 2.12-2.90 (m, 7H), 2.90-3.30 (m, 5H), 3.86 (s, 3H), 4.00-4.20 (m, 0.8H), 4.70 (t, 0.2H), 6.80-7.50 (m, 8H), 8.00 (bs, 1H).

MS: [M+H]⁺=454.6.

### EXAMPLE 2

### 1-Cyclohexyl-2-(2-fluorophenyl)-4-[4-(2-methoxyphenyl)-piperazin-1-yl]butan-1-one O-methyloxime

To a solution of 0.10 g of Compound 1a in 17 mL of EtOH—H₂O (4:1) was added 0.19 g of O-methylhydroxylamine hydrochloride and the solution was heated at reflux for 24 h. During this time, 2 new aliquots of 0.095 g each of O-methylhydroxylamine hydrochloride (after 12 and 18 h) were added. The ethanol was evaporated, the aqueous layer was alkalinised with 1N NaOH and extracted with EtOAc (2×5 mL). The organic layer was washed, dried (Na₂SO₄) and evaporated to dryness to give a crude, which was purified by flash chromatography (petroleum ether-EtOAc-1.6 NNH₃ in MeOH=92:8:0.2) affording the title compound (0.044 g; 41.3%) as the pure Z(E) diastereomer (LC-MS, 1H-NMR determination)

¹H-NMR (CDCl₃, δ): 0.80-1.95 (m, 1H), 2.06-2.48 (m, 3H), 2.50-2.80 (m, 4H), 2.80-3.30 (m, 5H), 3.82, 3.93 (2×s, 6H), 4.10 (t, 1H), 6.80-7.28 (m, 7H), 7.35-7.70 (m, 1H).

MS: [M+H]⁺=468.6.

### EXAMPLE 3

### 1-Cyclohexyl-2-(2-fluorophenyl)-4-[4-(2-methoxyphenyl)-piperazin-1-yl]butan-1-one O-acetyl oxime

To a solution of 0.033 g of the Compound of Example 1 and 12.2 μL of TEA in 3 mL of CH₂Cl₂, stirred at 0° C. under a N₂ stream, was added 6.2 μL of acetyl chloride. The reaction mixture was stirred at r.t. for 8 h. After overnight resting, the mixture was poured into water (5 mL), basified (pH 8) with 10% aq. NaHCO₃ and extracted with CH₂Cl₂ (3×3 mL). The combined organic extracts were washed with brine, dried (Na₂SO₄) and evaporated to dryness to give a crude product (0.050 g), which was purified by flash chromatography (CH₂Cl₂-1.6 N NH₃ in MeOH=80:20) affording 0.013 g (36%) of the title product as a mixture (8:2) of E,Z (or Z,E) diastereomers.

¹H-NMR (CDCl₃, δ): 1.00-1.40 (m, 6H), 1.50-1.85 (m, 4H), 1.85-2.05 (m, 1H), 2.20-2.38 (m, 4H), 2.38-2.50 (m, 2H), 2.50-2.80 (m, 4H), 2.90 (t, 1H), 3.00-3.25 (m, 4H), 3.88 (s, 3H), 4.35 (t, 1H), 6.85-7.18 (m, 6H), 7.20-7.28 (m, 1H), 7.43 (dt, 1H).

MS: [M+H]⁺=496.6.

### EXAMPLE 4

### N-{1-Cyclohexyl-2-(2-fluorophenyl)-4-[4-(2-methoxyphenyl)piperazin-1-yl]butyl}-O-methylhydroxylamine

### (RS,SR)-N-{1-Cyclohexyl-2-(2-fluorophenyl)-4-[4-(2-methoxyphenyl)-piperazin-1-yl]butyl}-O-methylhydroxylamine

### Example 4a

### (RS,RS)-N-{1-Cyclohexyl-2-(2-fluorophenyl)-4-[4-(2-methoxyphenyl)-piperazin-1-yl]butyl}-O-methylhydroxylamine

### Example 4b

To a solution of 0.098 g of the Compound of Example 2 and 1.12 mL of 3M HCl in 2.5 mL of EtOH, cooled at −5° C., was added (dropwise) 105 μL of 8 M BH₃-Py complex. The reaction mixture was allowed to warm to r.t. and stirred for 24 h. Afterwards, it was heated at 60° C. for 1.5 h. After cooling at 0° C., to the mixture was added 5 mL of water, 7.5 mL of EtOAc, and 2N NaOH up to pH 8. The organic layer was separated, washed with water (2×5 mL), dried (Na₂SO₄) and evaporated to dryness in vacuo affording 0.096 g of crude product, which was purified by flash chromatography (petroleum ether-EtOAc=70:30) to yield 0.037 g (37.5%) of the title compound as a diastereomeric mixture (RS,SR-RS,RS 8:2).

¹H-NMR (CDCl₃, δ): 1.10-1.90 (m, 11H), 1.92-2.50 (m, 4H), 2.50-2.75 (m, 4H), 2.92-2.98 (m, 1H), 2.98-3.20 (m, 4H), 3.25-3.38 (m, 4H), 3.55-3.70 (br, 1H), 3.88 (s, 3H), 6.85-7.30 (m, 7H), 7.35-7.48 (m, 1H).

MS: [M+H]⁺=470.6.

### (RS,SR)-N-{1-Cyclohexyl-2-(2-fluorophenyl)-4-[4-(2-methoxyphenyl)piperazin-1-yl]butyl}-O-methylhydroxylamine

### Example 4a

The Compound of Example 4 was further purified by flash chromatography (petroleum ether-EtOAc-1.6 N methanolic NH₃ gradient from 9:1:0.2 to a 8:2:0.2 to yield as the firstly eluted diastereomer the title Compound (61%) and a second group of fractions containing as a major product the opposite diastereomer.

MS: [M+H]⁺=470.39

### (RS,RS)-N-{1-Cyclohexyl-2-(2-fluorophenyl)-4-[4-(2-methoxyphenyl)piperazin-1-yl]butyl}-O-methylhydroxylamine

### Example 4b

The second group of fractions coming from Example 4a underwent further purification by flash chromatography (petroleum ether-EtOAc-1.6 N methanolic NH₃ 8:2:0.2 to yield the title compound (2.6%).

MS: [M+H]⁺=470.39

### EXAMPLE 5

### (RS,SR)- and (RS, RS)-1-Cyclohexyl-2-(2-fluorophenyl)-4-[4-(2-methoxyphenyl)piperazin-1-yl]butylamine

### (RS,SR)-1-Cyclohexyl-2-(2-fluorophenyl)-4-[4-(2-methoxyphenyl)-piperazin-1-yl]butylamine

### Upper TLC R_{f} Diastereomer

### Example 5a

### (RS,RS)-1-Cyclohexyl-2-(2-fluorophenyl)-4-[4-(2-methoxyphenyl)-piperazin-1-yl]butylamine

### Lower TLC R_{f} Diastereomer

### Example 5b

### (5a):(5b)=8:2

To a solution of 1.2 g of the Compound 1a and 0.735 g of NH₄Cl in 30 mL of MeOH at 0° C. was added 6.86 mL of 2M NH₃ in MeOH and 0.26 g of NaBH₃CN. The reaction was allowed to warm to at r.t. and the mixture was stirred for 45 days. The solvent was evaporated and the crude reaction mixture was taken up with H₂O (15 mL), cooled at 0° C., acidified with 3N HCl to a pH of 1. Afterwards, the mixture was basified with 35% NaOH up to pH 10 and extracted with CH₂Cl₂ (2×15 mL). The organic layer was separated, washed with brine (2×10 mL), dried (Na₂SO₄) and evaporated to dryness in vacuo to give 1.25 g of a crude product, which was purified by flash chromatography (CH₂Cl₂-1.6 N NH₃ in MeOH gradient from 97.5:2.5 to 95:5) affording compound 5a (upper R_{f}) (0.70 g, 58.1%) and the corresponding compound with lower R_{f} 5b (0.174, 14.4%).

MS: [M+H]⁺=440.6.

¹H-NMR 5a (CDCl₃, δ): 1.05-1.93 (m, 14H), 1.93-2.08 (m, 1H), 2.08-2.20 (m, 1H), 2.23-2.40 (m, 1H), 2.45-2.73 (m, 4H), 2.73-2.82 (m, 1H), 3.00-3.25 (m, 5H), 3.86 (s, 3H), 6.87 (d, 1H), 6.88-7.08 (m, 4H), 7.13-7.32 (m, 3H).

¹H-NMR 5b (CDCl₃, δ): 0.85-1.83 (m, 13H), 1.83-2.00 (m, 1H), 2.10-2.25 (m, 2H), 2.26-2.40 (m, 1H), 2.50-2.85 (m, 5H), 3.00-3.25 (m, 5H), 3.88 (s, 3H), 6.87 (d, 1H), 6.89-7.08 (m, 4H), 7.12 (dd, 1H), 7.17-7.30 (m, 2H).

### Example 5a

### (1R,2S) or (1S,2R)-1-Cyclohexyl-2-(2-fluorophenyl)-4-[4-(2-methoxyphenyl)piperazin-1-yl]butylamine

This compound was obtained as a single enantiomer from repetitive injection of the racemic mixture of Example 5a on a Chiralpak AD column (4.6×250 mm, eluent mixture: n-hexane-EtOH-DEA=90:10:0.1% at a flow rate of 1 mL/min, UV detection at 254 nm, duration 20 min, on a Waters 2795 instrument fitted with a 2996 DAD detector) and manual collection of the corresponding peak.

MS: [M+H]⁺=440.6.

### Example 5b

### (1S,2R) or (1R,2S)-1-Cyclohexyl-2-(2-fluorophenyl)-4-[4-(2-methoxyphenyl)piperazin-1-yl]butylamine

This compound was obtained as a single enantiomer from repetitive injection of the racemic mixture of Example 5a on a Chiralpak AD column (4.6×250 mm, eluent mixture: n-hexane-EtOH-DEA=90:10:0.1% at a flow rate of 1 mL/min, UV detection at 254 nm, duration 20 min, on a Waters 2795 instrument fitted with a 2996 DAD detector) and manual collection of the corresponding peak.

MS: [M+H]⁺=440.6.

### EXAMPLE 6

### N-{1-Cyclohexyl-2-(2-fluorophenyl)-4-[4-(2-methoxyphenyl)-piperazin-1-yl]butyl}formamide

0.54 g of the Compound 1a was dissolved in 0.724 mL of 98% formic acid and 1 mL of 98% formamide and the reaction mixture was irradiated in a microwave oven (Personal Chemistry) at 60W and at 202° C. for 2 cycles of 20 minutes each. The crude reaction mixture was diluted with 20 mL of water, basified with 1N NaOH to a pH of 9 and extracted with EtOAc (3×10 mL). The organic layer was washed, dried (Na₂SO₄) and evaporated to dryness to give a crude product, which was purified by flash chromatography (petroleum ether-EtOAc-1.6 N NH₃ in MeOH=50:50:2) affording the title compound (0.245 g; 42.6%).

¹H-NMR (CDCl₃, δ): 1.10-2.10 (m, 13H), 2.12-2.40 (m, 2H), 2.45-2.80 (˜d broad, 4H), 2.83-3.30 (˜s broad, 4H), 3.45 (qv, 1H), 3.86 (s, 3H), 4.26 (q, 1H), 6.86-7.30 (m, 8H), 7.75 (d, 1H), 8.12 (s, 1H).

MS: [M+H]⁺=468.6.

### EXAMPLE 7

### N-{(RS,SR)-1-Cyclohexyl-2-(2-fluorophenyl)-4-[4-(2-methoxyphenyl)-piperazin-1-yl]butyl}acetamide

This compound was obtained following the procedure described for the Compound of Example 3, but using as a starting material Compound 5a instead of the Compound of Example 1. Purification by flash chromatography (EtOAc-CH₂Cl₂-1.6 N NH₃ in MeOH=gradient from 80:20:3 to 70:30:3) afforded 60.9% of the title product.

¹H-NMR (CDCl₃, δ): 0.85-1.97 (m, 15H), 1.98-2.10 (m, 1H), 2.18-2.28 (m, 1H), 2.29-2.39 (m, 1H), 2.50-2.70 (m, 4H), 3.00-3.20 (m, 4H), 3.35-3.45 (m, 1H), 3.88 (s, 3H), 4.13 (dd, 1H), 5.08 (d, 1H), 6.85-7.30 (m, 8H).

MS: [M+H]⁺=482.7.

### EXAMPLE 8

### N-Acetyl-N-{(RS,SR)-1-cyclohexyl-2-(2-fluorophenyl)-4-[4-(2-methoxyphenyl)piperazin-1-yl]butyl}acetamide

The title compound was isolated during the chromatographic purification of the Compound of Example 7 from the fractions eluted after those containing the compound of Example 7 (0.012 g; 10.1% as a pure sample after flash chromatography).

¹H-NMR (CDCl₃, δ): 0.80-1.88 (m, 1H), 1.90 (s, 3H), 1.95-2.35 (m, 7H), 2.43-2.68 (m, 4H), 2.90-3.20 (m, 4H), 3.63-3.78 (m, 1H), 3.88 (s, 3H), 4.18 (t, 1H), 6.84-7.25 (m, 8H).

MS: [M+H]⁺=524.7.

### EXAMPLE 9

### N-{(RS,RS)-1-Cyclohexyl-2-(2-fluorophenyl)-4-[4-(2-methoxyphenyl)-piperazin-1-yl]butyl}acetamide

The title compound was obtained following the procedure described for the Compound of Example 7, but using as a starting material 5b instead of 5a. Usual work-up procedure afforded 96.1% of the title compound as a white solid.

¹H-NMR (CDCl₃, δ): 0.80-1.78 (m, 11H), 1.80-1.93 (m, 1H), 1.95-2.05 (m, 1H), 2.09 (s, 3H), 2.10-2.17 (m, 1H), 2.20-2.33 (m, 1H), 2.45-2.70 (m, 4H), 2.90-3.20 (m, 5H), 3.88 (s, 3H), 4.23 (dd, 1H), 5.40 (br, 1H), 6.82-7.33 (m, 8H).

MS: [M+H]⁺=482.7.

### EXAMPLE 10

### (RS,SR)- and (RS,RS)-{1-Cyclohexyl-2-(2-fluorophenyl)-4-[4-(2-methoxyphenyl)piperazin-1-yl]butyl}methylamine

### (RS,SR)-{1-Cyclohexyl-2-(2-fluorophenyl)-4-[4-(2-methoxyphenyl)-piperazin-1-yl}butyl methylamine

### Example 10a

### (RS,RS)-{1-Cyclohexyl-2-(2-fluorophenyl)-4-[4-(2-methoxyphenyl)-piperazin-1-yl]butyl methylamine

### Example 10b

Method A: To a solution of 0.30 g of Compound 1a and 3.41 mL of 2M metanolic methylamine in 2.7 mL of MeOH at 0° C., was added a solution of 0.853 mL of 4M HCl in i-PrOH and 0.0643 g of NaBH₃CN. The reaction was allowed to warm to r.t. and the reaction mixture was stirred at r.t. for 38 days. The solvent was evaporated and the crude reaction mixture was taken up with H₂O (15 mL), cooled to 0° C., acidified with 3N HCl to a pH of 1. Afterwards, the mixture was basified with 35% NaOH to a pH of 10 and extracted with CH₂Cl₂ (2×5 mL). The organic layer was separated, washed with brine (2×5 mL), dried (Na₂SO₄) and evaporated to dryness in vacuo. The crude product (0.35 g) was purified by flash chromatography (EtOAc-1.6 N NH₃ in MeOH=gradient from 100:2 to 95:5) affording pure compound 10a (upper TLC R_{f}, 0.208 g, 67.3%) and the corresponding compound with lower TLC R_{f} 10b (0.018 g, 5.8%), which, according to ¹H-NMR and LC, is a mixture ((10b):(10a)=8:2).

Method B: To a solution of 0.81 g of the Compound of Example 6 in 37 mL of anhydrous THF, was added 0.52 mL of the borane-dimethyl sulfide complex 10 M in THF. After 8 h stirring at room temperature and overnight resting, the reaction mixture was heated at reflux for 1 h after the addition of 37% HCl and methanol. Afterwards, the mixture was washed with EtOAc and the aqueous layer basified with 35% NaOH to a pH of 10 and extracted with CH₂Cl₂ (3×15 mL). The organic layer was separated, washed with brine (2×5 mL), dried (Na₂SO₄) and evaporated to dryness in vacuo. The crude product (0.69 g) was purified by flash chromatography (EtOAc-petroleum ether-1.6 N NH₃ in MeOH=5:5:0.15) affording the compound 10 as a diastereomeric mixture (65%), which can be resolved as above affording the single diastereoisomers. The composition of this mixture is similar to that obtained with method A.

MS: [M+H]⁺=454.7.

¹H-NMR 10a (CDCl₃, δ): 1.10-1.94 (m, 12H), 1.95-2.05 (m, 2H), 2.06-2.18 (m, 1H), 2.21 (s, 3H), 2.23-2.32 (m, 1H), 2.38-2.45 (m, 1H), 2.50-2.70 (m, 4H), 3.00-3.22 (m, 5H), 3.86 (s, 3H), 6.83-7.25 (m, 7H), 7.43 (dt, 1H).

¹H-NMR 10b (CDCl₃, δ): 0.80-1.83 (m, 12H), 1.83-2.40 (m, 5H), 2.24 (s, 0.75H), 2.44 (s, 2.25H), 2.48-2.75 (m, 4H), 2.95-3.22 (m, 5H), 3.86 (s, 3H), 6.85-7.45 (m, 8H).

### EXAMPLE 11

### {(RS,SR)-1-Cyclohexyl-2-(2-fluorophenyl)-4-[4-(2-methoxyphenyl)-piperazin-1-yl]butyl}dimethylamine

To a solution of 0.061 g of 5a in 2 mL of CH₃CN were added 0.104 mL of 37% HCHO, 0.026 g of NaBH₃CN and 30 μL of glacial acetic acid. The reaction mixture was stirred at r.t. for 4.5 h. Afterwards, the solvent was evaporated in vacuo, the residue was taken up with H₂O (10 mL), basified with 35% NaOH to a pH of 10 and extracted with CH₂Cl₂ (2×5 mL). The organic layer was separated, washed with brine (2×5 mL), dried (Na₂SO₄) and evaporated to dryness. The crude product (0.064 g) was purified by flash chromatography (CH₂Cl₂-1.6 N NH₃ in MeOH=97.5:2.5) affording the title compound (0.038 g, 58.5%).

¹H-NMR (CDCl₃, δ): 1.10-1.40 (m, 5H), 1.60-1.88 (m, 7H), 1.92-2.05 (m, 1H), 2.05-2.15 (m, 1H), 2.19 (s, 6H), 2.23-2.36 (m, 1H), 2.45-2.67 (m, 4H), 2.68-2.74 (m, 1H), 2.95-3.20 (m, 4H), 3.33 (ddd, 1H), 3.86 (s, 3H), 6.83-7.30 (m, 8H).

MS: [M+H]⁺=468.7.

### EXAMPLE 12

### (RS,SR)-{1-Cyclohexyl-2-(2-fluorophenyl)-4-[4-(2-methoxyphenyl)-piperazin-1-yl]butyl}isopropylamine

A mixture of 0.047 g of 5a, 31.4 μL of acetone, 0.068 g of sodium triacetoxyborohydride, 24.5 μL of AcOH and 1.1 mL of CH₂Cl₂ was stirred at r.t. for 8 h. After overnight resting, the mixture was basified with 1N NaOH to a pH of 9 and extracted with CH₂Cl₂ (5 mL). The organic layer was separated, washed with brine (2×3 mL), dried (Na₂SO₄) and evaporated to dryness in vacuo to give a crude, which was purified by flash chromatography (CH₂Cl₂-1.6 N NH₃ in MeOH=100:3) affording the title compound (0.037 g; 71.8%).

¹H-NMR (CDCl₃, δ): 0.75 (d, 3H), 0.91 (d, 3H), 1.05-1.86 (m, 12H), 1.90-2.08 (m, 2H), 2.08-2.20 (m, 1H), 2.20-2.33 (m, 1H), 2.44 (septet, 1H), 2.50-2.75 (m, 5H), 2.95-3.23 (m, 5H), 3.86 (s, 3H), 6.87 (d, 1H), 6.90-7.04 (m, 4H), 7.08 (dd, 1H), 7.10-7.24 (m, 1H), 7.45 (dd, 1H).

MS: [M+H]⁺=482.7.

### EXAMPLE 13

### (RS,SR)- and (RS,RS)-N-Benzyl-{1-cyclohexyl-2-(2-fluorophenyl)-4-[4-(2-methoxyphenyl)piperazin-1-yl]butyl}amine

To a solution of 0.064 g of Compound 1a and 47.8 μL of benzylamine in 2 mL of CH₃OH, at 0° C., was added 0.117 ml of 1.5 M HCl in EtOH and 0.014 g of NaBH₃CN. The reaction mixture was irradiated in a microwave oven (Personal Chemistry:Smith Creator) at 100° C. for 60 minutes. Afterwards, to the solution was added 95 μL of benzylamine, 1 mL of MeOH, 39 μL of 1.5 M HCl in EtOH and 0.014 g of NaBH₃CN and the mixture was irradiated for two additional cycles at 130° C. for 150 minutes and at 120° C. for 150 minutes. After another addition of reagents (47.8 μL of benzylamine, 1 mL of MeOH, 39 μL of 1.5 M HCl/EtOH and 0.014 g of sodium cyanoborohydride) the reaction mixture was irradiated at 130° C. for 150 minutes. The solvent was evaporated and the crude reaction mixture was taken up with H₂O (10 mL), cooled at 0° C., acidified with 4N HCl up to pH 1. Afterwards, the mixture was basified with 1N NaOH to a pH of 10 and extracted with CH₂Cl₂ (2×5 mL). The organic layer was separated, washed with brine (2×5 mL), dried (Na₂SO₄) and evaporated to dryness in vacuo. The crude product (0.21 g) was purified by LC-MS preparative chromatography [Xterra MS-C18 (30×50 mm, 5 μm), gradient A/B (A: Buffer aq. NH₄HCO₃ 20 mM at pH 8, B: CH₃CN) from 40% A and 60% B to 100% B, flow rate 50 ml/min, on a Waters 2795 instrument fitted with a ZQ MS and a 2996 DAD detector] affording the title compound (0.011 g, 14.2%) as a mixture of diastereomers=88:12 as per LC-MS analysis.

¹H-NMR of the major diastereomer (CDCl₃, δ): 0.80-1.78 (m, 12H), 1.80-2.00 (m, 2H), 2.00-2.10 (m, 1H), 2.12-2.30 (m, 1H), 2.35-2.70 (m, 5H), 2.83-3.08 (m, 4H), 3.12 (q, 1H), 3.31, 3.56 (2×d, 1.8H), 3.54, 3.72 (2×d, 0.2H), 3.77 (s, 3H), 6.75-7.43 (m, 13H).

MS: [M+H]⁺=530.7.

### EXAMPLE 14

### {(RS,SR)-1-Cyclohexyl-2-(2-fluorophenyl)-4-[4-(2-methoxyphenyl)-piperazin-1-yl]butyl}furan-2-ylmethylamine

The title compound was synthesised following the procedure reported for the Compound of Example 12, but using furan-2-carbaldehyde instead of acetone. After the usual work-up procedure, the crude was purified by flash chromatography (CHCl₃-MeOH=gradient from 100:2 to 100:3) affording the title compound (0.031 g; 44.5%) as a thick yellow oil.

¹H-NMR (CDCl₃, δ): 1.10-1.85 (m, 12H), 1.85-2.04 (m, 2H), 2.05-2.20 (m, 1H), 2.20-2.33 (m, 1H), 2.40-2.70 (m, 5H), 2.95-3.18 (m, 4H), 3.20 (dd, 1H), 3.50 (dd 2H), 3.86 (s, 3H), 6.01 (d, 1H), 6.26 (dd, 1H), 6.87 (d, 1H), 6.90-6.97 (m, 2H), 6.97-7.06 (m, 2H), 7.10 (t, 1H), 7.19 (q, 1H), 7.29 (d, 1H), 7.44 (t, 1H).

MS: [M+H]⁺=520.7.

### EXAMPLE 15

### (RS,SR)-1-Cyclohexyl-2-(2-fluorophenyl)-4-[4-(2-methoxyphenyl)-piperazin-1-yl]butyl}-bis-furan-2-ylmethylamine

The title compound was obtained as a by-product isolated during the chromatographic purification of the Compound of Example 14 (0.014 g; 17.4% after flash chromatography).

¹H-NMR (CDCl₃, δ): 1.12-1.85 (m, 12H), 1.88-2.10 (m, 2H), 2.15-2.30 (m, 1H), 2.40-2.70 (m, 4H), 2.90-3.28 (m, 5H), 3.39, 3.64 (2×d, 4H), 3.46 (ddd, 1H), 3.85 (s, 3H), 6.01 (s, 2H), 6.26 (s, 2H), 6.85-7.09 (m, 6H), 7.19-7.30 (m, 4H).

MS: [M+H]⁺=600.8.

### EXAMPLE 16

### (RS,SR)- and (RS,RS)-N-{-1-Cyclohexyl-2-(2-fluorophenyl)-4-[4-(2-methoxyphenyl)piperazin-1-yl]butyl}-N-methylacetamide

This compound was obtained following the procedure described for the Compound of Example 3, but using as a starting material the Compound of Example 10 instead of the Compound of Example 1. Purification by flash chromatography (petroleum ether-EtOAc-1.6 N NH₃ in MeOH=60:40:5) afforded 85.6% of the title product as a 7:3 mixture of (RS,SR) and (RS, RS) dastereoisomers. Yellow oil.

¹H-NMR (CDCl₃, δ): 1.05-2.00 (m, 11H), 1.80 (s, 3H), 2.00-2.15 (m, 3H), 2.20-2.40 (m, 1H), 2.49, 2.54 (2×s, 3H), 2.55-2.75 (m, 4H), 2.90-3.25 (m, 4H), 3.52 (t, 1H), 3.86 (s, 3H), 3.91-3.97 (m, 0.3H), 4.96 (m, 0.7H), 6.87-7.28 (m, 8H).

MS: [M+H]⁺=496.7.

### EXAMPLE 17

### (RS,SR)-{1-Cyclohexyl-2-(2-fluorophenyl)-4-[4-(2-methoxyphenyl)-piperazin-1-yl]butyl}carbamic acid methyl ester

Into a solution of 0.275 g of Compound 5a and 96.1 μL of TEA in 15 mL of CH₂Cl₂, cooled to 10° C., was added 53.7 μL of methyl chloroformate. The reaction mixture was stirred at this temperature for 1 h, then it was allowed to warm up to r.t. and stirred at the same temperature for another 1 h. Afterwards, it was diluted with 30 mL of ice-water, the organic layer was separated and the aqueous layer was extracted with 2×mL of CH₂Cl₂. The combined extracts were washed with water (10 mL), dried (Na₂SO₄) and evaporated to dryness. Purification of the crude product by flash chromatography (petroleum ether-EtOAc-MeOH=70:30:3) afforded 0.181 g (58.1%) of the title product as a thick brownish oil.

¹H-NMR (CDCl₃, δ): 0.80-1.80 (m, 10H), 1.80-2.10 (m, 3H), 2.12-2.40 (m, 2H), 2.40-2.75 (m, 4H), 2.90-3.20 (m, 4H), 3.30-3.45 (m, 1H), 3.54 (s, 3H), 3.70-3.78 (m, 1H), 3.82 (s, 3H), 4.33 (d, 1H), 6.82 (d, 1H), 6.87-7.05 (m, 4H), 7.11 (t, 1H), 7.12-7.30 (m, 2H).

MS: [M+H]⁺=498.7.

### EXAMPLE 18

{(RS,SR)-1-Cyclohexyl-2-(2-fluorophenyl)-4-[4-(2-methoxyphenyl)-piperazin-1-yl]butyl}urea

To a solution of 0.070 g of 5a in 2 mL of anhydrous THF, stirred at 0-5° C. under a N₂ stream, was added 32.3 μL of trimethylsilyl isocyanate. The reaction mixture was stirred at r.t. for 16 h, then heated at 50° C. for 4 h. After another addition of 32.3 μL of trimethylsilyl isocyanate, stirring was continued for 48 h at r.t. The mixture was poured into ice-water (5 mL), alkalinised with 1N NaOH (1 mL) and extracted with EtOAc (3×5 mL). The organic layer was separated, washed with brine, dried (Na₂SO₄) and evaporated to dryness in vacuo to give a crude product, purified by flash chromatography (CHCl₃-MeOH=90:10) to afford 0.028 g (36.5%) of the title product as a white solid.

¹H-NMR (CDCl₃, δ): 0.85-1.82 (m, 10H), 1.84-2.00 (m, 2H), 2.00-2.12 (m, 1H), 2.18-2.40 (m, 2H), 2.45-2.78 (m, 4H), 2.90-3.20 (m, 4H), 3.32-3.45 (m, 1H), 3.30-4.00 (m, 4H), 4.05-4.70 (m, 3H), 6.86 (d, 1H), 6.90-7.08 (m, 4H), 7.14 (t, 1H), 7.24-7.30 (m, 2H).

MS: [M+H]⁺=483.6.

### EXAMPLE 19

### 1-{(RS,SR)-1-Cyclohexyl-2-(2-fluorophenyl)-4-[4-(2-methoxyphenyl)-piperazin-1-yl]butyl}-3-methylurea

To a solution of 0.052 g of 5a in 0.8 mL of anhydrous DMF cooled at 0° C. was added under a N₂ stream 0.005 g of 60% NaH oil dispersion. The reaction mixture was stirred at 0° C. for 0.5 h and at r.t. for 1.5 h; then 21 μL of methyl isocyanate was added at 0-5° C. via a syringe and the resulting mixture was stirred at the same temperature for 0.5 h, then at r.t. overnight. Afterwards, it was diluted with 10 mL of iced water and extracted with 3×5 mL of Et₂O. The combined extracts were washed with water (10 mL), dried (Na₂SO₄) and evaporated to dryness in vacuo, affording 0.042 g (71.7%) of the title product as a white solid.

¹H-NMR (CDCl₃, δ): 0.80-1.85 (m, 10H), 1.85-2.00 (m, 2H), 2.00-2.13 (m, 1H), 2.20-2.40 (m, 2H), 2.45-2.70 (m, 4H), 2.67 (s, 3H), 3.00-3.20 (m, 4H), 3.35-3.45 (m, 1H), 3.86 (s, 3H), 3.90-3.97 (m, 2H), 4.03 (broad, 1H), 6.87 (d, 1H), 6.94-7.08 (m, 4H), 7.13 (t, 1H), 7.22-7.38 (m, 2H).

MS: [M+H]⁺=497.7.

### EXAMPLE 20

### 1-{(RS,SR)-1-Cyclohexyl-2-(2-fluorophenyl)-4-[4-(2-methoxyphenyl)-piperazin-1-yl]butyl}morpholine-4-carboxamide

Into a solution of 0.048 g of 5a and 22.8 μL of TEA in 2.1 mL of CH₂Cl₂, cooled at −12° C., was added 19.1 μL of morpholine-4-carbonyl chloride. The reaction mixture was allowed to warm up to r.t. and stirred for 24 h. Afterwards, it was heated at reflux for 6 h. After cooling to r.t., to the mixture was added 5 mL of water, 10 mL of CH₂Cl₂ and 3N NaOH to a pH of 10. The organic layer was separated, washed with water (2×5 mL), dried (Na₂SO₄) and evaporated to dryness in vacuo. Purification of the crude product by flash chromatography (CH₂Cl₂-MeOH=100:3), yielded 0.041 g (68.1%) of the title compound as a thick yellow oil.

¹H-NMR (CDCl₃, δ): 0.85-1.83 (m, 10H), 1.85-1.98 (m, 2H), 1.98-2.10 (m, 1H), 2.16-2.26 (m, 1H), 2.26-2.38 (m, 1H), 2.45-2.75 (m, 4H), 2.95-3.44 (m, 8H), 3.34-3.44 (m, 1H), 3.53-3.63 (m, 4H), 3.86 (s, 3H), 4.02-4.10 (m, 1H), 4.13 (d, 1H), 6.87 (d, 1H), 6.92-7.08 (m, 4H), 7.13 (t, 1H), 7.18-7.35 (m, 2H).

MS: [M+H]⁺=553.7.

### EXAMPLE 21

### 3-{(RS,SR)-1-Cyclohexyl-2-(2-fluorophenyl)-4-[4-(2-methoxyphenyl)-piperazin-1-yl]butyl}-1,1-dimethylurea

The title compound was prepared from the Compound of Example 5a using the method described for the Compound of Example 20, but using N,N-dimethylcarbamoyl chloride instead of morpholine-4-carbonyl chloride. Usual work-up procedure afforded 93.0% of the title compound as a thick oil.

¹H-NMR (CDCl₃, δ): 0.85-1.38 (m, 6H), 1.60-1.82 (m, 4H), 1.83-2.10 (m, 3H), 2.15-2.45 (m, 2H), 2.45-2.75 (m, 4H), 2.79, 2.82 (2s, 6H), 2.95-3.25 (m, 4H), 3.38-3.46 (m, 1H), 3.86 (s, 3H), 3.96-4.10 (m, 2H), 6.85 (d, 1H), 6.88-7.08 (m, 4H), 7.13 (t, 1H), 7.17-7.45 (m, 2H).

MS: [M+H]⁺=511.7.

### EXAMPLE 22

### 1-{(RS,SR)-1-Cyclohexyl-2-(2-fluorophenyl)-4-[4-(2-methoxyphenyl)-piperazin-1-yl]butyl}-1,3,3-trimethylurea

To a solution of 0.023 g of triphosgene in 2 ml of CH₂Cl₂ under a N₂ stream, a solution of 0.092 g of the Compound of Example 10 and 39 μL of DIPEA in 3 mL of CH₂Cl₂ was added dropwise over 1 hr. at r.t. The reaction mixture was stirred at the same temperature for 1 h. Afterwards, to the solution was added 39 μL of DIPEA and 0.445 mL of a 0.456 M solution of dimethylamine in toluene. After stirring overnight, to the mixture was added 5 mL of water, 5 mL of CH₂Cl₂ and 2N NaOH to a pH of 10. The organic layer was separated, washed with brine (2×5 mL), dried (Na₂SO₄) and evaporated to dryness. The oily crude product (0.099 g) was purified by flash chromatography (CHCl₃-MeOH=100:3) affording the title product (0.064 g, 60.1%).

¹H-NMR (CDCl₃, δ): 1.10-1.95 (m, 1H), 1.98-2.12 (m, 3H), 2.23-2.33 (m, 1H), 2.34 (s, 6H), 2.40-2.70 (m, 4H), 2.55 (s, 3H), 2.95-3.20 (m, 4H), 3.52 (ddd, 1H), 3.86 (s, 3H), 4.38 (m, 1H), 6.85-7.38 (m, 8H).

MS: [M+H]⁺=525.7.

### EXAMPLE 23

### 1-{(RS,SR)-1-Cyclohexyl-2-(2-fluorophenyl)-4-[4-(2-methoxyphenyl)-piperazin-1-yl]butyl}-3-methylthiourea

To a solution of 0.047 g of 5a in 0.65 mL of anhydrous DMF stirred at r.t. under N₂, was added 0.0078 g of methyl isothiocyanate and the resulting mixture was stirred for 3 h. Afterwards, it was diluted with 10 mL of iced water and extracted with 3×5 mL of Et₂O. The combined extracts were washed with water (10 mL), dried (Na₂SO₄) and evaporated to dryness in vacuo affording 0.051 g (93.0%) of the title product as a white solid.

¹H-NMR (CDCl₃, δ): 1.00-1.85 (m, 10H), 1.85-2.23 (m, 3H), 2.25-2.48 (m, 2H), 2.48-2.73 (m, 4H), 2.75-3.00 (bs, 3H), 3.00-3.20 (m, 4H), 3.40-3.60 (m, 1H), 3.87 (s, 3H), 5.30 (bs, 1H), 5.85 (bs, 1H), 6.86-7.32 (m, 8H).

MS: [M+H]⁺=513.7.

### EXAMPLE 24

### 3-{(RS,SR)-1-Cyclohexyl-2-(2-fluorophenyl)-4-[4-(2-methoxyphenyl)-piperazin-1-yl]butyl}-1,1-dimethylthiourea

To a solution of 0.205 g of 5a in 5 ml of toluene, at r.t. under N₂, was added 0.167 g of 1,1′-thiocarbonyldiimidazole and the reaction mixture was irradiated in a microwave oven (Personal Chemistry-Smith Creator) at 105° C. for 30 minutes. Afterwards, 1.23 mL of a 0.46 M solution of dimethylamine in toluene was added in several aliquots at r.t. The mixture was stirred for 5 h, then poured into water (10 mL) and extracted with EtOAc (3×5 mL). The organic layer was separated, washed with brine, dried (Na₂SO₄) and evaporated to dryness in vacuo to give a crude product which, purified by LC-MS preparative chromatography [Xterra MS-C18 (30×150 mm, 5 μm) column, gradient A/B (A: Buffer aq. NH₄HCO₃ 20 mM at pH 8, B: CH₃CN) from 80% A and 20% B to 100% B, flow rate 35 ml/min, on a Waters 2795 instrument fitted with a ZQ MS and a 2996 DAD detector], afforded 0.145 g (58.9%) of the title product.

¹H-NMR (CDCl₃, δ): 1.05-1.98 (m, 11H), 2.00-2.10 (m, 1H), 2.10-2.23 (m, 1H), 2.25-2.45 (m, 2H), 2.46-2.79 (m, 4H), 2.90-3.18 (m, 4H), 3.18 (s, 6H), 3.50-3.60 (m, 1H), 3.87 (s, 3H), 4.95-5.05 (m, 1H), 5.18 (d, 1H), 6.86-7.35 (m, 8H).

MS: [M+H]⁺=527.8.

### 3-{(1R,2S) or (1S,2R)-1-Cyclohexyl-2-(2-fluorophenyl)-4-[4-(2-methoxyphenyl)piperazin-1-yl]butyl}-1,1-dimethylthiourea

### Example 24A

This compound was obtained as a single enantiomer from repetitive injection of the racemic mixture of the compound of Example 24 on a Chiralpak AD column (4.6×250 mm, eluent mixture: n-hexane-EtOH-DEA=90:10:0.1% at a flow rate of 1 mL/min, UV detection at 254 nm, duration 20 min, on a Waters 2795 instrument fitted with a 2996 DAD detector) and manual collection of the corresponding peak.

MS: [M+H]⁺=527.8

### 3-{(1S,2R) or (1R,2S)-1-Cyclohexyl-2-(2-fluorophenyl)-4-[4-(2-methoxyphenyl)piperazin-1-yl]butyl}-1,1-dimethylthiourea

### Example 24B

This compound was obtained as a single enantiomer from repetitive injection of the racemic mixture of the compound of Example 24 on a Chiralpak AD column (4.6×250 mm, eluent mixture: n-hexane-EtOH-DEA=90:10:0.1% at a flow rate of 1 mL/min, UV detection at 254 nm, duration 20 min, on a Waters 2795 instrument fitted with a 2996 DAD detector) and manual collection of the corresponding peak.

MS: [M+H]⁺=527.8

### EXAMPLE 25

### 2-{1-Cyclohexyl-[(E)-hydroxyiminomethyl]-3-[4-(2,3-dihydrobenzo[1,4]dioxin-5-yl)piperazin-1-yl]Propyl}benzonitrile

### 2-{1-Cyclohexanecarbonyl-3-[4-(2,3-dihydrobenzo[1,4]dioxin-5-yl)-piperazin-1-yl]propyl}benzonitrile (Compound 25a)

This compound was synthesised as previously described in WO 03/106443 A1 (Example 13).

### 2-{1-Cyclohexyl-[(E)-hydroxyiminomethyl]-3-[4-(2,3-dihydrobenzo[1,4]dioxin-5-yl)piperazin-1-yl]propyl}benzonitrile

The title compound was prepared using the method described for Compound of Example 1 but using as a starting material Compound 25a instead of the Compound 1a. Purification by flash chromatography (CH₂Cl₂-1.6 N NH₃ in MeOH gradient from 97:3 to 95:5) afforded 48.8% of the title product.

¹H-NMR (CDCl₃, δ): 1.30-2.03 (m, 10H), 2.60-2.80 (m, 2H), 2.80-2.90 (m, 4H), 2.99 (q, 2H), 3.10-3.50 (m, 6H), 4.30-4.45 (m, 4H), 6.30 (bs, 1H), 6.55-6.70 (m, 2H), 6.82 (t, 1H), 7.52-7.65 (m, 2H), 7.70 (d, 1H), 8.00 (d, 1H).

MS: [M+H]⁺=489.6.

### EXAMPLE 26

### (R,S)-2-{1′-{Cyclohexyl-[(E)-methoxyimino]methyl}-3-[4-(2,3-dihydrobenzo[1,4]dioxin-5-yl)-piperazin-1-yl]propyl}benzonitrile

To a solution of 0.150 g of Compound 25a in 5 mL of EtOH—H₂O (4:1) was added 0.265 g of O-methylhydroxylamine hydrochloride and the resulting solution was irradiated in a microwave oven (Personal Chemistry, Smith Synthesiser) at 130° C. for 30 minutes. Afterwards, additional 0.137 g of O-methylhydroxylamine hydrochloride was added and the reaction was irradiated at the same temperature for 40 minutes. The mixture was poured into 10 ml of 10% aq. NaHCO₃ and extracted with CH₂Cl₂ (3×15 mL). The organic layer was washed, dried (Na₂SO₄) and evaporated to dryness to give a crude product, which was purified by flash chromatography (petroleum ether-acetone 85:15) affording the title compound (0.040 g; 25.1%).

¹H-NMR (CDCl₃, δ): 1.05-1.80 (m, 10H), 1.80-1.95 (m, 1H), 2.18-2.30 (m, 1H), 2.35-2.50 (m, 2H), 2.50-2.75 (m, 4H), 2.95-3.20 (m, 5H), 3.95 (s, 3H), 4.20-4.38 (m, 5H), 6.54-6.60 (m, 2H), 6.79 (t, 1H), 7.32 (t, 1H), 7.55 (t, 1H), 7.65 (dd, 2H).

MS: [M+H]⁺=503.7.

### EXAMPLE 27

### E(Z)-5-[4-(2,3-Dihydrobenzo[1,4]dioxin-5-yl)piperazin-1-yl]-3-(2-fluorophenyl)pentan-2-one oxime

### 5,5-Diethoxy-3-(2-fluorophenyl)pentan-2-one (Compound 27a)

To a suspension of 0.787 g of 60% NaH oil dispersion in 15 mL of anhydrous DMF was added dropwise via a syringe under a N₂ stream a solution of 2.50 g of 2-fluorophenylacetone in 15 mL of anhydrous DMF. The reaction mixture was stirred at r.t. for 1.5 h, then 3.70 mL of 2-bromoacetaldehyde diethyl acetal was added and the resulting mixture was stirred at r.t. overnight. Afterwards, the reaction mixture was poured into water (200 mL) and extracted with 3×80 mL of EtOAc. The organic layer was washed, dried (Na₂SO₄) and evaporated to dryness in vacuo affording a crude product, which was purified by flash chromatography (petroleum ether-EtOAc=100:5) affording 4.14 g (94.0%) of the title product as a yellowish oil.

¹H-NMR (CDCl₃, δ): 1.12-1.25 (m, 6H), 1.90-2.00 (m, 1H), 2.12 (s, 3H), 2.45-2.55 (m, 1H), 3.38-3.50 (m, 2H), 3.52-3.60 (m, 1H), 3.60-3.70 (m, 1H), 4.20 (t, 1H), 4.36 (t, 1H), 7.08-7.30 (m, 4H).

MS: [M+H]⁺=269.3.

### 3-(2-Fluorophenyl)-4-oxopentanal (Compound 27b)

A mixture of 1.00 g of Compound 27a, 30 mL of 50% aq. trifluoroacetic acid and 60 mL of CH₂Cl₂ was stirred for 2 h at r.t. The organic layer was separated, washed with brine (2×20 mL), dried (Na₂SO₄) and evaporated to dryness in vacuo to afford a reddish oil (0.72 g, 99.4%), that was kept under N₂ and used in the next step without further purification.

¹H-NMR (CDCl₃, δ): 2.16 (s, 3H), 2.65 (dd, 1H), 3.47 (dd, 1H), 4.58 (dd, 1H), 7.12-7.18 (m, 3H), 7.25-7.35 (m, 1H), 9.80 (s, 1H).

MS: [M+H]⁺=195.2.

### 5-[4-(2,3-Dihydrobenzo[1,4]dioxin-5-yl)piperazin-1-yl]-3-(2-fluorophenyl)pentan-2-one (Compound 27c)

The title compound was synthesised following the procedure reported for the Compound of Example 12, but using 1-(2,3-dihydrobenzo[1,4]dioxin-5-yl)piperazine instead of 5a and the Compound 27b instead of acetone. After the usual work-up procedure, the crude product was purified by flash chromatography (petroleum ether-EtOAc=1:1) affording the title compound as a colourless oil (0.21 g; 78.7%).

¹H-NMR (CDCl₃, δ): 1.75-1.88 (m, 1H), 2.14 (s, 3H), 2.30-2.50 (m, 3H), 2.50-2.75 (m, 4H), 2.98-3.18 (m, 4H), 4.15 (t, 1H), 4.24-4.37 (m, 4H), 6.55 (dd, 1H), 6.61 (dd, 1H), 6.78 (t, 1H), 7.05-7.17 (m, 2H), 7.20-7.30 (m, 2H).

MS: [M+H]⁺=399.5.

### E(Z)-5-[4-(2,3-Dihydrobenzo[1,4]dioxin-5-yl)piperazin-1-yl]-3-(2-fluorophenyl)pentan-2-one oxime

The title compound was prepared using the method described for the Compound of Example 1 but using as a starting material Compound 27c instead of Compound 1a. After 5 hours stirring, usual work-up and purification by flash chromatography (EtOAc-1.6 N NH₃ in MeOH=98:2) afforded the title compound as a white solid (0.124 g; 79.8%) as a single geometrical isomer.

¹H-NMR (CDCl₃, δ): 1.78 (s, 3H), 1.98-2.10 (m, 1H), 2.28-2.40 (m, 2H), 2.40-2.53 (m, 1H), 2.53-2.85 (m, 4H), 3.00-3.30 (m, 4H), 3.93 (t, 1H), 4.20-4.40 (m, 4H), 6.55 (dd, 1H), 6.61 (dd, 1H), 6.79 (t, 1H), 7.04-7.18 (m, 2H), 7.20-7.30 (m, 2H), 8.11 (bs, 1H).

MS: [M+H]⁺=414.5.

### EXAMPLE 28

### 5-[4-(2,3-Dihydrobenzo[1,4]dioxin-5-yl)piperazin-1-yl]-3-(2-fluorophenyl)-pentan-2-one O-methyloxime

The title compound was synthesised following the procedure reported for the Compound of Example 27, but using O-methylhydroxylamine.HCl instead of hydroxylamine hydrochloride. After the usual work-up procedure, the crude product was purified by flash chromatography (CH₂Cl₂-MeOH=95:5) affording the title compound (colourless oil, 82.4%) as a mixture of geometrical isomers (Z)/(E) or (E)/(Z)=9/1.

¹H-NMR (CDCl₃, δ): 1.63 (s, 2.7H), 1.67 (s, 0.3H), 1.80-1.90 (m, 1H), 2.15-2.40 (m, 3H), 2.40-2.75 (m, 4H), 2.90-3.20 (m, 4H), 3.73 (s, 0.3H), 3.81 (s, 2.7H), 3.82-3.85 (m, 1H), 4.12-4.30 (m, 4H), 6.53 (dd, 1H), 6.56 (dd, 1H), 6.69 (t, 1H), 6.96 (t, 1H), 7.03 (t, 1H), 7.10-7.30 (m, 2H).

MS: [M+H]⁺=428.5.

### EXAMPLE 29

### (RS,SR)- and (RS,RS)-4-[4-(2,3-Dihydrobenzo[1,4]dioxin-5-yl)-piperazin-1-yl]-2-(2-fluorophenyl)-1-methylbutylamine

To a stirred solution of 0.10 g of the Compound of Example 27 and 0.115 g of nickel (II) chloride hexahydrate in 10 mL of MeOH, cooled at −78° C., was added portionwise 0.0916 g of sodium borohydride (the reaction was strongly exotermic). After spontaneous heating to r.t. during 2 hours, the solvent was evaporated and the crude reaction mixture was taken up with H₂O (5 mL), basified with 1N NaOH and extracted with CH₂Cl₂ (3×5 mL). The organic layer was washed, dried (Na₂SO₄) and evaporated to dryness to give a crude product, which was purified by flash chromatography (CHCl₃-1.6 N NH₃ in MeOH=98:2) affording the title compound (0.04 g; 41.4%) as a mixture of diastereomers (RS,SR)— (RS, RS) 7:3.

¹H-NMR (CDCl₃, δ): 0.94 (d, 1H), 1.12 (d, 2H), 1.40 (bs, 2H), 1.80-1.92 (m, 1H), 1.93-2.05 (m, 1H), 2.05-2.20 (m, 1H), 2.20-2.32 (m, 1H), 2.40-2.75 (m, 4H), 2.80-2.90 (m, 1H), 2.90-3.10 (m, 4H), 3.10-3.20 (m, 1H), 4.15-4.35 (m, 4H), 6.50 (dd, 1H), 6.55 (dd, 1H), 6.74 (t, 1H), 6.96-7.07 (m, 1H), 7.09 (t, 1H), 7.18 (t, 1H), 7.20-7.28 (m, 1H).

MS: [M+H]⁺=400.5.

### EXAMPLE 30

### (RS,SR) and (RS,RS)-[4-[4-(2,3-Dihydrobenzo[1,4]dioxin-5-yl)-piperazin-1-yl]-2-(2-fluorophenyl)-1-methylbutyl]methylamine

This compound was obtained following the procedure described for the Compounds of Example 10, but using as a starting material Compound 27c instead of Compound 1a and stirring the reaction mixture at r.t. for 2 days instead of 38 days. Purification by flash chromatography (CHCl₃-1.6 N NH₃ in MeOH=95:5) afforded 55.9% of the title product as a 85:15 mixture of the (RS,SR) and (RS,RS) diastereomers.

¹H-NMR (CDCl₃, δ): 0.95 (d, 0.45H), 1.10 (d, 2.55H), 1.55 (bs, 1H), 1.83-1.98 (m, 1H), 2.00-2.16 (m, 1H), 2.16-2.23 (m, 1H), 2.23-2.33 (m, 1H), 2.38 (s, 2.55H), 2.48 (s, 0.45H), 2.50-2.70 (m, 4H), 2.75-2.88 (m, 1H), 2.90-3.15 (m, 5H), 4.22-4.38 (m, 4H), 6.58 (dd, 1H), 6.62 (dd, 1H), 6.78 (t, 1H), 7.03 (t, 1H), 7.13 (t, 1H), 7.18-7.32 (m, 2H).

MS: [M+H]⁺=414.5.

### EXAMPLE 31

### E(Z)-3-(2-Fluorophenyl)-5-{4-[2-(2,2,2-trifluoroethoxy)phenyl]-piperazin-1-yl}pentan-2-one oxime

### 1-[2-(2,2,2-Trifluoroethoxy)phenyl]piperazine (Compound 31a)

A mixture of 10.6 g of 2-(2,2,2-trifluoroethoxy)aniline and 10.10 g of bis-(2-chloroethyl)amine hydrochloride in 30 mL of 1,2-dichlorobenzene and 3 mL of n-hexanol was heated at 185° C. for 5 h. After cooling to r.t., at the mixture were added 100 mL of 2 M NaOH and the aqueous layer was extracted with 2×100 mL of Et₂O. The combined organic layers were washed, dried (Na₂SO₄) and evaporated to dryness to give a crude product, which was purified by flash chromatography (CHCl₃-1.6 N NH₃ in MeOH=gradient from 100:1 to 100:5) affording the title compound as a dark yellow oil (12.60 g; 87.3%).

¹H-NMR (CDCl₃, δ): 2.70 (bs, 1H), 3.08 (s, 8H), 4.40 (q, 2H), 6.82-7.13 (m, 4H).

MS: [M+H]⁺=261.3.

### 3-(2-Fluorophenyl)-5-{4-[2-(2,2,2-trifluoroethoxy)phenyl]piperazin-1-yl}pentan-2-one (Compound 31b)

The title compound was synthesised following the procedure reported for the Compound of Example 12, but using Compound 31a instead of 5a and Compound 27b instead of acetone. After the usual work-up procedure, the crude product was purified by flash chromatography (petroleum ether-EtOAc=1:1) affording the title compound (61.5%) as a colourless oil.

¹H-NMR (CDCl₃, δ): 1.75-1.92 (m, 1H), 2.14 (s, 3H), 2.30-2.50 (m, 3H), 2.50-2.75 (m, 4H), 2.98-3.20 (m, 4H), 4.15 (t, 1H), 4.41 (q, 2H), 6.89-7.03 (m, 3H), 7.03-7.18 (m, 3H), 7.20-7.35 (m, 2H).

MS: [M+H]⁺=439.5.

### E(Z)-3-(2-Fluorophenyl)-5-{4-[2-(2,2,2-trifluoroethoxy)phenyl]piperazin-1-yl}pentan-2-one oxime

The title compound was prepared using the method described for the Compound of Example 1 but using, as a starting material, Compound 31b instead of the Compound 1a. After 6 h at reflux, usual work-up and purification by flash chromatography (CH₂Cl₂-MeOH=95:5) afforded 92.8% of the title product as a white solid with undetermined but clearly predominant stereochemistry (E or Z>95%).

¹H-NMR (CDCl₃, δ): 1.78 (s, 3H), 2.03-2.18 (m, 1H), 2.30-2.50 (m, 3H), 2.50-2.83 (m, 4H), 3.00-3.25 (m, 4H), 3.93 (t, 1H), 4.39 (q, 2H), 6.94-7.20 (m, 6H), 7.20-7.35 (m, 2H), 8.85 (bs, 1H).

MS: [M+H]⁺=454.5.

### EXAMPLE 32

### E(Z)-3-(2-Fluorophenyl)-5-{4-[2-(2,2,2-trifluoroethoxy)phenyl]-piperazin-1-yl}pentan-2-one O-methyloxime

The title compound was synthesised following the procedure reported for the Compound of Example 28, but using as starting material Compound 31b instead of Compound 27c. After the usual work-up procedure, the crude product was purified by flash chromatography (CH₂Cl₂-MeOH=95:5) affording the title compound as a colourless oil (93.8%) with undetermined but clearly predominant stereochemistry (E or Z>95%).

¹H-NMR (CDCl₃, δ): 1.73 (s, 3H), 1.90-2.03 (m, 1H), 2.28-2.50 (m, 3H), 2.50-2.73 (m, 4H), 3.00-3.25 (m, 4H), 3. 88 (s, 3H), 3.90-3.97 (m, 1H), 4.41 (q, 2H), 6.92-7.20 (m, 6H), 7.20-7.35 (m, 2H).

MS: [M+H]⁺=468.5.

### EXAMPLE 33

### (RS,SR)- and (RS,RS)-2-(2-Fluorophenyl)-1-methyl-4-{4-[2-(2,2,2-trifluoroethoxy)phenyl]piperazin-1-yl}butylamine (33a):(33b)=65:35

### (RS,SR)-2-(2-Fluorophenyl)-1-methyl-4-{4-[2-(2,2,2-trifluoroethoxy)phenyl]-piperazin-1-yl}butylamine: upper TLC R_{f} diastereomer

### Example 33a

### (RS,RS)-2-(2-Fluorophenyl)-1-methyl-4-{4-[2-(2,2,2-trifluoroethoxy)-phenyl]piperazin-1-yl }butylamine: lower TLC R_{f} diastereomer

### Example 33b

These compounds were prepared according to the procedure described for the Compound of Example 29, but using as starting material the Compound of Example 31 instead of the Compound of Example 27. The reaction was carried out at −20° C. instead of −78° C. and another equivalent of reagents was added to complete the transformation. The crude product was purified by flash chromatography (CHCl₃-1.6 N NH₃ in MeOH=98:2) affording the title compound (50%) as a 65:35 diastereomers mixture Afterwards, the pair of diastereomers was separated by preparative LC-MS chromatography [Xterra MS-C18 (19×100 mm, 5 μm) column; eluent mixture: buffer aq. NH₄HCO₃ 20 mM at pH 8/CH₃CN=65:35, flow rate 20 ml/min, on a ZQ MS platform instrument fitted with a 2996 DAD detector] affording 25.8% of 33a (the upper R_{f} diastereomer) and 14.1% of 33b (the lower R_{f} diastereomer).

MS: [M+H]⁺=440.5.

¹H-NMR 33a (CDCl₃, δ): 1.16 (d, 3H), 1.83-1.98 (m, 1H), 1.98-2.09 (m, 1H), 2.10-2.23 (m, 1H), 2.24-2.36 (m, 1H), 2.40-2.70 (m, 4H), 2.85-2.95 (m, 1H), 3.00-3.12 m, 4H), 3.12-3.22 (m, 1H), 4.39 (q, 2H), 6.88-6.94 (d, 1H), 6.94-7.02 (t, 2H), 7.06-7.10 (dt, 2H), 7.12-7.17 (t, 1H), 7.18-7.34 (m, 2H).

¹H-NMR 33b (CDCl₃, δ): 0.97 (d, 3H), 1.70 (bs, 2H), 1.83-1.98 (m, 1H), 2.10-2.35 (m, 3H), 2.40-2.70 (m, 4H), 2.85-2.95 (m, 1H), 2.97-3.25 (m, 5H), 4.39 (q, 2H), 6.88-6.94 (d, 1H), 6.94-7.02 (t, 2H), 7.02-7.09 (m, 2H), 7.09-7.15 (t, 1H), 7.17-7.26 (m, 2H).

### EXAMPLE 34

### 5-{3-[4-(2,3-Dihydrobenzo[1,4]dioxin-5-yl)-piperazin-1-yl]-1-phenylpropyl }pyrrolidin-2-one

### 4-Nitro-3-phenylbutyric acid methyl ester (Compound 34a)

A mixture of 4.05 g of methyl cinnamate, 6.77 mL of nitromethane and 0.627 mL of N,N,N′,N′-tetramethylguanidine was stirred at r.t. for 48 h. Afterwards, it was diluted with 25 mL of diethyl ether and acidified with 12 mL of 1N HCl. The organic layer was separated, washed, dried (Na₂SO₄) and evaporated to dryness to give 4.84 g of a crude product, which was purified by flash chromatography (petroleum ether-EtOAc 9:1) affording the title compound (3.4 g; 61.0%) as a yellow oil.

¹H-NMR (CDCl₃, δ): 2.78 (d, 2H), 3.62 (s, 3H), 3.95 (quintet, 1H), 4.60-4.80 (m, 2H), 7.20-7.40 (m, 5H).

MS: [M+H]⁺=224.2.

### 4-Nitro-3-phenylheptanedioic acid dimethyl ester (Compound 34b)

Upper TLC Diastereomer

A mixture of 3.4 g of Compound 34a in 20 mL of tert-butanol, 1.35 mL of methyl acrylate and 0.237 mL of Triton B® was stirred at r.t. for 72 h. The reaction mixture was diluted with 25 mL of diethyl ether and acidified with 12 mL of 1N HCl. The organic layer was separated, washed, dried (Na₂SO₄) and evaporated to dryness to give 4.4 g of a crude product, which was purified by flash chromatography (petroleum ether-EtOAc=9:1) affording the title compound as a yellow oil (2.26 g; 48.8%) (upper R_{f} diastereomer) and the corresponding diastereomer with lower R_{f} (1.70 g, 36.7%).

¹H-NMR of the upper R_{f} diastereomer (CDCl₃, δ): 1.70-1.90 (m, 1H), 1.90-2.10 (m, 1H), 2.10-2.30 (m, 2H), 2.50-2.80 (m, 2H), 3.40 (s, 3H), 3.50 (s, 3H), 3.60-3.70 (m, 1H), 4.83 (ddd, 1H), 7.10-7.30 (m, 5H).

MS: [M+H]⁺=310.3.

### 3-(5-Oxopyrrolidin-2-yl)-3-phenylpropionic acid methyl ester (Compound 34c) and 3-(5-Oxo-3-phenylpyrrolidin-2-yl)propionic acid methyl ester

A suspension of 5.25 g of Compound 34b and a catalytic amount of Ni-Raney in 280 mL of toluene was hydrogenated at ambient pressure and r.t. for 16 h. The mixture was then filtered to remove the catalyst and the filtrate was concentrated in vacuo to give a crude oil which was purified by flash chromatography (CH₂Cl₂-MeOH=99:1) affording 1.62 g (38.6%) of a yellow oil characterized as a mixture of the title compound and the regioisomer 3-(5-oxo-3-phenylpyrrolidin-2-yl)propionic acid methyl ester. The mixture was used in the next step without further purification.

MS: [M+H]⁺=248.3.

### 3-(5-Oxopyrrolidin-2-yl)-3-phenylpropionic acid (Compound 34d) and 3-(5-oxo-3-phenylpyrrolidin-2-yl)propionic acid

A solution of 1.62 g. of the mixture of Compound 34c in 6.43 mL of 1N NaOH was heated at 60° C. for 4 h. After cooling to 0° C., at the mixture were added an excess of 1N HCl. The resulting precipitate was filtered and dried in vacuo affording a white solid (1.0 g, 66.0%) of the title compound and the corresponding regioisomer 3-(5-oxo-3-phenylpyrrolidin-2-yl)propionic acid. The mixture was used in the next step without further purification.

MS: [M+H]⁺=234.3.

### 5-{3-[4-(2,3-Dihydrobenzo[1,4]dioxin-5-yl)piperazin-1-yl]-3-oxo-1-phenylpropyl}pyrrolidin-2-one (Compound 34e)

To a solution of 0.90 g of the mixture of Compound 34d and the by-product 3-(5-oxo-3-phenylpyrrolidin-2-yl)propionic acid in 15 mL of CH₂Cl₂ were added 0.85 g of 1-(2,3-dihydrobenzo[1,4]dioxin-5-yl)piperazine, 0.89 g of 1-[3-(dimethylaminopropyl]-3-ethyl-carbodiimide hydrochloride and 1.08 mL of TEA. The reaction mixture was stirred at r.t. overnight. To the mixture were first added 20 mL of 0.05N HCl and then 20 mL of 0.05N NaOH. The organic layer was separated, washed with brine (2×7 mL), dried (Na₂SO₄) and evaporated to dryness. The crude oil was purified by flash chromatography (CH₂Cl₂-MeOH=98:2) affording the title product (0.213 g, 12.7%) together with the by-product 5-{3-[4-(2,3-dihydrobenzo[1,4]dioxin-5-yl)-piperazin-1-yl]-3-oxopropyl}-4-phenylpyrrolidin-2-one (0.739 g, 44.0%).

¹H-NMR (CDCl₃, δ): 1.30-1.55 (m, 2H), 2.20-2.40 (m, 2H), 2.65 (d, 2H), 2.85-3.00 (m, 4H), 3.40-3.47 (m, 2H), 3.60-3.85 (m, 2H), 3.77 (q, 1H), 3.93-4.05 (m, 1H), 4.16-4.33 (m, 4H), 6.44 (d, 1H), 6.60 (d, 1H), 6.75 (t, 1H), 7.18-7.36 (m, 5H), 7.60 (s, 1H).

MS: [M+H]⁺=436.6.

### 5-{3-[4-(2,3-Dihydrobenzo[1,4]dioxin-5-yl)piperazin-1-yl-1-phenylpropyl}pyrrolidin-2-one

To a solution of 0.20 g of Compound 34e and its regioisomer in 10 mL of anhydrous THF was added 0.035 g of LiAlH₄. The suspension was stirred at r.t. for 1 h and then was heated at reflux for 16 h. After cooling to 0° C., to the mixture were added 2 mL of THF and some drops of 0.5N NaOH. The solvent was evaporated in vacuo and the crude product was purified by flash chromatography (CH₂Cl₂-MeOH=95:5) to yield 0.117 g (60.3%) of the title compound.

¹H-NMR (CDCl₃, δ): 1.00-1.25 (m, 2H), 1.36-1.60 (m, 2H), 2.30 (t, 2H), 2.50-2.65 (m, 4H), 2.60-2.80 (m, 2H), 3.08 (m, 4H), 3.75 (q, 1H), 3.82-3.90 (m, 1H), 4.20-4.35 (m, 4H), 6.50-6.62 (m, 2H), 6.77 (t, 1H), 7.08 (bs, 1H), 7.18-7.38 (m, 5H).

MS: [M+H]⁺=422.5.

### EXAMPLE 35

### (RS,SR) or (RS,RS)-5-{3-[4-(2,3-Dihydrobenzo[1,4]dioxin-5-yl)piperazin-1-yl]-1-phenylpropyl}-1-ethylpyrrolidin-2-one

### 3-(1-Ethyl-5-oxopyrrolidin-2-yl)-3-phenylpropionic acid methyl ester (Compound 35a) and 3-(1-Ethyl-5-oxo-3-phenylpyrrolidin-2-yl)propionic acid methyl ester

The title compounds were obtained as the main by-products in the procedure described for Compound 34c when ethanol was used instead of toluene as a reaction solvent. Purification by flash chromatography (CH₂Cl₂-MeOH=99:1) afforded a mixture of product 35a and its regioisomer.

MS: [M+H]⁺=276.4.

### 3-(1-Ethyl-5-oxo-pyrrolidin-2-yl)-3-phenylpropionic acid (Compound 35b) and 3-(1-Ethyl-5-oxo-3-phenylpyrrolidin-2-yl)propionic acid

The title compound was synthesised following the procedure reported for the Compound 34d, but using as a starting material Compound 35a instead of Compound 34c. The obtained mixture was used without further purification.

MS: [M+H]⁺=262.3.

### 5-{3-[4-(2,3-Dihydrobenzo[1,4]dioxin-5-yl)piperazin-1-yl]-3-oxo-1-phenylpropyl}-1-ethylpyrrolidin-2-one (Compound 35c)

This compound was obtained following the procedure described for the Compound 34e, but using as a starting material the mixture of Compound 35b and its regiosomer instead of Compound 34d. Purification by flash chromatography (CH₂Cl₂-MeOH=98.5:1.5) afforded 14.5% of the title product.

¹H-NMR (CDCl₃, δ): 1.15 (t, 3H), 1.70-1.86 (m, 1H), 2.12-2.33 (m, 3H), 2.40 (dd, 1H), 2.76-2.98 (m, 5H), 2.98-3.13 (m, 2H), 3.30-3.48 (m, 2H), 3.60-3.70 (m, 2H), 3.70-3.85 (m, 2H), 4.10-4.33 (m, 4H), 6.42 (d, 1H), 6.60 (d, 1H), 6.75 (t, 1H), 7.18-7.36 (m, 5H).

MS: [M+H]⁺=464.6.

### (RS,SR) or (RS,RS)-5-{3-[4-(2,3-Dihydrobenzo[1,4]dioxin-5-yl)piperazin-1-yl]-1-phenylpropyl}-1-ethylpyrrolidin-2-one

The title compound was obtained following the procedure described for the Compound of Example 34, but using Compound 35c as starting material instead of Compound 34e. Purification by flash chromatography (CH₂Cl₂-MeOH=95.5:4.5) afforded 65.0% of the title product as almost pure diastereomer with unknown absolute stereochemistry.

¹H-NMR (CDCl₃, δ): 1.12 (t, 3H), 1.42-1.62 (m, 3H), 1.62-1.82 (m, 1H), 2.16-2.50 (m, 3H), 2.50-2.65 (m, 4H), 2.80-3.00 (m, 2H), 2.95-3.12 (m, 4H), 3.12-3.16 (m, 1H), 3.60-3.70 (m, 1H), 3.78 (sextet, 1H), 4.18-4.32 (m, 4H), 6.52 (dd, 1H), 6.60 (dd, 1H), 6.75 (t, 1H), 7.13-7.33 (m, 5H).

MS: [M+H]⁺=450.2.

### Example 35a

### 5-{3-[4-(2,3-Dihydrobenzo[1,4]dioxin-5-yl)piperazin-1-yl]-1-phenylpropyl }-1-ethylpyrrolidin-2-one—enantiomer 1

This compound was obtained as a single enantiomer from repetitive injection of the racemic mixture of Example 35 on a Chiralpak AD column (4.6×250 mm, eluent mixture: n-hexane-iPrOH-DEA=85:15:0.1% at a flow rate of 1 mL/min, UV detection at 254 nm, duration 20 min, on a Waters 2795 instrument fitted with a 2996 DAD detector) and manual collection of the corresponding peak.

MS: [M+H]⁺=450.2.

### Example 35b

### 5-{3-[4-(2,3-Dihydrobenzo[1,4]dioxin-5-yl)piperazin-1-yl]-1-phenylpropyl}-1-ethylpyrrolidin-2-one—enantiomer 2

This compound was obtained as a single enantiomer from repetitive injection of the racemic mixture of Example 35 on a Chiralpak AD column (4.6×250 mm, eluent mixture: n-hexane-iPrOH-DEA=85:15:0.1% at a flow rate of 1 mL/min, UV detection at 254 nm, duration 20 min, on a Waters 2795 instrument fitted with a 2996 DAD detector) and manual collection of the corresponding peak.

MS: [M+H]⁺=450.2.

### EXAMPLE 36

### 1-(2,3-Dihydrobenzo[1,4]dioxin-5-yl)-4-[3-(5-methylisoxazol-3-yl)-3-phenylpropyl]piperazine

### 4,4-Dimethoxy-2-phenylbutyraldehyde (Compound 36a)

This compound was prepared as described in WO 03/106443 A1.

### 7,7-Dimethoxy-5-phenylhept-2-yn-4-ol (Compound 36b)

Into a solution of 1.5 g of Compound 36a in 40 mL of anhydrous THF under a N₂ stream was dropped at r.t. a 0.5 M solution of 1-propynylmagnesium bromide in THF (18.6 mL). The reaction mixture was stirred at r.t. for 3 h. Afterwards, it was quenched with an aq. saturated solution of NH₄Cl and extracted with EtOAc (3×25 mL). The combined organic extracts were washed with water (2×25 mL), dried (Na₂SO₄) and evaporated to dryness in vacuo, affording 1.78 g (99.4%) of the title product that was used without further purification in the next step.

¹H-NMR (CDCl₃, δ): 1.83, 1.85 (2s, 3H), 1.93 (d, 1H), 1.95-2.12 (m, 1H), 2.20-2.42 (m, 1H), 2.98-3.10 (m, 1H), 3.28, 3.30, 3.31 (3s, 6H), 4.20-4.28 (m, 1H), 4.42-4.60 (m, 1H), 7.25-7.40 (m, 5H).

MS: [M+H]⁺=249.3.

### 7,7-Dimethoxy-5-phenylhept-2-yn-4-one (Compound 36c)

To a solution of 1.06 mL of oxalyl chloride in 40 mL of anhydrous CH₂Cl₂, cooled at −50° C., was added dropwise 1.72 mL of DMSO (at such rate to maintain the same temperature). The mixture was stirred for 15 minutes; afterwards, was added a solution of 2.0 g of Compound 36b in 25 mL of anhydrous CH₂Cl₂ and after 10 minutes, triethylamine (6.76 mL). The reaction was brought to r.t. by spontaneous heating. After overnight resting, it was quenched with water, the layers were separated and the aqueous layer was extracted with 2×15 mL of CH₂Cl₂. The combined organic extracts were washed with water (2×30 mL), dried (Na₂SO₄) and evaporated to dryness. The crude product was purified by flash chromatography (petroleum ether/EtOAc=8:2) affording the title compound (88.4%) as a colourless oil.

¹H-NMR (CDCl₃, δ): 1.96 (s, 3H), 1.98-2.08 (m, 1H), 2.48-2.58 (m, 1H), 3.29, 3.32 (2s, 6H), 3.90 (t, 1H), 4.25 (t, 1H), 7.22-7.43 (m, 5H).

MS: [M+H]⁺=247.3.

### 4-Oxo-3-phenylhept-5-ynal (Compound 36d)

The title compound was synthesised following the procedure reported for Compound 27b, but using as starting material Compound 36c instead of the Compound 27a. Yield: 97.2%.

MS: [M+H]⁺=201.2.

### 7-[4-(2,3-Dihydrobenzo[1,4]dioxin-5-yl)piperazin-1-yl]-5-phenylhept-2-yn-4-one (Compound 36e)

The title product was obtained by the same procedure described for the Compound of Example 12, but using the Compound 36d instead of acetone and 1-(2,3-dihydrobenzo[1,4]dioxin-5-yl)-piperazine instead of 5a. The crude product was purified by flash chromatography (petroleum ether-EtOAc=4:6) affording the title compound (39.6%) as a glassy oil.

¹H-NMR (CDCl₃, δ): 1.85-2.05 (m, 1H), 2.05 (s, 3H), 2.30-2.40 (t, 2H), 2.40-2.52 (m, 1H), 2.53-2.75 (m, 4H), 3.00-3.20 (m, 4H), 3.86 (t, 1H), 4.25-4.40 (m, 4H), 6.55 (dd, 1H), 6.61 (dd, 1H), 6.80 (t, 1H), 7.20-7.50 (m, 5H).

MS: [M+H]⁺=405.5.

### 1-(2,3-Dihydrobenzo[1,4]dioxin-5-yl)-4-[3-(5-methylisoxazol-3-yl)-3-phenylpropyl]piperazine

The title compound was prepared using the method described for the Compound of Example 1 but using as starting material Compound 36e instead of Compound 1a. After the initial 3 h at reflux, TLC analysis showed that the starting material 36e was totally consumed. Usual work-up and purification first by flash chromatography (CH₂Cl₂-MeOH=95:5) and then by preparative LC-MS chromatography [Xterra MS-C18 (19×100 mm, 5 μm) column; eluent mixture: buffer aq. NH₄HCO₃ 20 mM at pH 8/(CH₃CN:MeOH 1:1)=60:40, flow rate 20 ml/min, on a ZQ MS platform instrument fitted with a 2996 DAD detector] afforded 9.0% of the title product and 6.4% of the by-product 1-(2,3-dihydrobenzo[1,4]dioxin-5-yl)-4-[3-(3-methylisoxazol-5-yl)-3-phenylpropyl]piperazine.

¹H-NMR (CDCl₃, δ): 2.23-2.30 (m, 1H), 2.35 (s, 3H), 2.38-2.48 (m, 3H), 2.50-2.75 m, 4H), 3.00-3.20 (m, 4H), 4.18 (t, 1H), 4.25-4.45 (m, 4H), 5.78 (s, 1H), 6.55 (dd, 1H), 6.65 (dd, 1H), 6.79 (t, 1H), 7.23-7.40 (m, 5H).

MS: [M+H]⁺=420.5.

### EXAMPLE 37

### N-[1-Cyclohexyl-4-[4-(2,3-dihydrobenzo[1,4]dioxin-5-yl)piperazin-1-yl]-2-(2-fluorophenyl)butyl]formamide

### 1-Cyclohexyl-4-[4-(2,3-dihydrobenzo[1,4]dioxin-5-yl)piperazin-1-yl]-2-(2-fluorophenyl)butan-1-one (Compound 37a)

The title compound was synthesised following the procedure reported for the Compound of Example 12, but using 1-(2,3-dihydrobenzo[1,4]dioxin-5-yl)piperazine instead of 5a and 1-cyclohexyl-4,4-diethoxy-2-(2-fluorophenyl)butan-1-one (described as Compound 1c in US20040072839) instead of acetone and avoiding the use acetic acid. After the usual work-up procedure, the crude product was purified by flash chromatography (petroleum ether-EtOAc-1.6 N methanolic NH₃=85:15:2) affording the title compound as a colourless oil (81%).

¹H-NMR (CDCl₃, δ): 1.08-1.42 (m, 5H), 1.48-1.75 (m, 5H), 1.75-1.95 (m, 3H), 2.35-2.45 (m, 2H), 2.50-2.80 (m, 4H), 3.00-3.30 (m, 4H), 4.20-4.45 (m, 5H), 6.55 (dd, 1H), 6.61 (dd, 1H), 6.78 (t, 1H), 7.00-7.15 (m, 2H), 7.15-7.30 (m, 2H).

MS: [M+H]⁺=467.12

### N-[1-Cyclohexyl-4-[4-(2,3-dihydrobenzo[1,4]dioxin-5-yl)piperazin-1-yl]-2-(2-fluorophenyl)butyl]formamide

The title compound was synthesised following the procedure reported for the Compound of Example 6, but using an open vessel microwave oven (CEM Discover) and compound 37a instead of Compound 1a. To the reaction mixture was added a saturated solution of K₂CO₃; then it was extracted with CH₂Cl₂ (3×15 mL). The combined extracts were dried on Na₂SO₄, evaporated to dryness in vacuo to afford a crude, which was purified by column flash chromatography (petroleum ether-Et₂O-1.6 N methanolic NH₃ 6:4:0.4) to yield the pure title compound (50.1%), containing a 75:25 mixture of RS,SR and RS, RS diastereomers

¹H-NMR (DMSO-d₆, δ) 0.80-1.30 (m, 6H), 1.35-1.50 (m, 1H), 1.50-1.80 (m, 6H), 1.95-2.20 (m, 2H), 2.20-2.50 (m, 4H), 2.82-3.00 (m, 4H), 3.20-3.40 (m, 1H), 4.05-4.30 (m, 5H), 6.35, 6.50 (2dd, 2H), 6.70 (t, 1H), 7.05-7.35 (m, 4H), 7.50 (d, 1H), 7.85 (s, 1H).

MS: [M+H]⁺=496.14

### EXAMPLE 38

### (RS,SR)-1-Cyclohexyl-4-[4-(2,3-dihydrobenzo[1,4]dioxin-5-yl)-piperazin-1-yl]-2-(2-fluorophenyl)butylamine

To a solution of the compound of Example 37 (0.085 g) in EtOH (2 mL) and H₂O (0.5 mL) was added 2M HCl (0.51 mL) and the resulting mixture was heated in a microwave oven (3 cycles of 20 min. at 120° C.). Afterwards, the mixture was diluted with H₂O and extracted with CH₂Cl₂ (3×15 ml). The combined organic layers were dried on Na₂SO₄ and evaporated to dryness in vacuo to afford the title product as a 75:25 diastereomeric (RS,SR)— (RS,RS) mixture as revealed by LC-MS. Purification by flash chromatography (petroleum ether-Et₂O-1.6 N methanolic NH₃ 6:4:0.34) yielded the pure (RS,SR) isomer (30%).

1H-NMR (DMSO-d₆, δ) 0.80-1.35 (m, 6H), 1.50-1.82 (m, 6H), 1.80-1.95 (m, 1H), 1.95-2.05 (m, 1H), 2.05-2.20 (m, 1H), 2.25-2.45 (m, 4H), 2.65 (t, 1H), 2.80-3.00 (m, 4H), 3.05-3.15 (m, 1H), 3.20-3.40 (br, 2H), 4.15-4.25 (m, 4H), 6.42 (dd, 1H), 6.49 (dd, 1H), 6.70 (dd, 1H), 7.05-7.30 (m, 3H), 7.45 (t, 1H).

MS: [M+H]+=468.25.

### EXAMPLE 39

### 1-Cyclohexyl-4-[4-(2,3-dihydrobenzo[1,4]dioxin-5-yl)piperazin-1-yl]-2-(2-fluorophenyl)butylmethylamine

The title compound was obtained as described for the Compound of Example 10—method B but using as a starting material the compound of Example 37 instead of the compound of Example 6. After the usual work-up procedure, the title product was obtained as a colourless oil (86.3%).

1H-NMR (DMSO-d₆, δ): 1.00-1.20 (m, 6H), 1.30-1.70 (m, 6H), 1.75-1.85 (m, 1H), 1.86-1.95 (m, 1H), 1.96-2.05 (m, 1H), 2.06-2.20 (m, 4H), 2.25-2.50 (m, 5H), 2.80-3.00 (m, 4H), 3.10-3.20 (m, 1H), 4.15-4.25 (m, 4H), 6.45 (dd, 1H), 6.48 (dd, 1H), 6.70 (dd, 1H), 7.05-7.30 (m, 3H), 7.50-7.60 (t, 1H)

MS: [M+H]+=482.24

### EXAMPLE 40

### N-[1-Cyclohexyl-4-[4-(2,3-dihydrobenzo[1,4]dioxin-5-yl)piperazin-1-yl]-2-(2-fluorophenyl)butyl]acetamide

The title product was synthesised following the procedure described for the Compound of Example 3, but using as a starting material the Compound 38 instead of the Compound of Example 1. Purification by preparative HPLC afforded 61% of the title product.

1H-NMR (DMSO-d₆, δ): 0.80-1.50 (m, 6H), 1.50-1.80 (m, 9H), 1.80-1.90 (m, 1H), 2.00-2.20 (m, 2H), 2.25-2.50 (m, 4H), 2.80-3.00 (m, 4H), 3.20-3.40 (m, 1H), 4.00-4.10 (m, 1H), 4.15-4.30 (m, 4H), 6.45 (dd, 1H), 6.49 (dd, 1H), 6.70 (dd, 1H), 7.00-7.35 (m, 5H).

MS: [M+H]+=510.22

### EXAMPLE 41

### N-[1-Cyclohexyl-4-[4-(2,3-dihydrobenzo[1,4]dioxin-5-yl)piperazin-1-yl]-2-(2-fluoro-phenyl)butyl]-N-methylacetamide

The title product was synthesised following the procedure described for the Compound of Example 3, but using as a starting material Compound 39 instead of the Compound of Example 1. Purification by preparative HPLC afforded 56% of the title compound.

1H-NMR (DMSO-d₆, δ): 0.90-1.40 (m, 6H), 1.50-2.20 (m, 12H), 2.05-2.20 (m, 1H), 2.30-2.55 (m, 6H), 2.80-3.00 (m, 4H), 3.40-3.60 (m, 1H), 3.90-4.00 (m, 0.4H), 4.10-4.30 (m, 4H), 4.70-4.90 (m, 0.6H), 6.42 (dd, 1H), 6.49 (dd, 1H), 6.70 (dd, 1H), 7.05-7.50 (m, 4H).

MS: [M+H]+=524.38.

### EXAMPLE 42

### N-[4-[4-(2-Bromo-5-methoxybenzyl)piperazin-1-yl]-1-cyclohexyl-2-(2-fluorophenyl)butyl]formamide

### 4-[4-(2-Bromo-5-methoxybenzyl)piperazin-1-yl]-1-cyclohexyl-2-(2-fluorophenyl)butan-1-one (Compound 42a)

The title compound was synthesised following the procedure reported for the Compound of Example 12, but using 1-(2-bromo-5-methoxybenzyl)piperazine instead of 5a and 1-cyclohexyl-4,4-diethoxy-2-(2-fluorophenyl)butan-1-one (described as Compound 1c in patent US20040072839) instead of acetone. After the usual work-up procedure, the crude product was purified by flash chromatography (petroleum ether-EtOAc=1:1) affording the title compound as a colourless oil (0.21 g; 78.7%).

1H-NMR (CDCl₃, δ): 0.90-1.40 (m, 5H), 1.50-2.00 (m, 7H), 2.20-2.70 (m, 11H), 3.60 (s, 2H), 3.81 (s, 3H), 4.35 (t, 1H), 6.70 (dd, 1H), 7.05-7.30 (m, 5H), 7.43 (d, 1H).

MS: [M+H]+=532.09

### N-[4-[4-(2-Bromo-5-methoxybenzyl)piperazin-1-yl]-1-cyclohexyl-2-(2-fluorophenyl)butyl]formamide

The title compound was synthesised following the procedure reported for the Compound of Example 6, but using Compound 42a instead of Compound 1a. After the work-up procedure, the crude was purified by flash chromatography (petroleum ether-Et₂O-1.6 N methanolic NH₃ 6:4:0.4) giving the pure title compound (57.7%), used as an intermediate without further purification. A small amount was purified by preparative LC affording the pure title compound.

1H-NMR (DMSO-d₆, δ): 0.90-1.40 (m, 5H), 1.40-1.55 (m, 1H), 1.60-1.80 (m, 6H), 1.81-1.92 (m, 1H), 1.95-2.45 (m, 7H), 3.15-3.35 (m, 4H), 3.45 (s, 2H), 3.73 (s, 3H), 4.05-4.15 (m, 1H), 6.78 (dd, 1H), 7.00-7.60 (m, 7H), 7.85 (s, 1H).

MS: [M+H]+=561.14

### EXAMPLE 43

### 4-[4-(2-Bromo-5-methoxybenzyl)piperazin-1-yl]-1-cyclohexyl-2-(2-fluorophenyl)butylamine

The title product was synthesised following the procedure described for the Compound of Example 38, but using as a starting material the Compound 42 instead of the Compound of Example 37. The usual work-up afforded the title compound (85%), which was used for further transformation without additional purification. A sample was purified by preparative HPLC for analytical purposes.

1H-NMR (DMSO-d₆, δ): 0.90-1.35 (m, 6H), 1.50-1.80 (m, 6H), 1.81-2.15 (m, 3H), 2.16-2.45 (m, 8H), 2.67 (t, 1H), 3.00-3.10 (m, 1H), 3.20-3.40 (br, 2H), 3.45 (s, 2H), 3.73 (s, 3H), 6.80 (dd, 1H), 7.00-7.25 (m, 4H), 7.38-7.50 (m, 2H)

MS: [M+H]+=533.16

### EXAMPLE 44

### 4-[4-(2-Bromo-5-methoxybenzyl)piperazin-1-yl]-1-cyclohexyl-2-(2-fluorophenyl)butylmethylamine

The title compound was obtained as described for the Compound of Example 10—method B but using as a starting material the compound of Example 42, instead of the compound of Example 6. After the usual work-up procedure, the title product was purified by flash chromatography (petroleum ether-Et₂O-1.6 N methanolic NH₃ 7:3:0.2) affording the title compound (62.5%)

1H-NMR (DMSO-d₆, δ): 0.80-1.90 (m, 13H), 1.90-2.00 (m, 1H), 2.00-2.12 (m, 1H), 2.14 (s, 3H), 2.20-2.45 (m, 9H), 3.05-3.15 (m, 1H), 3.20-3.40 (br, 1H), 3.45 (s, 2H), 3.73 (s, 3H), 6.80 (dd, 1H), 7.00-7.25 (m, 4H), 7.40-7.55 (m, 2H)

MS: [M+H]+=547.26

### EXAMPLE 45

### N-[4-[4-(2-Bromo-5-methoxybenzyl)piperazin-1-yl]-1-cyclohexyl-2-(2-fluoro-phenyl)butyl]acetamide

The title product was synthesised following the procedure described for the Compound of Example 3, but using as a starting material Compound 43 instead of the Compound of Example 1. Purification by preparative HPLC afforded 63% of the title product.

1H-NMR (DMSO-d₆, δ): 0.90-1.30 (m, 5H), 1.32-1.45 (m, 1H), 1.52-1.75 (m, 9H), 1.75-1.90 (m, 1H), 1.95-2.15 (m, 2H), 2.16-2.48 (m, 8H), 3.20-3.35 (m, 1H), 3.46 (s, 2H), 3.73 (s, 3H), 3.95-4.10 (m, 1H), 6.80 (dd, 1H), 7.00-7.30 (m, 6H), 7.46 (d, 1H)

MS: [M+H]+=575.14.

### EXAMPLE 46

### N-[4-[4-(2-Bromo-5-methoxybenzyl)piperazin-1-yl]-1-cyclohexyl-2-(2-fluorophenyl)butyl]-N-methylacetamide

The title product was synthesised following the procedure described for the Compound of Example 3, but using as a starting material Compound 44 instead of the Compound of Example 1. After the usual work-up procedure, the title product was purified by flash chromatography (petroleum ether-Et₂O-1.6 N methanolic NH₃ 8:2:0.2) affording the title compound (90.8%).

1H-NMR (DMSO-d₆, δ): 0.80-1.50 (m, 6H), 1.52-2.00 (m, 12H), 2.02-2.15 (m, 1H), 2.16-2.48 (m, 11H), 3.40-3.50 (m, 2H), 3.73 (s, 3H), 3.90-4.00 (m, 0.36H), 4.75-4.85 (m, 0.64H), 6.80 (dd, 1H), 7.00-7.42 (m, 5H), 7.46 (d, 1H)

MS: [M+H]+=589.24

### EXAMPLE 47

### N-[4-[4-(1H-Benzimidazol-4-yl)piperazin-1-yl]-1-cyclohexyl-2-(2-fluoro-phenyl)butyl]formamide

### 4-[4-(1H-Benzimidazol-4-yl)piperazin-1-yl]-1-cyclohexyl-2-(2-fluorophenyl)butan-1-one (Compound 47a)

The title compound was synthesised following the procedure reported for the Compound of Example 12, but using 1-(1H-benzimidazol-4-yl)piperazine instead of 5a and 1-cyclohexyl-4,4-diethoxy-2-(2-fluorophenyl)butan-1-one (described as Compound 1c in patent US20040072839) instead of acetone. After the usual work-up procedure, the crude product was purified by flash chromatography (petroleum ether-EtOAc=1:1) affording the title compound as a colourless oil (0.21 g; 78.7%).

1H-NMR (CDCl₃, δ): 1.10-1.90 (m, 10H), 1.92-2.00 (m, 1H), 2.30-2.45 (m, 4H), 2.60-2.75 (m, 4H), 3.30-3.80 (br, 4H), 4.35-4.45 (m, 1H), 6.60. 6.85 (m, 1H), 7.00-7.35 (m, 6H), 7.98 (s, 1H), 9.30-9.60 (br, 1H).

MS: [M+H]+=449.29

### N-[4-[4-(1H-Benzimidazol-4-yl)piperazin-1-yl]-1-cyclohexyl-2-(2-fluorophenyl)butyl]formamide

The title compound was synthesised following the procedure reported for the Compound of Example 6, but using Compound 47a instead of Compound 1a. Purification of the crude after the usual work-up procedure (petroleum ether-Et₂O-1.6 N methanolic NH₃ gradient from 3-7-0.7 to 1-9-0.9) afforded the title compound (47%)

1H-NMR (DMSO-d₆, δ): 0.90-1.40 (m, 5H), 1.40-1.80 (m, 6H), 1.85-1.98 (m, 1H), 2.00-2.30 (m, 2H), 2.30-2.60 (m, 4H), 2.95-3.10 (m, 1H), 3.20-3.35 (m, 1H), 3.36-3.60 (m, 4H), 4.05-4.20 (m, 1H), 6.45 (dd, 1H), 6.95-7.60 (m, 7H), 7.89 (s, 1H) 8.02 (s, 1H), 12-30 (s, 1H).

MS: [M+H]+=478.18

### EXAMPLE 48

### 4-[4-(1H-Benzimidazol-4-yl)piperazin-1-yl]-1-cyclohexyl-2-(2-fluorophenyl)butylamine

The title product was synthesised following the procedure described for the Compound of Example 38, but using as a starting material Compound 47 instead of the Compound of Example 37. The usual work-up afforded the title compound (99%).

1H-NMR (DMSO-d₆, δ): 0.90-1.40 (m, 5H), 1.50-1.85 (m, 7H), 1.85-2.00 (m, 1H), 2.00-2.10 (m, 1H), 2.12-2.35 (m, 1H), 2.36-2.55 (m, 4H), 2.60-2.75 (m, 1H), 3.05-3.50 (m, 7H), 6.50 (dd, 1H), 7.00-7.30 (m, 5H), 7.48 (t, 1H), 8.02 (s, 1H), 12.30 (s, 1H).

MS: [M+H]+=450.21

### Example 48a

### (RS,SR)-4-[4-(1H-Benzimidazol-4-yl)piperazin-1-yl]-1-cyclohexyl-2-(2-fluorophenyl)butylamine

### Example 48b

### (RS,RS)-4-[4-(1H-Benzimidazol-4-yl)piperazin-1-yl]-1-cyclohexyl-2-(2-fluorophenyl)butylamine

For a larger scale preparation of the Compound of Example 48, the crude product was purified by flash chromatography eluting first by petroleum ether-Et₂O-1.6 N methanolic ammonia 45:5:6.5, followed by petroleum ether-Et₂O-1.6 N methanolic ammonia 15:85:0.85, affording Compound 48a (35%) as the firstly eluted product followed by Compound 48b (15%).

### EXAMPLE 49

### 4-[4-(1H-Benzimidazol-4-yl)piperazin-1-yl]-1-cyclohexyl-2-(2-fluorophenyl)butylmethylamine

The title compound was obtained as described for the Compound of Example 10—method B but using as a starting material the Compound of Example 47, instead of the compound of Example 6. After the usual work-up procedure, the title product was purified by flash chromatography (petroleum ether-AcOEt-1.6 N methanolic NH₃ 5:5:0.5) affording the title compound (43.5%), which was used for further transformation without additional purification. A sample was purified by preparative HPLC for analytical purposes.

MS: [M+H]+=464.32

### EXAMPLE 50

### N-[4-[4-(1H-Benzimidazol-4-yl)piperazin-1-yl]-1-cyclohexyl-2-(2-fluorophenyl)butyl]acetamide

The title product was synthesised following the procedure described for the Compound of Example 3, but using as a starting material the Compound 48 instead of the Compound of Example 1. Purification by preparative HPLC afforded 18.9% of the title product.

1H-NMR (DMSO-d₆, δ): 0.85-1.35 (m, 5H), 1.35-1.50 (m, 1H), 1.53-1.80 (m, 9H), 1.85-1.98 (m, 1H), 2.00-2.30 (m, 2H), 2.35-2.55 (m, 4H), 3.20-3.60 (m, 5H), 4.00-4.15 (m, 1H), 6.45-6.60 (br, 1H), 6.85-7.40 (m, 7H), 8.02 (s, 1H), 12.30 (s, 1H).

MS: [M+H]+=492.24

### EXAMPLE 51

### N-[4-[4-(1H-Benzimidazol-4-yl)piperazin-1-yl]-1-cyclohexyl-2-(2-fluorophenyl)butyl]-N-methylacetamide

The title product was synthesised following the procedure described for the Compound of Example 3, but using as a starting material Compound 49 instead of the Compound of Example 1. Purification by preparative HPLC afforded 54% of the title product.

1H-NMR (DMSO-d₆, δ): 0.90-1.40 (m, 6H), 1.60-2.12 (m, 11H), 2.10-2.30 (m, 1H), 2.30-2.50 (m, 7H), 3.00 (bs, 0.75H), 3.35-3.60 (m, 4.25H), 3.95-4.05 (m, 0.36H), 3.75-3.85 (m, 0.65H), 6.45 and 6.70 (2dd, 1H), 6.95-7.50 (m, 6H), 8.02 and 8.10 (2s, 1H), 12.30 (s, 1H).

MS: [M+H]+=506.25

### EXAMPLE 52

### N-{1-Cyclohexyl-2-(2-fluorophenyl)-4-[4-(1H-indol-4-yl)piperazin-1-yl]butyl}formamide

### 1-Cyclohexyl-2-(2-fluorophenyl)-4-[4-(1H-indol-4-yl)piperazin-1-yl]butan-1-one (Compound 52a)

The title compound was synthesised following the procedure reported for the Compound of Example 12, but using 1-(1H-indol-4-yl)piperazine instead of 5a and 1-cyclohexyl-4,4-diethoxy-2-(2-fluorophenyl)butan-1-one (described as Compound 1c in patent US20040072839) instead of acetone. After the usual work-up procedure, the crude product was purified by flash chromatography (petroleum ether-AcOEt-1.6 N methanolic NH₃ 8:2:0.2) affording the title compound (64.6%).

¹H-NMR (CDCl₃, δ): 1.08-1.45 (m, 5H), 1.48-2.00 (m, 8H), 2.30-2.50 (m, 2H), 2.60-2.80 (m, 4H), 3.20-3.40 (m, 4H), 4.35-4.50 (m, 1H), 6.56 (t, 1H), 6.62 (d, 1H), 7.05-7.30 (m, 7H), 8.20 (s, 1H).

MS: [M+H]+=448.11

### N-{1-Cyclohexyl-2-(2-fluorophenyl)-4-[4-(1H-indol-4-yl)piperazin-1-yl]butyl}formamide

The title compound was synthesised following the procedure reported for the Compound of Example 6, but using Compound 52a instead of Compound 1a. Purification by preparative LC of the crude after the usual work-up procedure afforded the title compound (8.9%)

1H-NMR (DMSO-d₆, δ): 0.80-1.45 (m, 5H), 1.36-2.60 (m, 14H), 2.90-3.20 (m, 4H), 3.20-3.40 (m, 1H), 4.05-4.20 (m, 1H), 6.30-6.40 (m, 1H), 6.52 (d, 1H), 6.70 (d, 1H), 6.90-7.50 (m, 7H), 7.85 (s, 1H), 11.00 (s, 1H)

MS: [M+H]+=399.5

### EXAMPLE 53

### 1-{(RS,SR)-1-Cyclohexyl-2-(2-fluorophenyl)-4-[4-(2-methoxyphenyl)piperazin-1-yl]butyl}-1,3,3-trimethylthiourea

Into a solution of the Compound of Example 10a (0.09 g), DIPEA (78 μL) in 3 mL of CH₂Cl₂ was dropped over 1 h at r.t. a solution of 97.3 μL of thiofosgene in 2 mL of CH₂Cl₂. After 2 h under stirring, 0.655 μL of a 0.56 M solution of dimethylamine in toluene was added followed by DIPEA (78 μL). After overnight stirring, the reaction mixture was quenched with water, basified by aq. NaOH and extracted with dichloromethane. The crude was purified by flash chromatography (CHCl₃) followed by a further flash column eluted with petroleum ether EtOAc 7:3 giving 0.045 mg of the title product (42%).

¹H-NMR (CDCl₃, δ): 1.10-2.40 (m, 15H), 2.41-2.70 (m, 10H), 2.73 (s, 3H), 2.90-3.20 (m, 4H), 3.50-3.65 (m, 1H), 3.86 (s, 3H), 5.35-5.55 (m, 1H), 6.80-7.20 (m, 7H), 7.40-7.50 m, 1H).

MS: [M+H]+=541.87

### EXAMPLE 54

### Radioligand and binding to recombinant 5-HT_{1A} receptor

A. Affinity

Genomic clone G-21 encoding the human 5HT_{1A}-serotonergic receptor was stably transfected in a human cell line (HeLa). HeLa cells were grown as monolayers in Dulbecco's modified Eagle medium (DMEM), containing 10% fetal bovine serum and gentamycin (100 μg/ml) at 37° C. in a humidified atmosphere of 5% CO₂ and air. The cells were detached from the growth flask at 95% confluence by a cell scraper and were lysed in cold 5 mM Tris and 5 mM EDTA buffer (pH 7.4). The homogenates were centrifuged at 40000×g×20 minutes and the pellets were resuspended in a small volume of cold 5 mM Tris and 5 mM EDTA buffer (pH 7.4) and immediately frozen and stored at −70° C. until use. On the day of experiment, the cell membranes were resuspended in incubation buffer: 50 mM Tris (pH 7.4), 2.5 mM MgCl₂, 10 μM pargyline (Fargin et al., Nature 335, 358-360, 1988). The membranes were incubated in a final volume of 0.32 ml for 30 minutes at 30° C. with 1 nM [³H]8-OH-DPAT, in the absence or presence of the test compounds. Non-specific binding was determined in the presence of 10 μM 5-HT. Incubation was stopped by addition of cold Tris buffer and rapid filtration through UniFilter GF/C plate.

B. Functional Activity

[³⁵S]GTPγS Binding.

On the date of the experiment, cell membranes from HeLa cells transfected with human cloned 5-HT_{1A} receptors were resuspended in buffer pH 7.4 containing 20 mM HEPES, 3 mM MgCl₂ and 120 mM NaCl (Stanton, J. A.; Beer, M. S. Eur. J. Pharmacol. 320, 267-275, 1997). The membranes were incubated with 10 μM GDP and decreasing concentrations of test drugs (from 100 μM to 0.1 nM) or decreasing concentrations of 5-HT (from 100 μM to 0.1 nM, reference curve) for 20 min at 30° C. in a final volume of about 0.25 ml. [³⁵S]GTPγS (200-250 pM in 10 μl) was added to samples and incubated for a further 30 min at 30° C. Non-specific binding was determined in the presence of 10 μM GTPγS. The incubation was stopped by addition of ice-cold HEPES buffer and rapid filtration on Unifilter GF/C filters, using a Filtermate cell harvester (Packard). The filters were washed four times with total 1.2 ml of same buffer. Radioactivity was counted by liquid scintillation spectrometry with efficiency >90% (TopCount Packard). Stimulation of [³⁵S]GTPγS binding induced by the compounds tested was expressed as % increase in binding above basal value, being the maximal stimulation observed with 5-HT taken as 100%. The concentration-response curve of the agonistic activity was analyzed by non linear fitting program (De Lean et al., Am. J. Physiol. 235, E97-E102, 1978).

A stimulation of [³⁵S]GTPγS binding represented the functional correlate of the binding of an agonist compound at the 5-HT_{1A} receptors. Stimulation induced by the endogenous ligand 5-HT was considered the maximal stimulation attainable. Compounds stimulating [³⁵S]GTPγS binding at a lower level were considered as partial agonists. Compounds that did not stimulate [³⁵S]GTPγS binding were considered as neutral antagonists. Compounds that reduced [³⁵S]GTPγS binding were considered as inverse agonists.

C. Results

The affinity of the tested compounds was evaluated as inhibition of specific binding of the radioligand to 5-HT_{1A} receptor (IC₅₀) by using the non-linear curve-fitting program Allfit (De Lean et al., Am. J. Physiol. 235, E97-E102 (1978)). The IC₅₀ value was converted to an affinity constant (Ki) by the equation of Cheng et al., Biochem. Pharmacol. 22, 3099-3108 (1973).

The results reported in Table 1 show that the compounds of the invention have a very high affinity for the 5-HT_{1A} receptor. In addition, all tested compounds behaved as neutral antagonists (NA) or inverse agonists (IA) at the 5-HT1A receptor.

TABLE 1
Binding affinity for 5HT_{1A} receptor

| | | Functional |
|---|---|---|
| Example | Ki (nM) | Activity |
| 1 | 0.16 | NA |
| 2 | 1.9 | NA |
| 3 | 0.87 | NA |
| 4 | 0.88 | NA |
| 4a | 0.68 | IA |
| 5a | 0.48 | NA |
| 5b | 0.77 | NA |
| 5aA | 0.6 | NA |
| 5aB | 1.20 | NA |
| 6 | 1.2 | NA |
| 7 | 1.69 | IA |
| 8 | 0.68 | IA |
| 9 | 1.4 | NA |
| 10a | 0.49 | NA |
| 10b | 1.41 | NA |
| 11 | 1.0 | NA |
| 12 | 0.99 | NA |
| 13 | 0.3 | IA |
| 14 | 0.42 | NA |
| 15 | 7.28 | NA |
| 16 | 0.86 | NA |
| 17 | 0.68 | IA |
| 18 | 1.84 | NA |
| 19 | 2.09 | NA |
| 20 | 2.9 | NA |
| 21 | 2.1 | NA |
| 22 | 1.24 | NA |
| 23 | 0.58 | NA |
| 24 | 1.23 | NA |
| 24A | 0.7 | IA |
| 24B | 4.2 | NA |
| 26 | 0.38 | NA |
| 27 | 1.77 | |
| 28 | 1.33 | NA |
| 31 | 6.03 | NA |
| 32 | 2.24 | NA |
| 37 | 0.8 | NA |
| 38 | 0.8 | NA |
| 39 | 0.3 | NA |
| 41 | 0.9 | IA |
| 42 | 0.5 | NA |
| 43 | 0.3 | NA |
| 44 | 0.4 | NA |
| 45 | 1.1 | IA |
| 46 | 0.8 | NA |
| 47 | 0.8 | NA |
| 48 | 0.3 | NA |
| 52 | 2.3 | NA |
| 53 | 1.7 | IA |

### EXAMPLE 55

### Inhibition of Serotonergic Syndrome (Forepaw Treading) Induced by 8-OH-DPAT in Rats (Post-Synaptic Antagonism)

A. Method:

The inhibitory effect of 5-HT_{1A}-receptor antagonists on stereotyped forepaw treading induced in rats by subcutaneous injection of 8-OH-DPAT was evaluated by the method of Tricklebank (Tricklebank et al., Eur. J. Pharmacol., 117: 15, 1985) with minor modifications as described below.

Male Sprague-Dawley rats [Crl: CD® (SD) IGS BR] weighing 150-175 g from Charles River Italia were used. The animals were housed with free access to food and water and maintained on a forced 12-hour-light/12-hour-dark cycle at 22-24° C. of temperature. On the day of the experiment, the rats were placed singly in clear plastic containers, 10-15 minutes before oral administration of the vehicle or compounds to be tested. For evaluation of antagonistic activity, the compounds were administered 1 and 4 hours before induction of stereotypy by 8-OH-DPAT (1 mg/kg subcutaneously). Observation sessions lasted 30 seconds and began 3 minutes after 8-OH-DPAT treatment and were repeated every 3 minutes over a period of 15 minutes.

The appearance of the symptoms induced by postsynaptic stimulation of 5-HT_{1A} receptors was noted, and the intensity was scored using an intensity scale in which: 0=absent, 1=equivocal, 2=present and 3=intense. Behavioural scores for treated rats were accumulated throughout the observation time (5 observation periods) and expressed as mean values of 4 rats/dose. Change in mean values of treated animals in comparison with control (vehicle) group, expressed as per-cent inhibition, was used to quantify the antagonistic activity.

B. Results:

The results are shown in Table 2. These results demonstrate that administration of the compounds of the invention (e.g., the compound of Example 24) lead to significant and long-lasting post-synaptic 5-HT_{1A}-receptor antagonist activity. In order to quantitate the potency of the compound of Example 24 in inhibiting the stereotypy (forepaw treading) induced by 8-OH-DPAT, an ED₅₀ value (dose inhibiting by 50% the forepaw treading) was evaluated by means of linear regression using the least square method. Considering the results obtained 1 and 4 hours after the oral administration of different doses of the compound, the ED₅₀ values (95% CL) resulted 2.86 (2.12-3.8) and 3.67 (2.81-4.93) mg/kg p.o., respectively.

TABLE 2
Inhibition of forepaw treading induced by 8-OH-DPAT
in rats (post-synaptic antagonism)

| | |
|---|---|
| Dose | % Inhibition of forepaw treading |

| Compound | (mg/kg p.o.) | 1 h | 4 h |
|---|---|---|---|
| Ex. 1 | 10 | 71 | |
| Ex. 4 | 10 | 86 | 68 |
| Ex. 4b | 10 | 71 | 67 |
| Ex. 7 | 10 | 87 | 86 |
| Ex. 11 | 10 | 56 | 56 |
| Ex. 16 | 10 | 80 | 69 |
| Ex. 17 | 10 | 75 | 92 |
| Ex. 21 | 10 | 94 | 65 |
| Ex. 24 | 10 | 100 | 90 |

### EXAMPLE 56

### Effects on Rhythmic Bladder-Voiding Contractions Induced by Bladder Filling in Anaesthetised Rats

A. Method:

Female Sprague-Dawley rats weighing 225-275 g (Crl: CD® (SD) IGS BR, Charles River Italia) were used. The animals were housed with free access to food and water and maintained on a forced 12-hour alternating light-dark cycle at 22-24° C. for at least one week, except during the experiment. The activity on rhythmic bladder voiding contractions was evaluated according to the method of Dray (Dray J., Pharmacol. Methods, 13:157, 1985), with some modifications as in Guarneri (Guarneri, Pharmacol. Res. 27:173, 1993). Briefly, the rats were anaesthetised by subcutaneous injection of 1.25 g/kg (5 ml/kg) urethane, after which the urinary bladder was catheterised via the urethra using PE 50 polyethylene tubing filled with physiological saline. The catheter was tied in place with a ligature around the external urethral orifice and was connected to conventional pressure transducers (Statham P23 ID/P23 XL). The intravesical pressure was displayed continuously on a chart recorder (Battaglia Rangoni KV 135 with DCI/TI amplifier; Rectigraph-8K San-ei with BM 614/2 amplifier from Biomedica Mangoni). The bladder was then filled via the recording catheter by incremental volumes of warm (37° C.) saline until reflex bladder-voiding contractions occurred (usually 0.8-1.5 ml). For intravenous injection of bioactive compounds, PE 50 polyethylene tubing filled with physiological saline was inserted into the jugular vein.

From the cystometrogram, the number of contractions recorded 15 minutes before (basal values) and after treatment, as well as the mean amplitude of these contractions (mean height of the peaks in mmHg), was evaluated.

Since most compounds produced an effect that was relatively rapid in onset and led to a complete cessation of bladder contractions, bioactivity was conveniently estimated by measuring the duration of bladder quiescence (i.e., the time during which no contractions occurred). The number of tested animals showing a reduction in the number of contractions higher than 30% of that observed in the basal period was recorded too.

To compare the potency of the tested compounds in inhibiting the bladder voiding contractions, equieffective doses which resulted in the disappearance of contractions for a time of 10 minutes (ED₁₀ₘᵢₙ) were computed by means of linear regression using the least square method. The extrapolated doses which induced a reduction in the number of contractions greater than 30% in 50% of the treated rats (ED₅₀) were also evaluated by the method of Bliss (Bliss C. I., Quart J. Pharm. Pharmacol. 11, 192-216, 1938).

B. Results

The rapid distension of the urinary bladder in urethane-anaesthetised rats produced a series of rhythmic bladder-voiding contractions whose characteristics have been described (Maggi et al., Brain Res. 380:83, 1986; Maggi et al., J. Pharmacol. Exp. Ther., 230: 500, 1984). The frequency of these contractions is related to the sensory afferent arm of reflex micturition and to the integrity of the micturition centre, while their amplitude depends on the function of the reflex efferent arm. In this model system, compounds that act mainly on the central nervous system (such as morphine) cause a block in voiding contractions, whereas drugs that act at the level of the detrusor muscle, such as oxybutynin, lower the amplitude of the bladder contractions.

The results obtained after administration of prior-art compounds and a compound of the invention are shown in Table 3.

The compounds of the invention were superior to the reference standards in blocking the volume-induced rhythmic bladder contractions. Furthermore, the block of contractions by compounds of the invention was obtained without a decrease in their amplitude. Hence, compounds of the invention reduce urinary frequency without the undesirable side effect of retention of residual urine in the bladder after micturition, which can accompany a decrease in bladder contraction amplitude.

In comparison to compounds of the invention, oxybutynin had the undesirable effect of decreasing the amplitude of contractions in a dose-related manner, with an ED₅₀ value (the extrapolated dose inducing a 30% reduction of amplitude of the contractions in 50% of treated rats) of 240 μg/kg. At this dosage, oxybutynin did not lower the frequency of bladder contractions. The amplitude-reduction effect characteristic of oxybutynin, which can potentially cause lower bladder contractility and the undesirable retention of residual urine in the bladder after micturition, is not a characteristic of the compounds of the invention.

TABLE 3
Effects on rhythmic bladder-voiding
contractions after intravenous administration
Data represent the ED_{10 min} values (the extrapolated dose inducing
10 minutes of disappearance of the contractions), the ED₅₀
(frequency) values (the extrapolated doses inducing a reduction of
the number of contractions >30% in 50% of treated rats), and the
ED₅₀ (amplitude) values (the extrapolated doses inducing a 30%
reduction of amplitude of the contractions in 50% of treated rats).

| | ED_{10 min} | ED₅₀ (frequency) | ED₅₀ (amplitude) |
|---|---|---|---|
| Compound | μg/kg | μg/kg | μg/kg |
| Ex. 16 | 72 | 31 | n.a. |
| Flavoxate | >10000 | 2648 | n.a. |
| Oxybutynin | 7770 | >10000 | 240 |

n.a. = not active; no significant reduction of the height of the peaks

### EXAMPLE 57

### Effect on Cystometric Parameters in Conscious Rats After Oral Administration

A. Method:

Male Sprague-Dawley rats [Crl: CD® (SD) IGS BR] of 300-400 g body weight supplied by Charles River Italia were used. The animals were housed with free access to food and water and maintained on a forced 12-hour-light/12-hour-dark cycle at 22-24° C. of temperature, except during the experiment. To quantify urodynamic parameters in conscious rats, cystometrographic studies were performed according to the procedure previously reported (Guarneri et al., Pharmacol. Res. 24: 175, 1991).

Briefly, the rats were anaesthetised by intraperitoneal administration of 3 ml/kg of Equithensin solution (pentobarbital 30 mg/kg and chloral hydrate 125 mg/kg) and placed in a supine position. An approximately 10 mm-long midline incision was made in the shaved and cleaned abdominal wall. The urinary bladder was gently freed from adhering tissues, emptied and then cannulated via an incision in the bladder body, using a polyethylene cannula (0.58-mm internal diameter, 0.96-mm external diameter) which was permanently sutured with silk thread. The cannula was exteriorised through a subcutaneous tunnel in the retroscapular area, where it was connected to a plastic adapter in order to avoid the risk of removal by the animal. For drug testing, the rats were utilised one day after implantation.

On the day of the experiment, the rats were placed in modified Bollman cages, i.e., restraining cages that were large enough to permit the rats to adopt a normal crouched posture, but narrow enough to prevent turning around. After a stabilisation period of about 20 minutes, the free tip of the bladder cannula was connected through a T-shaped tube to a pressure transducer (Statham P23XL) and to a peristaltic pump (Gilson minipuls 2) for continues infusion of a warm (37° C.) saline solution into the urinary bladder, at a constant rate of 0.1 ml/minute. The intraluminal-pressure signal during infusion of saline into the bladder was continuously recorded on a polygraph (Rectigraph-8K San-ei with BM614/2 amplifier from Biomedica Mangoni). The cystometrogram was used to evaluate the urodynamic parameters of bladder volume capacity (BVC) and micturition pressure (MP). BVC (ml) is defined as the volume of saline infused into the bladder necessary to induce detrusor contraction followed by micturition. MP (mmHg) is defined as the maximal intravesical pressure caused by contraction during micturition. Basal BVC and MP values were evaluated as mean of the values observed in the cystometrograms recorded in an initial period of 30-60 minutes. Following determination of basal BVC and MP, the infusion was interrupted and the test compounds were administered orally by a stomach tube. Bladder infusion was resumed and changes in BVC and MP were evaluated from the mean values obtained in the cystometrograms observed during 1, 2, 3, 4 and 5 hours after treatment. Compounds were administered in a volume of 2 ml/kg and groups of control animals received the same amount of vehicle (0.5% methocel in water) orally.

Statistical Analysis

Data were expressed as mean ±standard error. The percent changes of BVC and MP versus the basal values, as well as Δ values (difference in ml or mmHg) of BVC and MP (BVC or MP at time “x” minus basal value), were also evaluated for each rat/time. In the figures, data were reported as % changes versus basal values.

Statistical analysis on BVC and MP values, as well as on Δ values, was performed by S.A.S./STAT software, version 6.12. The observed differences between vehicle (control) and test treatments were evaluated on Δ values of BVC and MP, whereas the differences between the values at different times versus basal values were analyzed on original BVC and MP data.

B. Results:

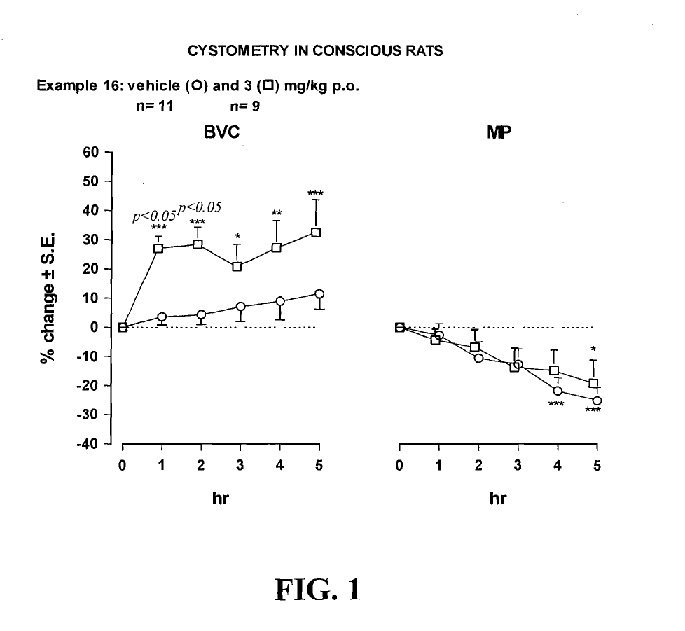

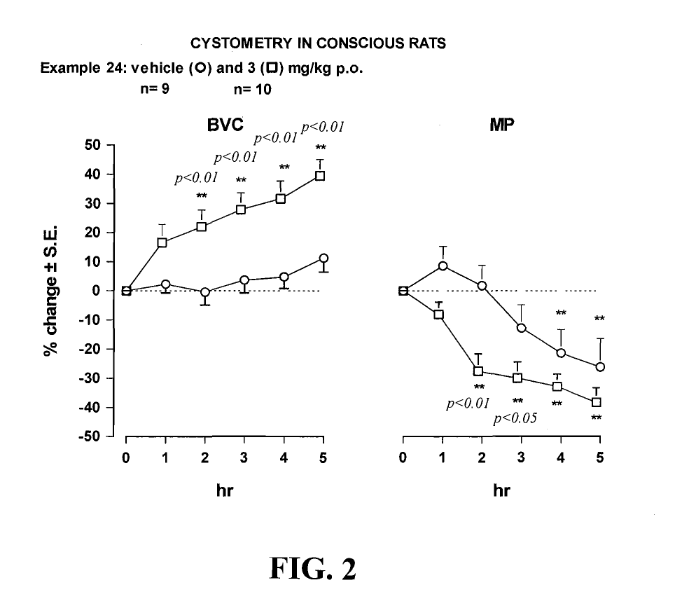

The time course of the effects of compounds of Example 16 and 24 is shown in FIGS. 1 and 2. The compound of Example 16 administered at 3 mg/kg p.o. proved effective in increasing the bladder volume capacity without significant effects on micturition pressure (FIG. 1). Also the compound of Example 24 administered at 3 mg/kg p.o. significantly increased BVC with a slight but significant reduction of the MP values observed only 2 and 3 h after administration (FIG. 2).

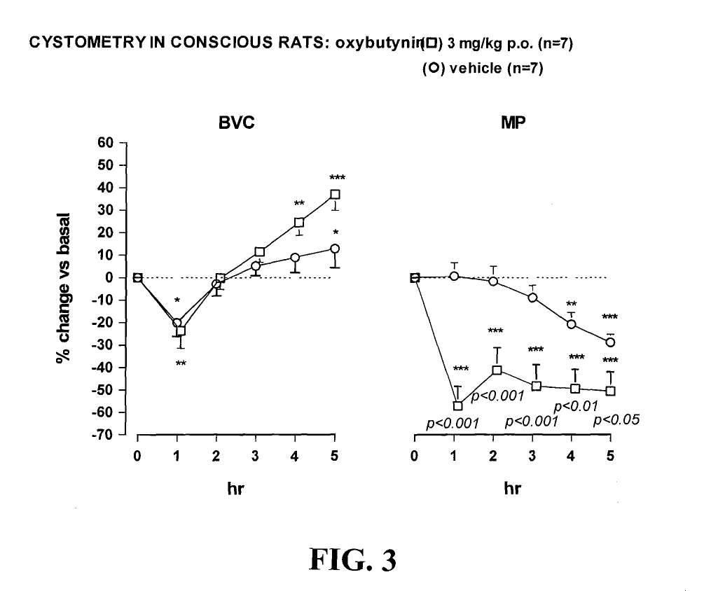

On the contrary, the oral administration of 3 mg/kg of oxybutynin induced a slight, not-significant increase of BVC in comparison with the control group. Moreover this dose greatly reduced the micturition pressure values and the differences from control animals were at all times statistically significant (FIG. 3).

### EXAMPLE 58

Behavioural Models for Studying Cognitive Enhancing Properties

The principal behavioural models used to study cognitive disorders are radial maze and fixed ratio tests:

Radial Maze: in this test male Wistar rats (300-350 body weight) are used. The animals are housed individually and maintained at 85% of their free-feeding body weight by food presented during the session and by postsession feeding. Water is freely available in the animal's home cage.

For the experiment, the following groups are used: a group of controls, a group of animals that receives vehicle and agonist, another group that receives antagonist and vehicle and a group that is treated with antagonist and agonist.

The radial maze consists of eight arms, that extended radially from a central platform that served as a starting base. At the end of each arm are positioned 15 mg food pellets as reinforcers.

During each daily session, working memory is scored on the basis of the total number of errors (which corresponded to a re-entry into the arm just visited) and the time necessary to complete the session. At the same time the pattern of arm entry is examined as angles chosen when the rat enters two consecutive arms. Since the first arm entry originates from the center of the maze, seven arm entries were measured. The frequency of each choice is then calculated as number of observations/7×100.

After 3 days of free exploration the animals are trained to complete the maze. Training continues, at the rate of one trial for day, until the rats reach the criterion of entering seven different arms in their first eight choices on 5 successive days, for a maximum of 30 days. Once the animals are successfully trained, the day of experiment serotonergic antagonist or its vehicle were administered as a 20 min pre-treatment before agonist and agonist 10 min before the start of the session.

The total number of errors and the time necessary to complete the session are evaluated and expressed as mean (±S.E.M.).

Fixed Ratio: male Sprague Dawley rats (300-350 body weight) are used. The animals are housed individually and maintained at 85% of their free-feeding body weights by food are presented during the session and by postsession feeding. Water is freely available in the animal's home cage.

For the experiment, the following groups are used: a group of controls, a group of animals that receives vehicle and agonist, another group that receives antagonist and vehicle and a group that is treated with antagonist and agonist.

Experimental sessions are conducted in a standard operant conditioning chamber. A response lever and a food trough are on the front panel of the chamber. Pellets (45 mg weight) were delivered by food trough to serve as reinforcers.

Rats are trained to respond on the right lever under an FR 30 schedule (30 response fixed ratio unit) of food presentation.

Experimental sessions consists of three 10 min components and each component is preceded by a 10 min time out period, during which time the drugs are administered.

Serotonergic antagonist or its vehicle are administered as a 30 min pretreatment before a cumulative agonist dose-effect curve. Agonist is administered cumulatively, each dose at the start of each time out period.

Data are expressed as frequency (total number of responses/total time) for each component of the session and ED₅₀ of agonist (dose that produce a 50% reduction in the response rate in presence or absence of antagonist) is then calculated.

All patents, patent applications and literature references cited in the description are hereby incorporated herein by reference in their entirety. In the case of inconsistencies, the present disclosure, including definitions, will prevail.

The present invention is not limited in scope by the specific embodiments described herein. Indeed, various modifications of the invention in addition to those described herein will become apparent to those skilled in the art from the foregoing description. Such modifications are intended to fall within the scope of the appended claims. It is further to be understood that all values given in the examples herein are approximate, and are provided for purposes of illustration.

What is claimed is:

1. A compound of formula I: 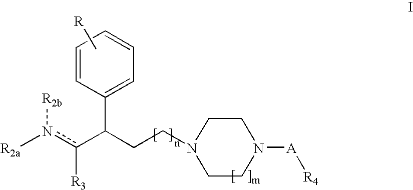
wherein
R is hydrogen or one or more substituents independently selected from the group consisting of alkyl, alkoxy, alkylthio, hydroxy, halo, polyhaloalkoxy, nitro, amino, cyano, aminocarbonyl, and alkylcarbonyl;
R₂ₐ is selected from the group consisting of hydrogen, alkyl, alkenyl, alkynyl, cycloalkyl, heteroarylalkyl, arylalkyl, hydroxy, alkoxy, alkanoyloxy, arylalkoxy, heteroarylalkoxy, alkoxycarbonyl, alkoxythiocarbonyl arylthiocarbonyl, heteroarylthiocarbonyl, alkanoyl, formyl, alkylthiocarbonyl, arylcarbonyl, alkoxyalkyl, aminocarbonyl, carbamoyl, aminothiocarbonyl, N-alkylaminocarbonyl, N-alkylaminothiocarbonyl, N,N-dialkylaminocarbonyl, N,N-dialkylaminothiocarbonyl, and heterocyclylcarbonyl or R₂ₐ is heteroarylalkyl or arylalkyl optionally substituted with one or more substituents R as given above;
R_{2b} is not present, or is selected from the group consisting of hydrogen, alkyl, alkenyl, alkynyl, cycloalkyl, heteroarylalkyl, arylalkyl, alkanoyl, formyl, alkylthiocarbonyl, arylcarbonyl, or R_{2b} is heteroarylalkyl or arylalkyl optionally substituted with one or more substituents R as given above;
R₃ is alkyl, alkenyl, alkynyl or cycloalkyl;
or
R₂ₐ and R₃ together with the nitrogen to which R₂ is attached and the carbon to which R₃ is attached, are linked to form a five membered saturated or unsaturated heterocyclic ring optionally substituted with one or more alkyl groups;
R₄ is aryl or heterocyclyl, each being optionally substituted with one or more substituent R as defined above;
A is a bond or (CH₂)ₙ;
m and n=1 or 2; [symbol]
is a single bond when R_{2b} is present or a double bond when R_{2b} is not present;
or
an enantiomer, optical isomer, diastereomer, N-oxide (e.g., N-piperazine oxide), crystalline form, hydrate, solvate or pharmaceutically acceptable salt thereof.

2. The compound of claim 1, wherein
R is halogen, polyhaloalkoxy, cyano, alkylcarbonyl, or N,N-dialkylaminocarbonyl;
R₂ₐ is selected from hydrogen, alkyl, dialkyl, aralkyl hydroxy, alkoxy, alkanoyl, heterocyclylcarbonyl, alkanoyloxy, heteroarylalkyl, heteroarylalkoxy, alkoxycarbonyl, aminocarbonyl, aminothiocarbonyl, N-alkylaminocarbonyl, N-alkylaminothiocarbonyl, N,N-dialkylaminocarbonyl, acyl, formyl thioacyl and N,N-dialkylaminothiocarbonyl;
R₃ is substituted or unsubstituted alkyl, alkenyl, alkynyl, or cycloalkyl groups; or
R₂ₐ and R₃ are linked to form a five membered lactam or isoxazole, each optionally substituted with one or more alkyl R₄ is monocyclic aryl or bicyclic heterocyclic, each optionally substituted with polyhaloalkoxy, alkoxy or halogen; and
R₄ is a bond or methylene unit.

3. The compound of claim 2, wherein
R is a fluoro;
R₂ₐ is selected from hydrogen, alkyl, and alkanoyl, aminocarbonyl, aminothiocarbonyl, N-alkylaminocarbonyl, N-alkylaminothiocarbonyl, N,N-dialkylaminocarbonyl, and N,N-dialkylaminothiocarbonyl; [symbol]
is a single bond;
R₃ is unsubstituted cycloalkyl, alkyl, or alkenyl; and
R₄ is phenyl substituted with polyhaloalkoxy, alkoxy or halogen, or is benzodioxanyl or benzimidazolyl or 2-bromo-5-methoxybenzyl or indolyl.

4. The compound of claim 3, wherein
R is 2-fluoro
R₃ is unsubstituted cyclohexyl; and
R₄ is phenyl substituted with halogen or alkoxy, or benzodioxanyl or benzimidazolyl or 2-bromo-5-methoxybenzyl or indolyl.

5. A pharmaceutical composition comprising a compound of claim 1 and a pharmaceutically acceptable excipient.

6. The pharmaceutical composition of claim 5, further comprising an antimuscarinic.

7. The pharmaceutical composition of claim 6, wherein the antimuscarinic is oxybutynin, tolterodine, darifenacin, imidafenacin, fesoterodine, propiverine, trospium or temiverine

8. The pharmaceutical composition of claim 5, further comprising an α1-adrenergic antagonist.

9. The pharmaceutical composition of claim 8, wherein the α₁-adrenergic antagonists is prazosin, doxazosin, terazosin, alfuzosin, tamsulosin and silodosin.

10. A method of reducing the frequency of bladder contractions in a mammal in need thereof comprising administering to said mammal an effective amount of at least one compound of claim 1.

11. A method of increasing urinary bladder capacity in a mammal in need thereof comprising administering to said mammal an effective amount of at least one compound of claim 1.

12. A method of treating disorders or conditions of the urinary tract in a mammal in need thereof comprising administering to said mammal an effective amount of at least one compound of claim 1.

13. The method of claim 12 wherein administering said compound ameliorates at least one condition selected from the group consisting of urinary urgency, overactive bladder, increased urinary frequency, decreased urinary compliance, cystitis, incontinence, urine leakage, enuresis, dysuria, urinary hesitancy and difficulty in emptying the bladder.

14. The method of claim 12, wherein at least one compound of claim 1 is administered to the environment of a 5-HT_{1A} receptor in an amount effective to increase the duration of bladder quiescence with no contractions.

15. A method of treating a central nervous system (CNS) disorder due to serotonergic dysfunction in a mammal in need thereof comprising administering an effective amount of at least one compound of claim 1.

16. A method of treating cognitive disorders associated with Alzheimer's disease comprising administering to a mammal in need thereof an effective amount of at least one compound of claim 1.

17. The compound of claim 1, wherein Formula I is:
1-Cyclohexyl-2-(2-fluorophenyl)-4-[4-(2-methoxyphenyl)piperazin-1-yl]butan-1-one oxime;
1-Cyclohexyl-2-(2-fluorophenyl)-4-[4-(2-methoxyphenyl)piperazin-1-yl]butan-1-one O-methyloxime;
1-Cyclohexyl-2-(2-fluorophenyl)-4-[4-(2-methoxyphenyl)piperazin-1-yl]butan-1-one O-acetyloxime;
N-{1-Cyclohexyl-2-(2-fluorophenyl)-4-[4-(2-methoxyphenyl)piperazin-1-yl]butyl}-O-methylhydroxylamine;
(RS,SR)-N-{1-Cyclohexyl-2-(2-fluorophenyl)-4-[4-(2-methoxyphenyl)piperazin-1-yl]butyl}-O-methylhydroxylamine;
(RS,RS)-N-{1-Cyclohexyl-2-(2-fluorophenyl)-4-[4-(2-methoxyphenyl)piperazin-1-yl]butyl}-O-methylhydroxylamine;
(RS,SR)-1-Cyclohexyl-2-(2-fluorophenyl)-4-[4-(2-methoxyphenyl)piperazin-1-yl]butylamine;
(RS,RS)-1-Cyclohexyl-2-(2-fluorophenyl)-4-[4-(2-methoxyphenyl)piperazin-1-yl]butylamine;
(1R,2S) 1-Cyclohexyl-2-(2-fluorophenyl)-4-[4-(2-methoxyphenyl)piperazin-1-yl]butylamine;
(1S,2R)-1-Cyclohexyl-2-(2-fluorophenyl)-4-[4-(2-methoxyphenyl)piperazin-1-yl]butylamine;
N-{1-Cyclohexyl-2-(2-fluorophenyl)-4-[4-(2-methoxyphenyl)piperazin-1-yl]butyl}formamide;
N-{(RS,SR)-1-Cyclohexyl-2-(2-fluorophenyl)-4-[4-(2-methoxyphenyl)piperazin-1-yl]butyl}acetamide;
N-Acetyl-N-{(RS,SR)-1-cyclohexyl-2-(2-fluorophenyl)-4-[4-(2-methoxyphenyl)piperazin-1-yl]butyl}acetamide;
N-{(RS,RS)-1-Cyclohexyl-2-(2-fluorophenyl)-4-[4-(2-methoxyphenyl)piperazin-1-yl]butyl}acetamide;
(RS,SR)-{1-Cyclohexyl-2-(2-fluorophenyl)-4-[4-(2-methoxyphenyl)piperazin-1-yl]butyl}methylamine;
(RS,RS)-{1-Cyclohexyl-2-(2-fluorophenyl)-4-[4-(2-methoxyphenyl)piperazin-1-yl]butyl}methylamine;
{(RS,SR)-1-Cyclohexyl-2-(2-fluorophenyl)-4-[4-(2-methoxyphenyl)piperazin-1-yl]butyl}dimethylamine;
(RS,SR)-{1-Cyclohexyl-2-(2-fluorophenyl)-4-[4-(2-methoxyphenyl)piperazin-1-yl]butyl}isopropylamine;
(RS,SR)-N-Benzyl-{1-cyclohexyl-2-(2-fluorophenyl)-4-[4-(2-methoxyphenyl)piperazin-1-yl]butyl}amine;
(RS,RS)-N-Benzyl-{1-cyclohexyl-2-(2-fluorophenyl)-4-[4-(2-methoxyphenyl)piperazin-1-yl]butyl}amine;
{(RS,SR)-1-Cyclohexyl-2-(2-fluorophenyl)-4-[4-(2-methoxyphenyl)piperazin-1-yl]butyl}furan-2-ylmethylamine;
(RS,SR)-1-Cyclohexyl-2-(2-fluorophenyl)-4-[4-(2-methoxyphenyl)piperazin-1-yl]butyl}-bis-furan-2-ylmethylamine;
(RS,SR)-N-{-1-Cyclohexyl-2-(2-fluorophenyl)-4-[4-(2-methoxyphenyl)piperazin-1-yl]butyl}-N-methylacetamide;
(RS,RS)-N-{-1-Cyclohexyl-2-(2-fluorophenyl)-4-[4-(2-methoxyphenyl)piperazin-1-yl]butyl}-N-methylacetamide;
(RS,SR)-{1-Cyclohexyl-2-(2-fluorophenyl)-4-[4-(2-methoxyphenyl)piperazin-1-yl]butyl}carbamic acid methyl ester;
{(RS,SR)-1-Cyclohexyl-2-(2-fluorophenyl)-4-[4-(2-methoxyphenyl)piperazin-1-yl]butyl}urea;
1-{(RS,SR)-1-Cyclohexyl-2-(2-fluorophenyl)-4-[4-(2-methoxyphenyl)piperazin-1-yl]butyl}-3-methylurea;
{(RS,SR)-N-1-cyclohexyl-2-(2-fluorophenyl)-4-[4-(2-methoxyphenyl)piperazin-1-yl]butyl}morpholine-4-carboxamide;
3-{(RS,SR)-1-Cyclohexyl-2-(2-fluorophenyl)-4-[4-(2-methoxy-phenyl)piperazin-1-yl]butyl}-1,1-dimethylurea;
1-{(RS,SR)-1-Cyclohexyl-2-(2-fluorophenyl)-4-[4-(2-methoxyphenyl)piperazin-1-yl]butyl}-1,3,3-trimethylurea;
1-{(RS,SR)-1-Cyclohexyl-2-(2-fluorophenyl)-4-[4-(2-methoxyphenyl)piperazin-1-yl]butyl}-3-methylthiourea;
3-{(RS,SR)-1-Cyclohexyl-2-(2-fluorophenyl)-4-[4-(2-methoxyphenyl)piperazin-1-yl]butyl}-1,1-dimethylthiourea;
3-{(1R,2S) or (1S,2R)-1-Cyclohexyl-2-(2-fluorophenyl)-4-[4-(2-methoxyphenyl)piperazin-1-yl]butyl}-1,1-dimethylthiourea;
3-{(1S,2R) or (1R,2S)-1-Cyclohexyl-2-(2-fluorophenyl)-4-[4-(2-methoxyphenyl)piperazin-1-yl]butyl}-1,1-dimethylthiourea;
2-{1-Cyclohexyl-[(E)-hydroxyiminomethyl]-3-[4-(2,3-dihydrobenzo[1,4]dioxin-5-yl)piperazin-1-yl]propyl}benzonitrile;
(R,S)-2-{1-{Cyclohexyl-[(E)-methoxyimino]methyl}-3-[4-(2,3-dihydrobenzo[1,4]dioxin-5-yl)-piperazin-1-yl]propyl}benzonitrile;
E(Z)-5-[4-(2,3-Dihydrobenzo[1,4]dioxin-5-yl)piperazin-1-yl]-3-(2-fluorophenyl)pentan-2-one oxime;
5-[4-(2,3-Dihydrobenzo[1,4]dioxin-5-yl)piperazin-1-yl]-3-(2-fluorophenyl)pentan-2-one O-methyloxime;
(RS,SR)-[4-(2,3-Dihydrobenzo[1,4]dioxin-5-yl)piperazin-1-yl]-2-(2-fluorophenyl)-1-methylbutylamine;
(RS,RS)-[4-(2,3-Dihydrobenzo[1,4]dioxin-5-yl)piperazin-1-yl]-2-(2-fluorophenyl)-1-methylbutylamine;
(RS,SR)-[4-[4-(2,3-Dihydrobenzo[1,4]dioxin-5-yl)piperazin-1-yl]-2-(2-fluorophenyl)-1-methylbutyl]methylamine;
(RS,RS)-[4-[4-(2,3-Dihydrobenzo[1,4]dioxin-5-yl)piperazin-1-yl]-2-(2-fluorophenyl)-1-methylbutyl]methylamine;
(E)-3-(2-Fluorophenyl)-5-{4-[2-(2,2,2-trifluoroethoxy)phenyl]piperazin-1-yl}pentan-2-one oxime;
(Z)-3-(2-Fluorophenyl)-5-{4-[2-(2,2,2-trifluoroethoxy)phenyl]piperazin-1-yl}pentan-2-one oxime;
(E)-3-(2-Fluorophenyl)-5-{4-[2-(2,2,2-trifluoroethoxy)phenyl]piperazin-1-yl}pentan-2-one O-methyloxime;
(Z)-3-(2-Fluorophenyl)-5-{4-[2-(2,2,2-trifluoroethoxy)phenyl]piperazin-1-yl}pentan-2-one O-methyloxime;
(RS,SR)-2-(2-Fluorophenyl)-1-methyl-4-{4-[2-(2,2,2-trifluoroethoxy)phenyl]piperazin-1-yl}butylamine;
(RS,RS)-2-(2-Fluorophenyl)-1-methyl-4-{4-[2-(2,2,2-trifluoroethoxy)phenyl]piperazin-1-yl}butylamine;
5-{3-[4-(2,3-Dihydrobenzo[1,4]dioxin-5-yl)piperazin-1-yl]-1-phenylpropyl}pyrrolidin-2-one;
(RS,SR)-5-{3-[4-(2,3-Dihydrobenzo[1,4]dioxin-5-yl)piperazin-1-yl]-1-phenylpropyl}-1-ethylpyrrolidin-2-one;
(RS,RS)-5-{3-[4-(2,3-Dihydrobenzo[1,4]dioxin-5-yl)piperazin-1-yl]-1-phenylpropyl}-1-ethylpyrrolidin-2-one;
5-{3-[4-(2,3-Dihydrobenzo[1,4]dioxin-5-yl)piperazin-1-yl]-1-phenylpropyl}-1-ethylpyrrolidin-2-one;
1-(2,3-Dihydrobenzo[1,4]dioxin-5-yl)-4-[3-(5-methylisoxazol-3-yl)-3-phenylpropyl]piperazine;
N-[1-Cyclohexyl-4-[4-(2,3-dihydrobenzo[1,4]dioxin-5-yl)piperazin-1-yl]-2-(2-fluorophenyl)butyl]formamide;
(RS,SR)-1-Cyclohexyl-4-[4-(2,3-dihydrobenzo[1,4]dioxin-5-yl)piperazin-1-yl]-2-(2-fluorophenyl)butylamine;
1-Cyclohexyl-4-[4-(2,3-dihydrobenzo[1,4]dioxin-5-yl)piperazin-1-yl]-2-(2-fluoro-phenyl)butylmethylamine;
N-[1-Cyclohexyl-4-[4-(2,3-dihydrobenzo[1,4]dioxin-5-yl)piperazin-1-yl]-2-(2-fluoro-phenyl)butyl]acetamide;
N-[1-Cyclohexyl-4-[4-(2,3-dihydrobenzo[1,4]dioxin-5-yl)piperazin-1-yl]-2-(2-fluoro-phenyl)butyl]-N-methylacetamide;
N-[4-[4-(2-Bromo-5-methoxybenzyl)piperazin-1-yl]-1-cyclohexyl-2-(2-fluorophenyl)butyl]formamide;
4-[4-(2-Bromo-5-methoxybenzyl)piperazin-1-yl]-1-cyclohexyl-2-(2-fluorophenyl)butylamine;
4-[4-(2-Bromo-5-methoxybenzyl)piperazin-1-yl]-1-cyclohexyl-2-(2-fluorophenyl)butylmethylamine;
N-[4-[4-(2-Bromo-5-methoxybenzyl)piperazin-1-yl]-1-cyclohexyl-2-(2-fluoro-phenyl)butyl]acetamide;
N-[4-[4-(2-Bromo-5-methoxybenzyl)piperazin-1-yl]-1-cyclohexyl-2-(2-fluorophenyl)butyl]-N-methylacetamide;
N-[4-[4-(1H-Benzimidazol-4-yl)piperazin-1-yl]-1-cyclohexyl-2-(2-fluoro-phenyl)butyl]formamide;
4-[4-(1H-Benzimidazol-4-yl)piperazin-1-yl]-1-cyclohexyl-2-(2-fluorophenyl)butylamine;
(RS,SR)-4-[4-(1H-Benzimidazol-4-yl)piperazin-1-yl]-1-cyclohexyl-2-(2-fluorophenyl)butylamine;
(RS,RS)-4-[4-(1H-Benzimidazol-4-yl)piperazin-1-yl]-1-cyclohexyl-2-(2-fluorophenyl)butylamine;
4-[4-(1H-Benzimidazol-4-yl)piperazin-1-yl]-1-cyclohexyl-2-(2-fluorophenyl)butylmethylamine;
N-[4-[4-(1H-Benzimidazol-4-yl)piperazin-1-yl]-1-cyclohexyl-2-(2-fluorophenyl)butyl]acetamide;
N-[4-[4-(1H-Benzimidazol-4-yl)piperazin-1-yl]-1-cyclohexyl-2-(2-fluorophenyl)butyl]-N-methylacetamide;
N-{1-Cyclohexyl-2-(2-fluorophenyl)-4-[4-(1H-indol-4-yl)piperazin-1-yl]butyl}formamide;
1-{(RS,SR)-1-Cyclohexyl-2-(2-fluorophenyl)-4-[4-(2-methoxyphenyl)piperazin-1-yl]butyl}-1,3,3-trimethylthiourea; or an enantiomer, optical isomer, diastereomer, N-oxide (e.g., N-piperazine oxide), crystalline form, hydrate, solvate or pharmaceutically acceptable salt thereof.
